(19) United States
(12) Patent Application Publication
(10) Pub. No.: US 2017/0325525 A1
(43) Pub. Date: Nov. 16, 2017

(54) SYSTEMS, DEVICES, AND METHODS INCLUDING A LIFT GARMENT

(71) Applicant: Elwha LLC, Bellevue, WA (US)

(72) Inventors: Roderick A. Hyde, Redmond, WA (US); Jordin T. Kare, San Jose, CA (US); Eric C. Leuthardt, St. Louis, MO (US); Richard T. Lord, Gig Harbor, WA (US); Robert W. Lord, Seattle, WA (US); Clarence T. Tegreene, Mercer Island, WA (US); Lowell L. Wood, Jr., Bellevue, WA (US); Victoria Y.H. Wood, Livermore, CA (US)

(73) Assignee: Elwha LLC, Bellevue, WA (US)

(21) Appl. No.: 15/153,255

(22) Filed: May 12, 2016

(51) Int. Cl. A41D 13/12; A61B 5/00; A61B 5/0205; A41D 1/00; A61G 7/10; A61B 5/024; A61B 5/01; A61B 5/021; A61B 5/08; A61B 5/1477

(52) U.S. Cl. CPC A41D 13/1263; A41D 1/002; A61B 5/6804; A61B 5/02055; A61G 7/1051; A41D 2400/48; A61B 5/01; A61B 5/021; A61B 5/08; A61B 5/1477; A61B 5/02438

(57) ABSTRACT

Devices, systems, and methods are described including a lift garment having a fabric-like material shaped to encircle a torso and at least a portion arms and legs of a subject; at least one lift attachment element associated with the fabric-like material at at least one of one or more lift attachment sites, the at least one lift attachment element configured to attach the lift garment to a lift apparatus; one or more physiological sensors configured to measure at least one physiological parameter of the subject; a microcontroller including circuitry configured to receive and process information regarding the measured at least one physiological parameter of the subject; and a reporting device operably coupled to the microcontroller and configured to transmit one or more signals indicative of the processed information regarding the measured at least one physiological parameter of the subject.

If an Application Data Sheet (ADS) has been filed on the filing date of this application, it is incorporated by reference herein. Any applications claimed on the ADS for priority under 35 U.S.C. §§119, 120, 121, or 365(c), and any and all parent, grandparent, great-grandparent, etc. applications of such applications, are also incorporated by reference, including any priority claims made in those applications and any material incorporated by reference, to the extent such subject matter is not inconsistent herewith.

### CROSS-REFERENCE TO RELATED APPLICATIONS

The present application claims the benefit of the earliest available effective filing date(s) from the following listed application(s) (the “Priority Applications”), if any, listed below (e.g., claims earliest available priority dates for other than provisional patent applications or claims benefits under 35 USC §119(e) for provisional patent applications, for any and all parent, grandparent, great-grandparent, etc. applications of the Priority Application(s)).

### PRIORITY APPLICATIONS

None

If the listings of applications provided above are inconsistent with the listings provided via an ADS, it is the intent of the Applicant to claim priority to each application that appears in the Domestic Benefit/National Stage Information section of the ADS and to each application that appears in the Priority Applications section of this application.

All subject matter of the Priority Applications and of any and all applications related to the Priority Applications by priority claims (directly or indirectly), including any priority claims made and subject matter incorporated by reference therein as of the filing date of the instant application, is incorporated herein by reference to the extent such subject matter is not inconsistent herewith.

### SUMMARY

In an aspect, a lift garment includes, but is not limited to, a fabric-like material shaped to substantially completely encircle a torso and at least a portion of arms and legs of a subject, the fabric-like material including one or more lift attachment sites; at least one lift attachment element associated with the fabric-like material at at least one of the one or more lift attachment sites, the at least one lift attachment element configured to attach the lift garment to a lift apparatus; a load sensor configured to measure a load, the load sensor associated with at least one of the one or more lift attachment sites or along at least one load path between the one or more lift attachment sites; a microcontroller including circuitry configured to receive and process information regarding the measured load; and a reporting device operably coupled to the microcontroller and configured to transmit one or more signals indicative of the processed information regarding the measured load. In addition to the foregoing, other aspects of a lift garment are described in the claims, drawings, and text forming a part of the present disclosure.

In an aspect, a system includes, but is not limited to, a lift garment including a fabric-like material shaped to substantially completely encircle a torso and at least a portion of arms and legs of a subject, the fabric-like material including one or more lift attachment sites; at least one lift attachment element associated with the fabric-like material at at least one of the one or more lift attachment sites, the at least one lift attachment element configured to attach the lift garment to a lift apparatus; a load sensor configured to measure a load, the load sensor associated with at least one of the one or more lift attachment sites or along at least one load path between the one or more lift attachment sites; a microcontroller including circuitry configured to receive and process information regarding the measured load; and a reporting device operably coupled to the microcontroller and configured to transmit one or more signals indicative of the processed information regarding the measured load; and a lift control mechanism including a receiver configured to receive the one or more signals from the reporting device indicative of the processed information regarding the measured load; and circuitry configured to control a function of the lift apparatus in response to the one or more signals received from the reporting device. In addition to the foregoing, other aspects of a system are described in the claims, drawings, and text forming a part of the present disclosure.

In an aspect, a method implemented with a lift garment includes, but is not limited to, measuring a load value with a load sensor associated with the lift garment worn by a subject and attached to a lift apparatus, the lift garment including a fabric-like material shaped to substantially completely encircle a torso and at least a portion of arms and legs of the subject, the load sensor; at least one lift attachment element associated with the fabric-like material at at least one of one or more lift attachment sites; a microcontroller including circuitry and a stored range of acceptable load values; and a reporting device operably coupled to the microcontroller; receiving and processing the measured load value with the circuitry of the microcontroller; determining whether the measured load value falls within the stored range of acceptable load values; and transmitting one or more signals from the reporting device indicative of whether the measured load value falls within the stored range of acceptable load values. In addition to the foregoing, other aspects of a method are described in the claims, drawings, and text forming a part of the present disclosure.

In an aspect, a lift garment includes, but is not limited to, a fabric-like material shaped to substantially completely encircle a torso and at least a portion of arms and legs of a subject, the fabric-like material including one or more lift attachment sites; at least one lift attachment element associated with the fabric-like material at at least one of the one or more lift attachment sites, the at least one lift attachment element configured to attach the lift garment to a lift apparatus; one or more physiological sensors configured to measure at least one physiological parameter of the subject; a microcontroller including circuitry configured to receive and process information regarding the measured at least one physiological parameter of the subject; and a reporting device operably coupled to the microcontroller and configured to transmit one or more signals indicative of the processed information regarding the measured at least one physiological parameter of the subject. In addition to the foregoing, other aspects of a lift garment are described in the claims, drawings, and text forming a part of the present disclosure.

In an aspect, a system includes, but is not limited to, a lift garment including a fabric-like material shaped to substantially completely encircle a torso and at least a portion of arms and legs of a subject, the fabric-like material including one or more lift attachment sites; at least one lift attachment element associated with the fabric-like material at at least one of the one or more lift attachment sites, the at least one lift attachment element configured to attach the lift garment to a lift apparatus; one or more physiological sensors configured to measure at least one physiological parameter of the subject; a microcontroller including circuitry configured to receive and process information regarding the measured at least one physiological parameter of the subject; and a reporting device operably coupled to the microcontroller and configured to transmit one or more signals indicative of the processed information regarding the measured at least one physiological parameter of the subject; and a lift control mechanism including a receiver configured to receive the one or more signals from the reporting device indicative of the processed information regarding the measured at least one physiological parameter of the subject; and circuitry configured to control a function of the lift apparatus in response to the one or more signals received from the reporting device. In addition to the foregoing, other aspects of a system are described in the claims, drawings, and text forming a part of the present disclosure.

In an aspect, a method implemented with a lift garment includes, but is not limited to, measuring at least one physiological parameter of a subject with one or more physiological sensors associated with the lift garment worn by a subject, the lift garment including a fabric-like material shaped to substantially completely encircle a torso and at least a portion of arms and legs of the subject; the one or more physiological sensors; at least one lift attachment element associated with the fabric-like material at at least one of one or more lift attachment sites; a microcontroller including circuitry and a stored range of acceptable physiological parameter values; and a reporting device operably coupled to the microcontroller; receiving and processing information regarding the measured at least one physiological parameter of the subject with the circuitry of the microcontroller; and transmitting one or more control signals from the reporting device to a lift apparatus based on the processed information regarding the measured at least one physiological parameter of the subject. In addition to the foregoing, other aspects of a method are described in the claims, drawings, and text forming a part of the present disclosure.

In an aspect, a wearable lift device includes, but is not limited to, a flexible material having a shape sufficient to substantially completely encircle at least a portion of a subject's body; at least one fastener configured to secure the flexible material around the at least a portion of the subject's body; at least one lift attachment element associated with the flexible material at one or more lift attachment sites, the at least one lift attachment element configured to attach the wearable lift device to a lift apparatus; a load sensor configured to measure a load, the load sensor associated with at least one of the one or more lift attachment sites or along a load path between the one or more lift attachment sites; a microcontroller including circuitry configured to receive and process information regarding the measured load; and a reporting device operably coupled to the microcontroller and configured to transmit one or more signals indicative of the processed information regarding the measured load. In addition to the foregoing, other aspects of a wearable lift device are described in the claims, drawings, and text forming a part of the present disclosure.

In an aspect, a system includes, but is not limited to, a wearable lift device including a flexible material having a shape sufficient to substantially completely encircle at least a portion of a subject's body; at least one fastener configured to secure the flexible material around the at least a portion of the subject's body; at least one lift attachment element associated with the flexible material at one or more lift attachment sites, the at least one lift attachment element configured to attach the wearable lift device to a lift apparatus; a load sensor configured to measure a load, the load sensor associated with at least one of the one or more lift attachment sites or along a load path between the one or more lift attachment sites; a microcontroller including circuitry configured to receive and process information regarding the measured load; and a reporting device operably coupled to the microcontroller and configured to transmit one or more signals indicative of the processed information regarding the measured load; and a lift control mechanism including a receiver configured to receive the one or more signals from the reporting device indicative of the processed information regarding the measured load; and circuitry configured to control a function of the lift apparatus in response to the one or more signals received from the reporting device of the wearable lift device. In addition to the foregoing, other aspects of a system are described in the claims, drawings, and text forming a part of the present disclosure.

In an aspect, a lift sling includes, but is not limited to, a fabric-like material having a shape sufficient to at least partially cover a portion of a subject's body; at least one lift attachment element associated with the fabric-like material at one or more lift attachment sites, the at least one lift attachment element configured to attach the lift sling to a lift apparatus; one or more physiological sensors configured to measure at least one physiological parameter of the subject; a microcontroller including circuitry configured to receive and process information regarding the measured at least one physiological parameter of the subject; and a reporting device operably coupled to the microcontroller and configured to transmit one or more signals indicative of the processed information regarding the measured at least one physiological parameter of the subject. In addition to the foregoing, aspects of a lift sling are described in the claims, drawings, and text forming a part of the present disclosure.

In an aspect, a system includes, but is not limited to, a lift sling having a shape sufficient to at least partially cover a portion of a subject's body, the lift sling including at least one lift attachment element configured to attach the lift sling to a lift apparatus; at least one blood oxygenation sensor; a microcontroller including circuity configured to receive one or more signals from the at least one blood oxygenation sensor and configured to determine a level of hypoxia of the subject; and a transmission unit operably coupled to the microcontroller and configured to transmit one or more control signals to the lift apparatus to control an operation of the lift apparatus based on the determined level of hypoxia of the subject. In addition to the foregoing, aspects of a system are described in the claims, drawings, and text forming a part of the present disclosure.

In an aspect, a method implemented with a wearable lift device includes, but is not limited to, measuring a load value with a one load sensor associated with the wearable lift device worn by a subject and attached to a lift apparatus, the wearable lift device including the load sensor; a flexible material shaped to substantially completely encircle at least a portion of the subject's body; at least one fastener configured to secure the flexible material around the at least a portion of the subject's body; at least one lift attachment element associated with the flexible material at at least one of one or more lift attachment sites; a microcontroller including circuitry and a stored range of acceptable load values; and a reporting device operably coupled to the microcontroller; receiving and processing the measured load value with the circuitry of the microcontroller; determining whether the measured load value falls within the stored range of acceptable load values; and transmitting one or more control signals from the reporting device to the lift apparatus to control an operation of the lift apparatus based on whether the measured load value falls within the stored range of acceptable load values. In addition to the foregoing, aspects of a method are described in the claims, drawings, and text forming a part of the present disclosure.

In an aspect, a method implemented with a wearable lift device includes, but is not limited to, measuring at least one physiological parameter of a subject with one or more physiological sensors associated with the wearable lift device worn by a subject and attached to a lift apparatus, the wearable lift device including the one or more physiological sensors; a flexible material shaped to substantially completely encircle at least a portion of the subject's body; at least one fastener configured to secure the flexible material around the at least a portion of the subject's body; at least one lift attachment element associated with the flexible material at at least one of one or more lift attachment sites; a microcontroller including circuitry and a stored range of acceptable physiological parameter values; and a reporting device operably coupled to the microcontroller; receiving and processing the measured at least one physiological parameter of the subject with the circuitry of the microcontroller; determining whether the measured at least one physiological parameter of the subject falls within the stored range of acceptable physiological parameter values; and transmitting one or more control signals from the reporting device to the lift apparatus to control an operation of the lift apparatus based on whether the measured at least one physiological parameter of the subject falls within the stored range of acceptable physiological parameter values. In addition to the foregoing, aspects of a method are described in the claims, drawings, and text forming a part of the present disclosure.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

FIG. 1 is a block diagram of a lift garment including a load sensor.

FIG. 2A shows an embodiment of a lift garment.

FIG. 2B shows an embodiment of a lift garment attached to a lift apparatus.

FIG. 3A shows an embodiment of a lift garment including a first reinforcing material.

FIG. 3B shows an embodiment of a lift garment including a second reinforcing material.

FIG. 3C shows an embodiment of a lift garment including a structural.

FIG. 4 is a block diagram illustrating further aspects of a lift garment including a load sensor.

FIG. 5 is a block diagram illustrating further aspects of a lift garment including a load sensor.

FIG. 6 is a block diagram illustrating further aspects of a lift garment including a load sensor.

FIG. 7 illustrates aspects of a system including a lift garment and a lift control mechanism.

FIG. 8 shows further aspects of a system including a lift garment and a lift control mechanism.

FIG. 9 shows further aspects of a system including a lift garment and a lift control mechanism.

FIG. 10 shows a flow diagram of a method implemented with a lift garment including a load sensor.

FIG. 11 is a block diagram of a lift garment including one or more physiological sensors.

FIG. 12 is a block diagram illustrating further aspects of a lift garment including one or more physiological sensors.

FIG. 13 is a block diagram illustrating further aspects of a lift garment including one or more physiological sensors.

FIG. 14 illustrates aspects of a system including a lift garment and a lift control mechanism.

FIG. 15 shows a flow diagram of a method implemented with a lift garment including one or more physiological sensors.

FIG. 16 is a block diagram of a wearable lift device including a load sensor.

FIG. 17A shows an embodiment of a wearable lift device.

FIG. 17B shows an embodiment of a wearable lift device attached to a lift apparatus.

FIG. 18 is a block diagram illustrating further aspects of a wearable lift device including a load sensor.

FIG. 19 is a block diagram illustrating further aspects of a wearable lift device including a load sensor.

FIG. 20 is a block diagram illustrating further aspects of a wearable lift device including a load sensor.

FIG. 21 illustrates aspects of a system including a wearable lift device and a lift control mechanism.

FIG. 22A shows an embodiment of a lift sling including one or more physiological sensors.

FIG. 22B shows an embodiment of a lift sling including one or more physiological sensors attached to a lift apparatus.

FIG. 23 is a block diagram illustrating further aspects of a lift sling including one or more physiological sensors.

FIG. 24 shows a flow diagram of a method implemented with a wearable lift device including a load sensor.

FIG. 25 shows a flow diagram of a method implemented with a wearable lift device including one or more physiological sensors.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

Described herein are devices, systems, and methods of use in lifting a subject. In an aspect, a lift garment is described for use with a lift apparatus (e.g., a Hoyer-like lift, a patient lift, a jack hoist, or a hydraulic lift). In some embodiments, the lift garment is designed for continuous or long-term wear by a subject to allow for convenient transfer of the subject with a lift apparatus from one position or place to another position or place. In some embodiments, the lift garment is configured for use with a lift apparatus to aid in transferring a subject from one bed to another bed. In some embodiments, the lift garment is configured for use with a lift apparatus to aid in transferring a subject from a bed to a chair. In some embodiments, the lift garment is configured for use with a lift apparatus to aid in helping a subject reach a standing position. In an aspect, the lift garment is configured for use in a hospital, skilled nursing, or assisted living facility. For example, the lift garment can be configured for use in transferring a patient in a hospital or skilled nursing facility from a bed to a wheelchair. In an aspect, the lift garment is configured for use in a residential setting. For example, the lift garment can be configured for use in lifting a subject who has fallen on the floor in an assisted or independent living facility. For example, the lift garment can be configured for transferring a limited mobility subject from one position to another in a residential setting. In some embodiments, the lift garment is configured such that the subject is able to perform the transfer procedure unaided, i.e., in the absence of a caregiver, allowing for increased independence. The lift apparatus can include a floor or mobile lift apparatus, a ceiling-mounted lift apparatus, a stand-assist lift apparatus, a sit-to-stand lift, and/or a wall-mounted lift apparatus.

In some embodiments, a lift garment is designed for suspending/supporting a subject outside of a clinical or medical setting. For example, a lift garment, such as described herein, is contemplated for use mountain/rock climbing and/or caving; helicopter and/or fire rescue; safety gear used for certain occupations that involve working at elevation—window washers, house painters, utility pole repair persons, roofers, construction worker, and the like.

In some embodiments, a lift garment includes a load sensor configured to measure a load. For example, the lift garment can include a load sensor configured to measure the load of a subject as he or she is wearing the lift garment and lifted by a lift apparatus. For example, one or more load sensors associated with the lift garment can be used to determine whether the load of the subject is distributed appropriately, e.g., evenly, within the lift garment.

In some embodiments, a lift garment includes one or more physiological sensors configured to measure at least one physiological parameter of a subject. For example, the lift garment can include one or more physiological sensors configured to measure at least one physiological parameter of the subject predictive of hypoxia, e.g., heart rate, blood oxygenation, blood pressure, and/or respiration rate. For example, the lift garment can include one or more physiological sensors configured to measure a physiological symptom of suspension trauma, e.g., altered heart and/or respiration rate, changes in blood pressure and/or oxygenation, and the like. See, e.g., Lee & Porter (2007) “Suspension trauma,” Emerg. Med. J. 24:237-238, which is incorporated herein by reference.

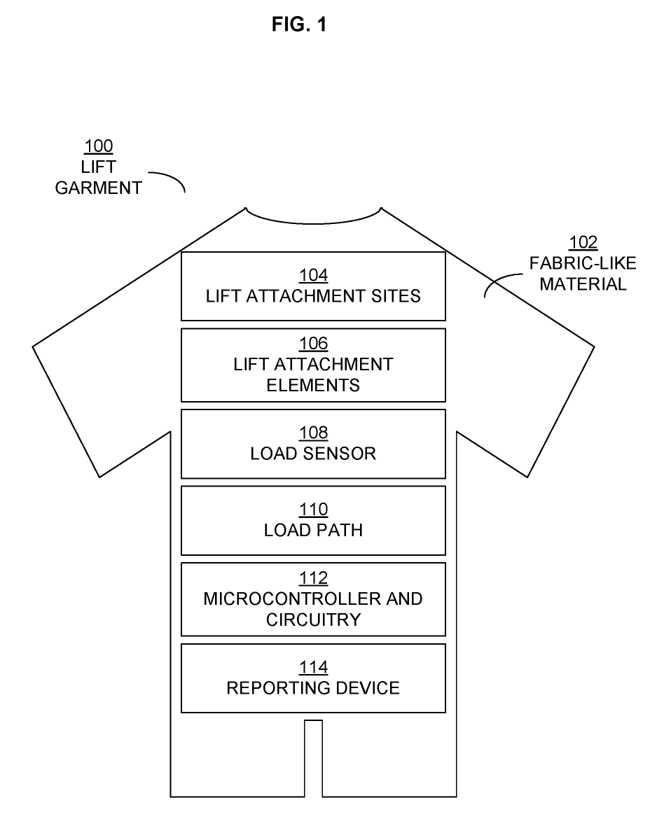

With reference to FIG. 1, shown is an example of a lift garment 100 which can serve as a context for introducing one or more devices, systems, and/or processes described herein. FIG. 1 shows aspects of a lift garment 100. Lift garment 100 includes a fabric-like material 102 shaped to substantially completely encircle a torso and at least a portion of arms and legs of a subject. Fabric-like material 102 further includes one or more lift attachment sites 104. Lift garment 100 further includes at least one lift attachment element 106 associated with the fabric-like material 102 at at least one of the one or more lift attachment sites 104. The at least one lift attachment element 106 is configured to attach the lift garment 100 to a lift apparatus. Lift garment 100 further includes a load sensor 108 configured to measure a load. Load sensor 108 is associated with at least one of the one or more lift attachment sites 104 or along at least one load path 110 between the one or more lift attachment sites 104. Lift garment 100 further includes a microcontroller 112 including circuitry configured to receive and process information regarding the measured load. Lift garment 100 further includes a reporting device 114 operably coupled to microcontroller 112 and configured to transmit one or more signals indicative of the processed information regarding the measured load value. For example, the reporting device can transmit one or more of an optical, audio, or haptic signal indicative of the processed information regarding the measured load value. For example, the reporting device can transmit a wireless signal indicative of the processed information regarding the measured load.

In some embodiments, microcontroller 112 includes a stored range of acceptable load values and circuitry configured to determine if the measured load falls within the range of acceptable load values. In some embodiments, microcontroller 112 includes circuitry configured to receive and process the information regarding the measured load, determine whether the measured load falls within a range of acceptable load values, and transmit one or more signals indicative of the processed information. For example, the reporting device can emit an optical signal, e.g., a red or green light, indicating whether the measured load falls within a range of acceptable load values.

In some embodiments, microcontroller 112 includes a stored range of acceptable load values and circuitry configured to determine if the measured load falls within the range of acceptable load values, and reporting device 114 is configured to transmit a locking signal to the lift apparatus if the measured load fails to fall within the range of acceptable load values. For example, the reporting device can transmit a locking signal to the lift apparatus to block an operation of the lift apparatus if the measured load is too high (e.g., due to excess weight of the subject or uneven distribution of the subject's weight) for the lift garment and/or lift apparatus. In an aspect, reporting device 114 is configured to transmit a control signal in response to the processed information regarding the measured load. For example, the reporting device can transmit a control signal to the lift apparatus to at least one of turn on or off the lift apparatus, activate a lifting or a lowering function, and/or control the speed or acceleration of the lifting or lowering function.

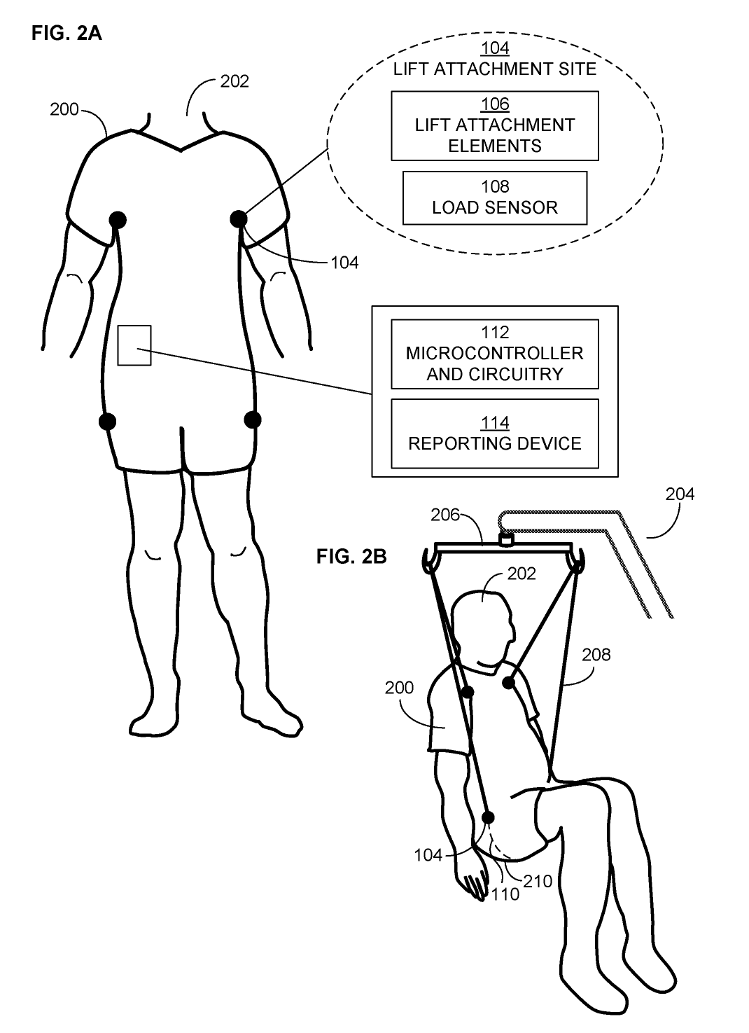

FIGS. 2A and 2B illustrate further aspects of a lift garment. FIG. 2A shows an embodiment of a lift garment 200 being worn by subject 202. In this non-limiting example, the fabric-like material of the lift garment 200 is shaped with short sleeves and short legs. For example, the fabric-like material of the lift garment can be shaped as a onesie or uni-suit with short and/or long sleeves and pant legs. Lift garment 200 includes lift attachment sites 104. In this non-limiting example, lift garment 200 includes four lift attachment sites 104. However, as few as one lift attachment site or as many as twenty lift attachment sites are contemplated, depending upon the configuration of the lift garment and/or the attachment site on the lift apparatus. Each of the lift attachment sites 104 include at least one lift attachment element 106 (e.g., a hook, a loop of material, or a magnet), and a load sensor 108 configured to measure a load associated with the lift attachment site 104. Lift garment 200 further includes a microcontroller 112 including circuitry and reporting device 114. In this non-limiting example, a central microcontroller 112 and reporting device 114 receive, process, and transmit information regarding the measured load at each of the lift attachment sites 104 or along a load path between one or more lift attachment sites 104. In some embodiments, each of the lift attachment sites includes a microcontroller and a reporting device to receive, process, and transmit information regarding the measured load at a specific lift attachment site.

FIG. 2B shows lift garment 200 being worn by subject 202 and attached to lift apparatus 204. In an aspect, lift apparatus 204 includes a Hoyer-like lift apparatus. Lift garment 200 is attached to an attachment portion 206 of lift apparatus 204 through straps 208 connected to the lift attachment elements, e.g., hook or loops of material, associated with lift attachment sites 104. Also shown in FIG. 2B is a non-limiting example of a load path 110 (dotted line) extending along the buttocks 210 of subject 202 between lift attachment sites 104 on either side of lift garment 200. In some embodiments, one or more load sensors are associated with at least one load path between one or more lift attachment sites. In this non-limiting example, one or more load sensors can be associated with load path 210 to measure the load distributed between the lift attachment sites 104 (in this example, under the buttocks of the subject).

### Fabric-Like Material

In some embodiments, a lift garment includes a fabric-like material shaped to substantially completely encircle a torso and at least a portion of arms and legs of a subject. For example, the fabric-like material can be shaped as a one piece, “union suit” with short sleeves and pant legs. In an aspect, a lift garment includes a fabric-like material shaped to substantially completely encircle the torso and the at least a portion of the arms and legs of a human subject. In an aspect, a lift garment includes a fabric-like material shaped in various sizes. For example, the fabric-like material can be shaped in at least small, medium, large, extra-large, and plus size configurations to accommodate subjects of varied size and weight. In an aspect, a lift garment includes a fabric-like material shaped to fit an infant or small child. In an aspect, a lift garment includes a fabric-like material shaped to fit an obese subject (e.g., body-mass index greater than 30).

In an aspect, a lift garment includes a fabric-like material shaped to substantially completely encircle the torso and the at least a portion of the arms and legs of a non-human subject. For example, the fabric-like material can be shaped for use with a large animal, e.g., a domesticated or non-domesticated large animal. For example, the fabric-like material can be shaped for use with a horse, e.g., a thoroughbred racing horse. For example, the fabric-like material can be shaped for use with an elephant, giraffe, rhinoceros, or other large animals in a zoo or wild-life preserve.

In an aspect, a lift garment includes at least one fastener. In an aspect, the lift garment can include at least one fastener for closing the lift garment around the subject. For example, the lift garment can include a zipper along a front portion of the lift garment that allows the subject to get into and out of the lift garment. For example, the fabric-like material of the lift garment can include a zipper, buttons, snaps, or other means for fastening parts of the fabric-like material around the torso and/or the arms and legs of the subject for ease of putting on and/or taking off the lift garment. Other non-limiting examples of fasteners include a buckle, a cinch, a hook and loop fastener, a belt, a hook and eye fastener, or a snap.

In an aspect, the fabric-like material is formed from fibers, filaments, or yarns. In an aspect, the fabric-like material generally has a two-dimensional structure (i.e., a length and width that are substantially greater than a thickness). In an aspect, the fabric-like material is formed from an animal, plant, mineral, or synthetic source. In an aspect, the fabric-like material is formed from an animal source, e.g., at least one of leather, wool, or silk. In an aspect, the fabric-like material is formed from a plant source, e.g., at least one of cotton, flax, jute, hemp, modal, regenerated cellulose, bamboo, pina, or ramie. In an aspect, the fabric-like material is formed from a mineral source, e.g., at least one of basalt fibers, glass fibers, or metal fibers. In an aspect, the fabric-like material is formed from a synthetic material, e.g., polyester, aramid fiber, acrylic, nylon, polyurethane, olefin fiber, or polylactide fiber. In an aspect, at least a portion of the fabric-like material includes a woven material or a knit material. For example, the fabric-like material can be formed from weaving a yarn or a plurality of yarns using a loom. For example, the fabric-like material can include a woven material formed from at least one of wool, silk, cotton, flax, jute, asbestos, glass, fiber, nylon, polyester, acrylic, or a combination thereof. For example, the fabric-like material can be formed from interlooping a yarn or a plurality of yarns using a knitting machine. For example, the fabric-like material can include a knit material formed from at least one of wool, silk, cotton, flax, jute, asbestos, glass, fiber, nylon, polyester, acrylic, or a combination thereof.

In an aspect, at least a portion of the fabric-like material is formed from a non-woven material. For example, the fabric-like material can include sheet or web structures bonded together by entangling fiber or filaments mechanically, thermally, or chemically. For example, the fabric-like material can include a non-woven material formed by fibers bounded together by chemical, mechanical, heat, and/or solvent treatment. For example, the fabric-like material can include a non-woven material formed by pressing fibers together under heat and/or pressure with or without an added binder. For example, the fabric-like material can include a form of TYVEK (DuPont).

In an aspect, at least a portion of the fabric-like material includes at least one polymer type. In an aspect, the at least one polymer type can be incorporated into a knit, woven, or non-woven material. The at least one polymer type can include nylon, polyester, rayon, Tyvek, polymerized chloroprene (neoprene), polyvinylchloride, polyethylene terephthalate (PET), polypropylene, etc. spun bound olefin fiber, For example, the fabric-like material can include a non-woven material formed from polyethylene terephthalate or polypropylene.

In an aspect, at least a portion of the fabric-like material includes a stretchable material configured to fit snuggly against the surface of a subject. For example, the fabric-like material can include at least in part a material including elastic fibers (e.g., Lycra) that stretches at least a portion of the lift garment tightly against a skin surface of a human subject to facilitate good contact between the skin surface and a physiological sensor associated with the lift garment.

In an aspect, the fabric-like material includes a heavy duty fabric. For example, the fabric-like material can include a type of heavy duty cotton canvas. For example, the fabric-like material can include a heavy duty cotton duck cloth. In an aspect, the fabric-like material includes a form of ballistic nylon. For example, the fabric-like material can include vinyl sheeting or vinyl coated polyester mesh. In an aspect, the fabric-like material includes a form of CORDURA fabric (from, INVISTA).

In an aspect, the fabric-like material includes one or more electronic threads that incorporates one or more conductive materials (e.g., metallic, semi-conductive) to facilitate electric transmissions throughout at least a portion of the fabric-like material. In an aspect, the conductive material (e.g., a metal wire) is twisted around a yarn (e.g., a polymer yarn). In an aspect, yarn (e.g., a polymer yarn) is physically/chemically coated with a thin layer of conductive material (e.g., a metal coating). For example, at least a portion of the fabric-like material can be woven or knit with a conductive yarn (from, e.g., Textronics®, Inc. Chadds Ford, Pa.). For example, at least a portion of the fabric-like material can include electrically conductive, flexible metal-coated fabrics including wovens, nonwovens, and knits, filaments, and yarns (from, e.g., Swift Textile Metalizing LLC, Bloomfield, Conn.). For example, fabric-like material can include electrically conductive thread or yarn woven and/or integrated within a weave pattern of the fabric-like material to form at least a portion of a sensor, the microcontroller and circuitry, and/or the reporting device. In an aspect, the conductive material (e.g., metal fibers) twisted/combined to form a conductive yarn of metal multifilaments. For example, the fabric-like material can include metal monofilaments that can be blended with fibers (e.g., cotton, polyester, polyamides, or aramides) or directly woven or knitted into the fabric-like material. Non-limiting examples of metal monofilaments include copper, silver-plated copper, brass, silver-plated brass, aluminum, or copper-clad aluminum. See, e.g., Stoppa & Chiolerio (2014) “Wearable electronics and smart textiles: A critical review,” Sensors 14:11957-11992, which is incorporated herein by reference.

### Lift Attachment Sites and Load Paths

The lift garment includes one or more lift attachment sites. In some embodiments, the lift garment includes from one to twenty lift attachment sites. For example, the lift garment can include one, two, three, four, five, six, seven, eight, nine, ten, eleven, twelve, thirteen, fourteen, fifteen, sixteen, seventeen, eighteen, nineteen, or twenty lift attachments sites. In an aspect, all of the one or more lift attachment sites are used to attach the lift garment to a lift apparatus. In some embodiments, only a subset of the one or more lift attachment sites is used to attach the lift garment to a lift apparatus. In some embodiments, the number of lift attachment sites associated with the lift garment is dependent on the size of the intended user. For example, a subject of substantial girth may require a larger lift garment with more lift attachment sites to adequately and/or safely lift the subject. For example, a lift garment designed for a large non-human subject, e.g., a thoroughbred horse, may require a larger lift garment with more lift attachment sites.

In an aspect, at least one of the one or more lift attachment sites is associated with a portion of the fabric-like material worn on a front portion of the subject. For example, one or more lift attachment sites can be appropriately arranged on the front side of an article of clothing designed for a human subject. For example, lift attachment sites can be positioned one an upper chest portion of the lift garment and the hip portion of the lift garment. In some embodiments, the one or more lift attachment sites are associated with a back portion of the fabric-like material of the lift garment. For example, one or more lift attachment sites can be appropriately arranged on the back side of a lift garment designed for a thoroughbred horse or other large animal.

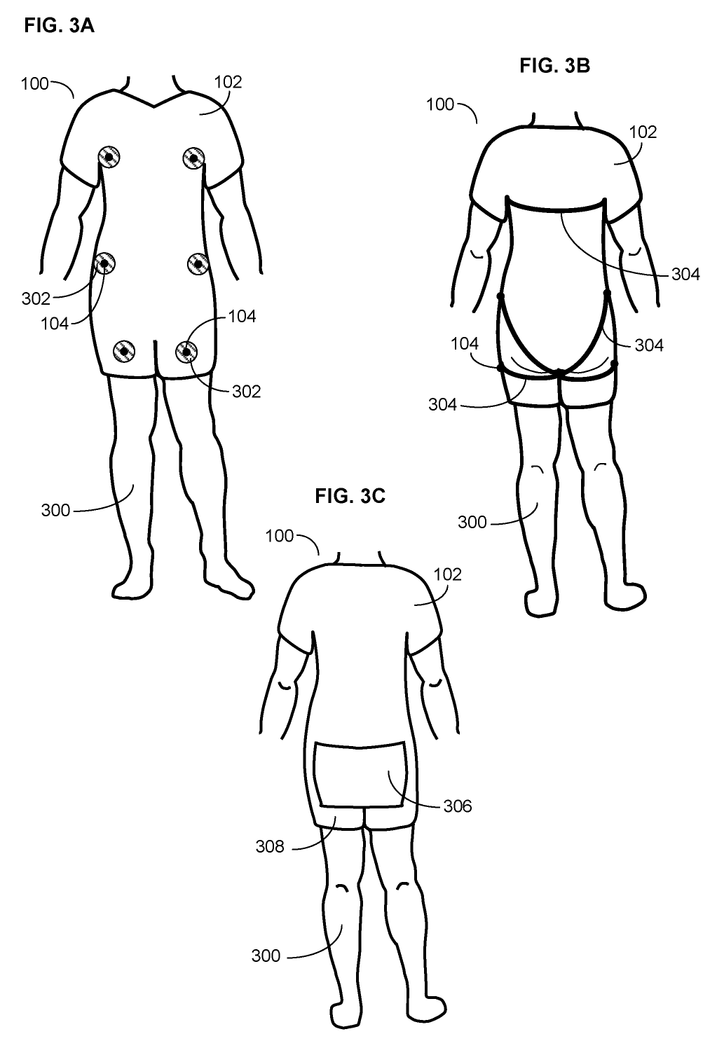

In an aspect, lift garment 100 further includes a first reinforcing material attached to or incorporated into at least one of the one or more lift attachment sites 104. FIG. 3A illustrates a non-limiting example of a lift garment 100 worn by subject 300 and including fabric-like material 102 and a first reinforcing material 302 associated with lift attachment sites 104. For example, the fabric-like material of the lift garment can include a reinforcing material, e.g., a fabric, textile, or other sheet of material having increased tensile strength, which is used to reinforce the lift attachment sites where the lift attachment elements are incorporated. In some embodiments, the first reinforcing material 302 is adhered to or sewn on at least one surface of the fabric-like material 102 at the at least one of the one or more lift attachment sites 104. For example, a thick canvas material can be used to surround an opening including a grommet or eyelet for use in attaching the lift garment to the lift apparatus. In some embodiments, the first reinforcing material 302 is incorporated into the fabric-like material 102 at the at least one of the one or more lift attachment sites 104. For example, a thread or yarn with increased tensile strength can be woven or knit into the fabric-like material at the one or more lift attachment sites.

In an aspect, a lift garment includes at least one load path 110 between the one or more lift attachment sites. In an aspect, the at least one load path between the one or more lift attachment sites is a portion of the fabric-like material experiencing a load when a subject wearing the lift garment is attached to and suspended from the lift apparatus. For example, a lift garment including a single lift attachment site positioned, for example, on the front side of the lift garment at mid-torso, may experience a load path along the backside of the lift garment when the lift garment is worn by a subject and attached to a lift apparatus. For example, a lift garment including two or more lift attachment sites may experience multiple load paths between the lift attachment sites depending upon the number and relative positions of the two or more lift attachment sites. In an aspect, the at least one load path 110 between the one or more lift attachment sites 104 is associated with a portion of the fabric-like material worn on a back portion of the subject. For example, the at least one load path between the one or more lift attachment sites can be associated with the backside of a lift garment worn by a human subject. In an aspect, the at least one load path 110 between the one or more lift attachment sites 104 is associated with a portion of the fabric-like material worn on a front or under portion of the subject. For example, the at least one load path between the one or more lift attachment sites can be associated with the underside of a lift garment worn by a large animal, e.g., a horse or cow.

In an aspect, lift garment 100 further includes a second reinforcing material attached to or incorporated into the at least one load path between the one or more lift attachment sites, wherein the second reinforcing material extends along at least a portion of the length of the at least one load path. FIG. 3B illustrates a non-limiting example of a lift garment 100 worn by subject 300 and including fabric-like material 102 and a second reinforcing material 304 attached to or incorporated into at least one load path between lift attachment sites 104. For example, the second reinforcing material can include a thick strap or straps that extend along the length of a load path between two lift attachment sites. For example, the second reinforcing material can include a large patch of fabric, textile, or other flexible material spanning a region where the subject is anticipated to exert a load on the lift garment, e.g., around the buttocks region when the subject is suspended in a seated position. In some embodiments, the second reinforcing material 304 is the same material as the first reinforcing material 302. For example, the first and second reinforcing material can include a fabric, textile, or other flexible material with increased tensile strength relative to the fabric-like material forming the lift garment. In some embodiments, the second reinforcing material 304 is a different material from the first reinforcing material 302. For example, the first reinforcing material can include a fabric, textile, or other flexible material with increased tensile strength relative to the fabric-like material forming the lift garment and the second reinforcing material can include a strap extending along the load path. In an aspect, the at least one load path is associated with a back portion of the fabric-like material 102. For example, in a lift garment designed for a human subject, a second reinforcing material, e.g., a strap, may extend along that portion of the garment that interacts with the lower portion of the buttocks. In an aspect, the second reinforcing material 304 is adhered or sewn to at least one surface of the fabric-like material 102 along the at least a portion of the length of the at least one load path. For example, the second reinforcing material can include at least one strap that is sewn onto the fabric-like material along a load path between one or more lift attachment sites. In an aspect, the second reinforcing material 304 is woven into the fabric-like material 102 along the at least a portion of the length of the at least one load path. For example, a reinforced fiber or metal wire can be woven into the fabric-like material along a load path between one or more lift attachment sites to reinforce the lift garment along the load path.

In an aspect, the lift garment further includes a structural platform associated with the fabric-like material. In an aspect, the structural platform provides structural support to the subject during a lift activity. For example, the structural platform can provide support to the buttocks, back, neck, thighs, shoulders, torso, abdomen, legs or a combination thereof. In an aspect, the lift garment includes a single structural platform. For example, the lift garment can include a single structural platform associated with the seat of the lift garment to provide support to the subject while attached to and/or suspended from a lift apparatus in a seated position. In an aspect, the lift garment includes multiple structural platforms positioned around the lift garment to provide support to the subject. For example, the lift garment can include two or more structural platforms separated from one another by portions of the fabric-like material. In an aspect, the structural platform is formed from a fabric-like material. For example, the structural platform can be formed from a reinforced fabric, a rip-stop fabric, a thick canvas material, or similar materials, non-limiting examples of which have been described above herein. In an aspect, the structural platform is formed from plastic. For example, the structural platform can be formed from a sheet of reinforced plastic, e.g., fiberglass. In an aspect, the structural platform is formed from metal. For example, the structural platform can be formed from a thin sheet of stainless steel.

FIG. 3C illustrates a non-limiting example of a lift garment 100 worn by subject 300 and including fabric-like material 102 and a structural platform 306. In this non-limiting example, the structure platform 306 is positioned to support the buttocks 308 of a subject 300. In an aspect, the structural platform 306 is attached to a surface of the fabric-like material. For example, the structural platform can be at least one of sewn, stapled, glued, pressed, riveted, and/or adhered to a surface of the fabric-like material. In an aspect, the structural platform 306 is insertable into a pocket or pouch associated with the lift garment. For example, the structural platform can include a sheet of material, e.g, plastic or metal, that slips into a pocket or pouch formed with the fabric-like material of the lift garment. In an aspect, the structural platform 306 spans a space between two or more edges of the fabric-like material. For example, the structural platform can be at least one of sewn, glued, pressed, riveted, and/or adhered to two or more edges of the fabric-like material. In an aspect, the structural platform 306 brings the two or more edges of the fabric-like material together to substantially completely encircle the torso and at least a portion of the arms and legs of the subject.

In an aspect, the structural platform 306 includes one or more of the load sensor, the microcontroller with the circuitry, or the reporting device. In an aspect, the structural platform includes a circuit board including sensors, a microcontroller, circuitry and a reporting device. In an aspect, the structural platform 306 includes one or more physiological sensors. For example, the structural platform can include an integrated circuit board including one or more of a heart rate sensor, a blood pressure sensor, a respiration sensor, a temperature sensor, and/or a biochemical sensor. For example, the structural platform can include an integrated circuit board including one or more blood oxygenation sensors.

In some embodiments, a lift garment system includes a lift garment and a removable structural platform, the lift garment including a means for securing the structural platform to the fabric-like material of the lift garment. The means for securing the structural platform can include one or more of a pocket, a pouch, a hook and loop fastener, snaps, an adhesive, straps, and the like for securing the structural platform to the lift garment. In an aspect, the structural platform is removable from the lift garment. For example, the structural platform can be removed to allow for washing/sanitizing of the lift garment. In an aspect, system further includes sensors, a microcontroller with circuitry, and a reporting device associated with the removable structural platform. In an aspect, the system further includes one or more physiological sensors associated with the removable structural platform. In an aspect, the system further includes at least one blood oxygenation sensor associated with the removable structural platform.

### Lift Attachment Elements

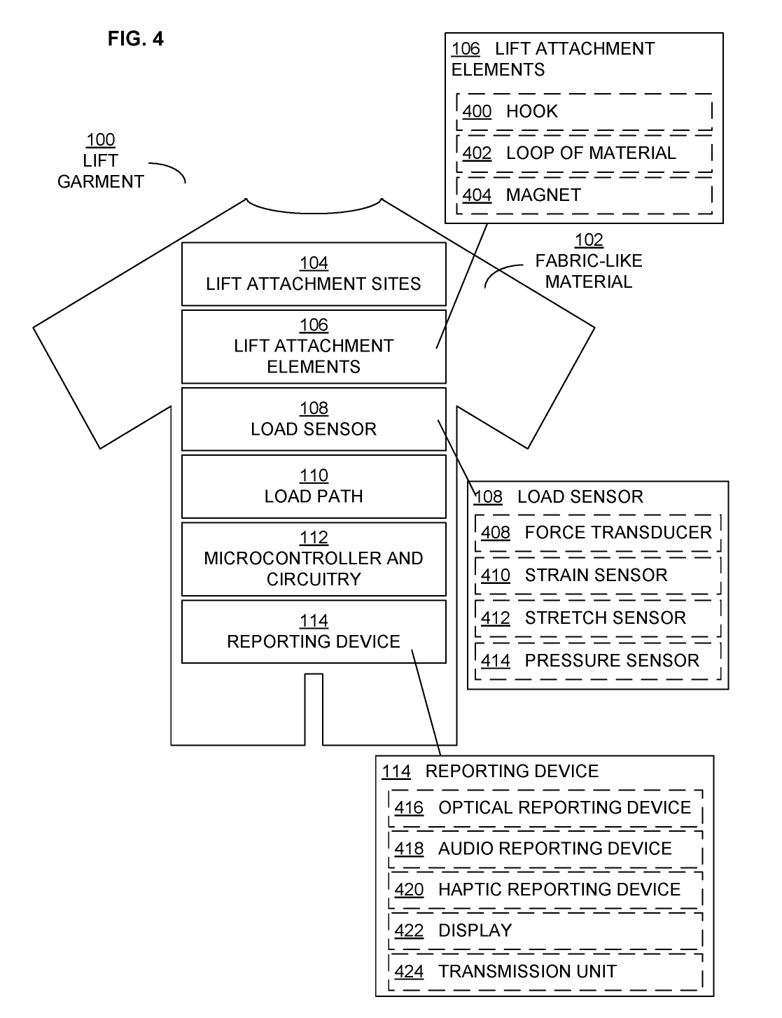

FIG. 4 is a block diagram illustrating further aspects of a lift garment 100. In an aspect, lift garment includes at least one lift attachment element 106 associated with at least one of the one or more lift attachment sites 104. The lift attachment element is configured to attach the lift garment to a lift apparatus. In some embodiments, the at least one lift attachment element 106 includes a hook 400. In some embodiments, the at least one lift attachment element 106 includes a loop of material 402. In some embodiments, the at least one lift attachment element 106 includes a magnet 404.

In some embodiments, each of the one or more lift attachment sites includes a lift attachment element. For example, each of the one or more lift attachment sites can include a hook, a loop of material, or a magnet configured to attach the lift garment to the lift apparatus. In some embodiments, at least a portion of the one or more lift attachment sites includes two or more lift attachment elements. For example, at least a portion of the lift attachment sites can include two or more hooks, loops of material, or magnets configured to attach the lift garment to the lift apparatus.

In some embodiments, a lift attachment element directly attaches a lift garment to a lift apparatus. For example, the lift garment can include a loop of material, e.g., a looped strap, which directly attaches the lift garment to a lift apparatus. For example, the lift garment can include a lift attachment element that directly attaches the lift garment to a spreader bar, a cradle, or a like portion of a lift apparatus. In some embodiments, a lift attachment element indirectly attaches a lift garment to a lift apparatus. For example, the lift attachment element can indirectly attach the lift garment to the lift apparatus through a strap or a chain. For example, a first end of a strap or chain can be attached to a lift attachment element (e.g., a hook) associated with the lift garment and a second end of the strap or chain can be attached to a spreader bar, a cradle, or a like portion of the lift apparatus.

In an aspect, a lift attachment element 106 includes a hook 400. In an aspect, the lift attachment element includes a closable hook, e.g., a bolt-snap hook. For example, the lift attachment element can include a carabiner or similar closable hook. In an aspect, the lift attachment element includes a clip hook. In an aspect, the lift attachment element can include two or more components. For example, the lift attachment element can include a grommet-reinforced opening in the fabric-like material and a hook, e.g., a double-end bolt-snap hook, or a clip. In an aspect, a hook associated with the lift garment hooks directly onto a spreader bar or cradle of a lift apparatus. In an aspect, a hook associated with the lift garment hooks to a strap or chain connected to the spreader bar or cradle of the lift apparatus.

In an aspect, a lift attachment element 106 includes a loop of material 402. In an aspect, the loop of material is an extension of the fabric-like material forming the lift garment. In an aspect, the loop of material is a piece of material, e.g., strapping, that is attached to the fabric-like material of the lift garment. In an aspect, the loop of material is a loop of metal, plastic, or rubber material attached to the fabric-like material. For example, the loop of material can include a metal, plastic, or rubber ring or similar structure attached (e.g., sewn or glued) to the fabric-like material of the lift garment. In an aspect, the loop of material of the lift garment is configured to attach to a spreader bar(s) or cradle(s) of lift apparatus. In an aspect, the loop of material of the lift garment is configured to attach to one or more hooks or clips associated with the spreader bar(s) or cradle(s) of the lift apparatus.

In an aspect, a loop of material 402 includes an opening defined by the fabric-like material of the lift garment. For example, the lift attachment element can include a hole in the fabric-like material of the lift garment. In an aspect, a loop of material 402 includes an opening defined by a first reinforcing material attached to or incorporated into the fabric-like material at the one or more lift attachment sites of the lift garment. For example, the lift attachment element can include a reinforced hole in the fabric-like material of the lift garment. In an aspect, a loop of material 402 includes an opening defined by the fabric-like material of the lift garment and reinforced with a grommet. For example, the lift garment can include one or more grommets inserted into one or more holes in the fabric-like material of the lift garment. In an aspect, the opening defined by the fabric-like material is reinforced with a circular grommet. In an aspect, the opening defined by the fabric-like material is reinforced with a square or rectangular grommet. In an aspect, the opening defined by the fabric-like material is reinforced with a grommet made from at least one of metal, metal alloy, rubber, or plastic. For example, the grommet can be formed from brass, nickel, stainless steel, or other metal or metal alloy. In some embodiments, the lift garment includes a tab of reinforced fabric-like material including an opening reinforced with a grommet. In an aspect, the grommet associated with the lift garment attaches the lift garment to the lift apparatus through a chain or strap with a hook or clip that can be inserted through the grommet.

In an aspect, a lift attachment element 106 includes a magnet 404. For example, one or more magnets can be sewn into a portion of the fabric-like material at the one or more lift attachment sites. In an aspect, a magnet associated with the lift garment directly connects with a magnetized spreader bar, cradle, or portion thereof of a lift apparatus. In an aspect, a magnet associated with the lift garment connects to a magnetized strap or chain connected to the spreader bar or cradle of the lift apparatus.

In an aspect, the lift attachment element includes a snap, a buckle, a clamp, a button, a clasp, a cable tie, a clip, hook and loop fastener, latch, pin, twist tie, or zipper. In some embodiments, the lift attachment element is combined with one or more other lift attachment elements. For example, the lift attachment element might include a loop of material through which a hook (e.g., a clip hook) has been attached. In some embodiment, the lift attachment element is associated with a strap, a chain, a rope, or similar structures intended to span a distance between the lift garment and an attachment site (e.g., a spreader bar or cradle) on the lift apparatus. For example, the lift garment can include one or more straps including a hook or loop of material at the end of each strap for use in attaching the lift garment to a lift apparatus.

The lift attachment elements are configured to attach to a lift apparatus. In an aspect, the lift attachment elements are configured to attach to a mobile (or floor) lift apparatus. In an aspect, the lift attachment elements are configured to attach to a Hoyer lift apparatus. In an aspect, the lift attachment elements are configured to attach to any of a number of other free standing lift apparatuses. For example, the lift attachment elements can be configured to attach to a patient hoist, jack hoist, or hydraulic lift. In an aspect, the lift attachment elements are configured to attach to a stationary lift apparatus. For example, the lift attachment elements can be configured to attach to a lift apparatus incorporated into or mounted on the ceiling or one or more walls of a subject's room (e.g., a room in a residence, a hospital, or skilled nursing facility).

### Load Sensors

Returning to FIG. 4, lift garment 100 further includes a load sensor 108 associated with at least one of the one or more lift attachment sites 104 or along at least one load path 110 between the one or more lift attachment sites 104. In an aspect, the load sensor 108 includes a force transducer 408. In an aspect, the load sensor 108 includes a strain sensor 410. In an aspect, the load sensor 108 includes a stretch sensor 412. In an aspect, the load sensor 108 includes a pressure sensor 414.

In an aspect, a lift garment includes a load sensor associated with at least one of the one or more lift attachment sites. In an aspect, a lift garment includes a load sensor associated with each of the one or more lift attachment sites. In an aspect, the load sensor is associated with the interface between the lift attachment site and the lift attachment element. In an aspect, a load sensor associated with the one or more lift attachment sites is configured to measure the force exerted on a portion or portions of the lift garment when worn by a subject suspended from a lift apparatus during a lifting or transfer procedure. In some embodiments, the load includes the force exerted on the lift garment by at least a portion of the weight of the subject during a lifting procedure. In some embodiments, the load includes the force exerted by the lift garment on the subject during a lifting procedure. In some embodiments, the load includes the force exerted on the one or more lift attachment sites. For example, the load can include the force exerted at each of the one or more lift attachment sites as a subject wearing the lift garment is suspended from the lift apparatus.

In some embodiments, the load carried (and measured) at any given lift attachment site is proportional to the total number of lift attachment sites engaged with the lift apparatus and supporting the weight of the suspended subject. For example, if there is one lift attachment site engaged with the lift apparatus, than the full weight and associated load exerted by the subject on the lift garment will be experienced at that one lift attachment site. Conversely, if two or more lift attachment sites of the lift garment are engaged with the lift apparatus, the weight and associated load exerted by the subject on the lift garment will be experienced proportionally at the two or more lift attachment sites. However, depending upon how the subject is positioned in the lift garment or how the lift garment is attached to the lift apparatus, the load at any given lift attachment site may vary. For example, if the center of gravity of the subject is not centered over the lift point, one lift attachment site could experience more load than another, leading to instability during the lifting procedure.

In an aspect, the load sensor is associated with a load path between one or more lift attachment sites. In an aspect, a load sensor associated with the at least one load path between the one or more lift attachment sites is configured to measure the force exerted on a portion or portions of the lift garment when worn by a subject suspended from a lift apparatus. For example, the load can include the force exerted along a portion of the fabric-like material between the one or more lift attachment sites. In an aspect, two or more load sensors are distributed along a length of the at least one load path between the one or more lift attachment sites.

In some embodiments, the load carried (and measured) at any given lift attachment site or along a load path between one or more lift attachment sites depends upon the angle between the lift attachment site/lift attachment element and the actual attachment site on the lift apparatus. In some embodiments, the more vertical the lift attachment site/lift attachment elements are upon attachment to the spreader bar(s) of a lift apparatus, the less overall load per attachment point.

In an aspect, the at least one load sensor includes a force transducer. Non-limiting examples of force transducers include strain gauge load cells, piezoelectric crystal, quartz force transducers, linear variable differential transducer (LVDT), capacitive load cells, tuning fork load cells, vibrating wire transducer, gyroscopic, or force balance.

In an aspect, the load sensor includes a strain sensor. For example, the load sensor can include a strain gauge. Non-limiting examples of strain gauge load cells include semiconductor gauges, thin film gauges, wire strain gauge, optical strain gauge, and foil gauges. In some embodiments, the strain gauge takes advantage of electrical conductance and the geometry of the conductor and when stretched within the limits of its elasticity, it will become narrower and longer causing measurable changes in its electrical resistance end-to-end. From the measured electrical resistance of the strain gauge, the amount of induced stress may be inferred. See, e.g., U.S. Pat. No. 6,360,615 to Smela titled “Wearable effect-emitting strain gauge devices,” which is incorporated herein by reference. In an aspect, the strain gauge is incorporated into the fabric-like material forming the lift garment. See, e.g., Mattman et al. (2008) “Sensor for measuring strain in textile,” Sensors 8:3719-3732, which is incorporated herein by reference.

In an aspect, the load sensor includes a stretch sensor. In some embodiments, the stretch sensor is incorporated into the fabric-like material. For example, the stretch sensor can include a pattern of conductive wire or yarn woven or knit into the fabric-like material to form a stretch sensor. In some embodiments, the stretch sensor is attached to or sewn on the fabric-like material. For example, the stretch sensor can include a commercially available stretch sensor, non-limiting examples of which include a fabric stretch sensor with a sewable zone from StretchSense, Aukland, New Zealand, conductive rubber cord stretch sensors from Adafruit, New York City, N.Y.; Tactilus® Stretch, Sensor Products Inc., Madison, N.J.

In an aspect, the load sensor includes a pressure sensor. For example, the load sensor can include one or more individual pressure sensors attached to or incorporated into the fabric-like material of the lift garment. Tactile-type pressure sensors are available from commercial sources from, for example, StretchSense, Aukland, New Zealand or Sensor Products Inc., Madison, N.J. See, e.g., Buscher et al. (2015) “Flexible and stretchable fabric-based tactile sensor,” Robotics Autonomous Systems 63:244-252, which is incorporated herein by reference. In an aspect, the load sensor is part of a sensor array including a plurality of pressure sensors. In an aspect, the load sensor is a sensor array distributed along the length of at least one load path between the one or more lift attachment sites. For example, the load sensor can include a pressure array distributed over a back portion of the lift garment that measures the pressure, i.e., interaction, between that portion of the lift garment and a body portion of the subject. It is anticipated that the pressure between the lift garment and the body portion of the subject will increase as the lift garment attached to the lift apparatus takes on the weight of the subject. In an aspect, the load sensor can include an array of pressure sensors distributed over a portion of the lift garment, e.g., over that portion of the lift garment covering the buttocks of the subject. A non-limiting examples of a pressure array sensor is provided by the LX100×3 sensor array from XSENSOR® Technology Corporation, Calgary, Canada or the Stretchable TactArray Sensor from Pressure Profile Systems, Los Angeles, Calif.

In an aspect, the load sensor is associated with the fabric-like material. In an aspect, the load sensor is at least one of embroidered, sewn, woven, knitted, spun, breaded, coated/laminated, or printed onto or into the fabric-like material of the lift garment. In an aspect, the load sensor is attached to the fabric-like material at one or more lift attachment sites and/or along a load path between two lift attachment sites. For example, a commercially available stretch sensor or pressure sensor can be sewn onto the lift garment. In an aspect, the load sensor is incorporated (e.g., woven or knitted) into the fabric-like material at one or more lift attachment sites. For example, components of a strain gauge can be woven into the fabric-like material using conductive fiber, thread, or yarn. In an aspect, the load sensor is sewn into the fabric-like material. For example, a pattern forming a load sensor can be sewn into or onto the fabric-like material with conductive yarn, thread, and/or wire. In an aspect, the load sensor is printed onto a surface of the fabric-like material. For example, a pattern of conductive ink (containing, for example, silver, copper, or gold nanoparticles) forming the load sensor can be screen-printed onto a surface of the formed fabric-like material. Non-limiting examples of stretch and pressure sensors associated with textiles are described in Stoppa and Chiolerio (2014) “Wearable electronics and smart textiles: A critical review,” Sensors 14:11957-11992, which is incorporated herein by reference.

### Microcontroller and Circuitry

Lift garment 100 includes microcontroller 112. The microcontroller includes circuitry configured to receive and process information regarding the measured load from the load sensor. The microcontroller can include a microprocessor, a central processing unit (CPU), a digital signal processor (DSP), application-specific integrated circuit (ASIC), a field programmable gate entry (FPGA), or the like, or any combinations thereof, and can include discrete digital or analog circuit elements or electronics, or combinations thereof. In an aspect, the microcontroller includes one or more ASICs having a plurality of predefined logic components. In an aspect, the microcontroller includes one or more FPGAs having a plurality of programmable logic commands. In an aspect, the microcontroller includes an ASIC chip, an ARM chip, or a programmable logic controller (PLC). The microcontroller can further include signal processing algorithms, e.g., band pass filters, low pass filters, or any other single processing algorithms or combinations thereof.

The microcontroller further includes some form of accessible memory. In an aspect, the microcontroller includes RAM (volatile memory) for data storage. In an aspect, the microcontroller includes ROM, EPROM, EEPROM, or flash memory for program and operating parameter storage. The memory component can be used to store algorithms, subject data, and reference range data, e.g., a range of acceptable load values, a range of acceptable physiological parameter values, or a range of acceptable oxygen saturation levels. The microcontroller further includes in/out (I/O) ports for receiving information, e.g., signals from one or more sensors, and transmitting information, e.g., signals to the reporting device. In an aspect, the microcontroller further includes a clock generator, analog-to-digital convertors, serial ports, and/or data bus to carry information. In an aspect, the microcontroller includes a small integrated chip attached to or incorporated into the lift garment.

In some embodiments, microcontroller 112 includes a stored range of acceptable load values and circuitry configured to determine if the measured load falls within the range of acceptable load values. The stored range of acceptable load values can be specific to the subject and the various load points or paths associated with the lift garment when worn by the subject and attached to a lift apparatus. The stored range of acceptable load values can be specific to the lift garment, e.g., a small, medium, large, extra-large, plus sized lift garment. The stored range of acceptable load values can be specific to a shape of the lift garment. The stored range of acceptable load values can be specific to the number of lift attachment sites and/or distribution of load paths between the one or more lift attachment sites.

In embodiments, a lift garment 100 includes a power source configured to provide power to one or more components of the lift garment including, but not limited to, one or more sensor types, the microcontroller, and/or the reporting device. In an aspect, the power source includes a wired connection to a standard electrical outlet. In an aspect, the power source is associated with the lift apparatus to which the lift garment worn by the subject is attached. In an aspect, the power source is a resident device component associated with the lift garment. Non-limiting examples of resident device components include batteries (e.g., a camera or watch-sized alkaline, lithium, or silver-oxide battery, a thin film battery, a microbattery) and solar cells (e.g., silicon-based solar cells) configured to convert light energy into electrical energy for use by components of the lift garment. In an aspect, the power source includes one or more components positioned remotely from the lift garment that transmit power signals via associated wireless power methods including, but not limited to, inductive coupling of power signals. In an aspect, the lift garment receives power through an energy harvesting unit capable of converting received electromagnetic energy into electrical energy. For example, the lift garment can receive power through energy harvesting from body heat, breathing, or body movement (e.g., walking).

### Reporting Device

With reference to FIG. 4, lift garment 100 further includes reporting device 114 operably coupled to microcontroller 112 and configured to transmit one or more signals indicative of the processed information regarding the measured load. In an aspect, reporting device 114 includes optical reporting device 416. In an aspect, reporting device 114 includes audio reporting device 418 including at least one speaker. In an aspect, reporting device 114 includes haptic reporting device 420. In an aspect, reporting device 114 includes display 422. In an aspect, reporting device 114 includes transmission unit 424.

In an aspect, reporting device 114 is operably coupled to the microcontroller 112 and configured to generate one or more communication signals based on the information regarding the measured load. In an aspect, the reporting device 114 is configured to generate one or more communication signals indicating that the measured load falls within a range of acceptable load values. In an aspect, the reporting device 114 is configured to generate one or more communication signals indicating that the measured load fails to fall within the range of acceptable load values.

In an aspect, the reporting device 114 includes an optical reporting device 416. In an aspect, the optical reporting device 416 includes one or more light indicators. For example, the reporting device can include one or more lights, e.g., light-emitting diodes (LEDs), configured to light up in response to the information regarding the measured load. In an aspect, the optical reporting device 416 includes one or more color-coded lights. For example, the reporting device can include LEDs of different colors and a coding system. For example, a signal from a green LED can indicate a measured load which falls within a range of acceptable load values while a red LED can indicate a measured load that fails to fall within the range of acceptable load values. Sewn-on washable LEDs designed for use with fabric are commercially available (from, e.g., SparkFun Electronics, Niwot, Colo.).

In an aspect, the reporting device 114 includes an audio reporting device 418 including at least one speaker. For example, the reporting device can include an audio reporting device that emits an audible signal in response to the information regarding the measured load. For example, the audio reporting device can emit a warning sound, e.g., a beeping sound, if the measured load fails to fall within a range of acceptable load values. For example, the audio reporting device can emit a spoken words indicating whether the measured load falls within the range of acceptable load values. Electronic sound chips and/or sound cards are available from commercial sources (from, e.g., STMicroelectronics, Geneva, Switzerland).

In an aspect, the reporting device 114 includes a haptic reporting device 420. For example, the reporting device can include a haptic reporting device that emits a haptic signal, e.g., a vibrational signal, in response to the information regarding the measured load. In an aspect, the haptic reporting device is incorporated into or onto a surface of the lift garment that is in direct contact with the subject wearing the lift garment. For example, the haptic reporting device can include a vibrational motor (e.g., a coin or pancake vibration motor, from, e.g., Precision Microdrives Ltd, London, UK) might be used as a warning system for a subject wearing the lift garment who is operating a lift apparatus on their own.

In an aspect, the reporting device 114 includes a display 422 configured to report, communicate or otherwise provide information to a user, e.g., the subject, an attendant, or healthcare provider. The display can include, but is not limited to, a graphical user interface, a touchscreen assembly (e.g., a capacitive touch screen), a liquid crystal display (LCD), or a light-emitting diode (LED) display. For example, a display can include a flexible, flat LCD attached to or incorporated into the lift garment. See, e.g., U.S. Pat. No. 5,912,653 to Fitch titled “Garment with programmable video display unit,” which is incorporated herein by reference. For example, the reporting device can include an LED display attached to or incorporated into the lift garment. For example, the reporting device can include a small ultrathin OLED (organic light-emitting diode) display attached to or incorporated into the lift garment. See, e.g., Cochrane et al. (2011) “Flexible displays for smart clothing: Part 1—Overview,” Indian J. Fibre & Textile Res., 36:422-428, which is incorporated herein by reference.

In an aspect, the reporting device 114 includes a transmission unit 424 including an antenna. A “transmission unit,” as used herein, can be one or more of a variety of units that are configured to send and/or receive signals, such as signals carried as electromagnetic waves. A transmission unit generally includes at least one antenna and associated circuitry. A transmission unit can be operably connected to the microcontroller and/or can include its own a processor and/or memory component. A transmission unit can be operably connected to an energy source, such as a battery. A transmission unit can include an energy harvesting unit, such as a unit configured to obtain energy from electromagnetic waves. A transmission unit can include a transponder utilizing electromagnetic waves, for example as described in “Fundamental Operating Principles,” in Chapter 3 of the RFID Handbook: Fundamentals and Applications in Contactless Smart Cards and Identification, Klaus Finkenzeller, John Wiley & Sons, (2003), which is incorporated herein by reference. A transmission unit can include an oscillator and encoder configured to generate a programmable pulse position-modulated signal in the radio frequency range (see, e.g., U.S. Pat. No. 4,384,288, which is incorporated herein by reference). A transmission unit can include a radio frequency identification device (RFID), which can be a passive RFID device, a semi-passive RFID device, or an active RFID device, depending on the embodiment (see, e.g., Chawla & Ha, “An Overview of Passive RFID,” IEEE Applications and Practice, 11-17 (September 2007), which is incorporated herein by reference). A transmission unit including an RFID device can be configured to transmit signals in the UHF standard range. A transmission unit can include a battery-assisted passive RFID device, such as sold by Alien Technology®, Morgan Hill, Calif. A transmission unit can include an optical transmission unit. A transmission unit can include a hybrid backscatter system configured to function in an RFID, IEEE 802.11x standard and Bluetooth system (see, e.g., U.S. Pat. No. 7,215,976, which is incorporated herein by reference). A transmission unit can include a near field communication (NFC) device. A transmission unit can include a Wireless Identification and Sensing Platform (WISP) device. A transmission unit can be operably coupled to a data storage unit, for example as described in U.S. Pat. No. 7,825,776 and US Patent Application No. 2009/0243813, which are each incorporated herein by reference.

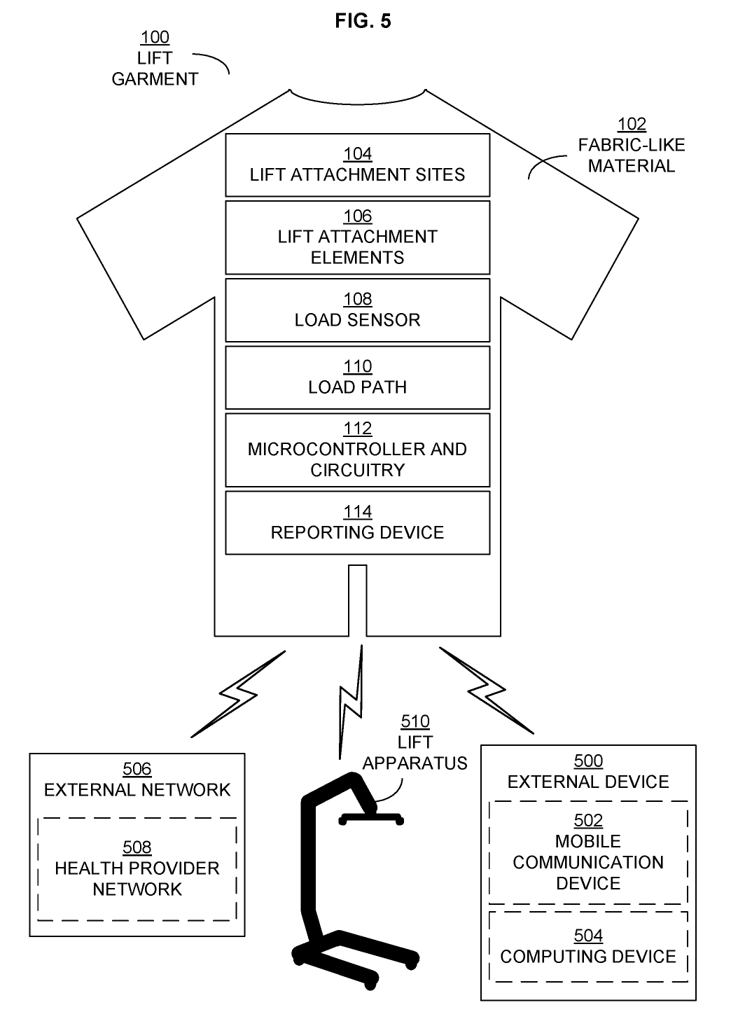

FIG. 5 shows further aspects of a lift garment. In an aspect, reporting device 114 of lift garment 100 is configured to communicate with an external device 500 (e.g., a remote entity, a remote device, a remote server, a remote network, and so forth). For example, the reporting device can include a connection to a computing device or other device configured to accept information from the reporting device. For example, the reporting device can include a wired connection to the lift apparatus. In an aspect, the reporting device 114 is configured to wirelessly communicate with the external device 500. The reporting device can communicate via one or more connected and wireless communication mechanisms including, but not limited to, acoustic communication signals, optical communication signals, radio communication signals, infrared communication signals, ultrasonic communication signals, and the like. For example, the reporting device can include a transmission unit including a radio antenna configured to wirelessly communicate with an external device. In an aspect, reporting device 114 (e.g., a transmission unit) is configured to transmit information regarding the measured load or other measured parameter (e.g., a physiological parameter or oxygen saturation) to a computing component, e.g., a personal computing device or a laptop computing device. For example, the transmission unit can be configured to transmit information regarding the measured load or other measured parameter (e.g., a physiological parameter or oxygen saturation) to a remote computing device, e.g., a remote computing device associate with a website, the Internet, or the Cloud.

In an aspect, the reporting device 114 is configured to communicate with the lift apparatus 510. For example, the reporting device can communicate (e.g., transmit one or more signals) to the lift apparatus to control a function of the lift apparatus based on the processed information regarding the measured load. For example, the reporting device can transmit one or more signals to the lift apparatus to control at least one of an on/off, up/down, speed, or acceleration function or operation of the lift apparatus.

In an aspect, the reporting device 114 is configured to communicate with a mobile communication device 502. For example, the external device can include a smart phone or other mobile communication device. In an aspect, the mobile communication device 502 includes a program, set of instructions, and/or application configured to receive information from the lift garment, process the information, and display the information for a user. In an aspect, the reporting device 114 is configured to communicate with a computing device 504. For example, the external device can include a tablet, laptop, or desktop computing device. In an aspect, the external device 500 includes a communication device, such as one or more of a mobile communication device and a computer system including, but not limited to, mobile computing devices (e.g., hand-held portable computers, Personal Digital Assistants (PDAs), laptop computers, netbook computers, tablet computers, and so forth), mobile telephone devices (e.g., cellular telephones and smartphones), devices that include functionalities associated with smartphones and tablet computers (e.g., phablets), portable game devices, portable media layers, multimedia devices, satellite navigation devices (e.g., Global Positioning System (GPS) navigation devices), e-book reader devices (eReaders), Smart Television (TV) devices, surface computing devices (e.g., table top computers), Personal Computer (PC) devices, and other devices that employ touch-based human interfaces. In an aspect, the computing device is associated with the lift apparatus. In an aspect, the computing device is associated with another piece of equipment associated with a patient care room in a hospital, skilled nursing, or assisted living facility.

In an aspect, reporting device 114 is configured to communicate with an external network 506. In an aspect, reporting device 114 is configured to communicate with a health provider network 508. For example, the reporting device can be configured to communicate directly with a network associated with a subject's healthcare provider, e.g., a hospital, a clinic, medical facility, or physician's office. For example, the reporting device can be configured to communicate directly with the subject's electronic medical file or health record.

### Load Limit Label

In an aspect, a lift garment includes at least one load limit label. In an aspect, the load limit label provides a user with a load limit for the lift garment, one or more lift attachments sites, one or more lift attachment elements, and/or at least one load path between one or more lift attachment sites. In an aspect, the load limit label may be centrally located on the lift garment and include information regarding load limits for a lift garment, one or more lift attachments sites, one or more lift attachment elements, and/or at least one load path between one or more lift attachment sites. In an aspect, a load limit label is associated with at least one of the one or more lift attachment sites. In an aspect, a load limit label is associated with at least one load path between the one or more lift attachment sites. In an aspect, a load limit label is associated with at least one of the lift attachment elements.

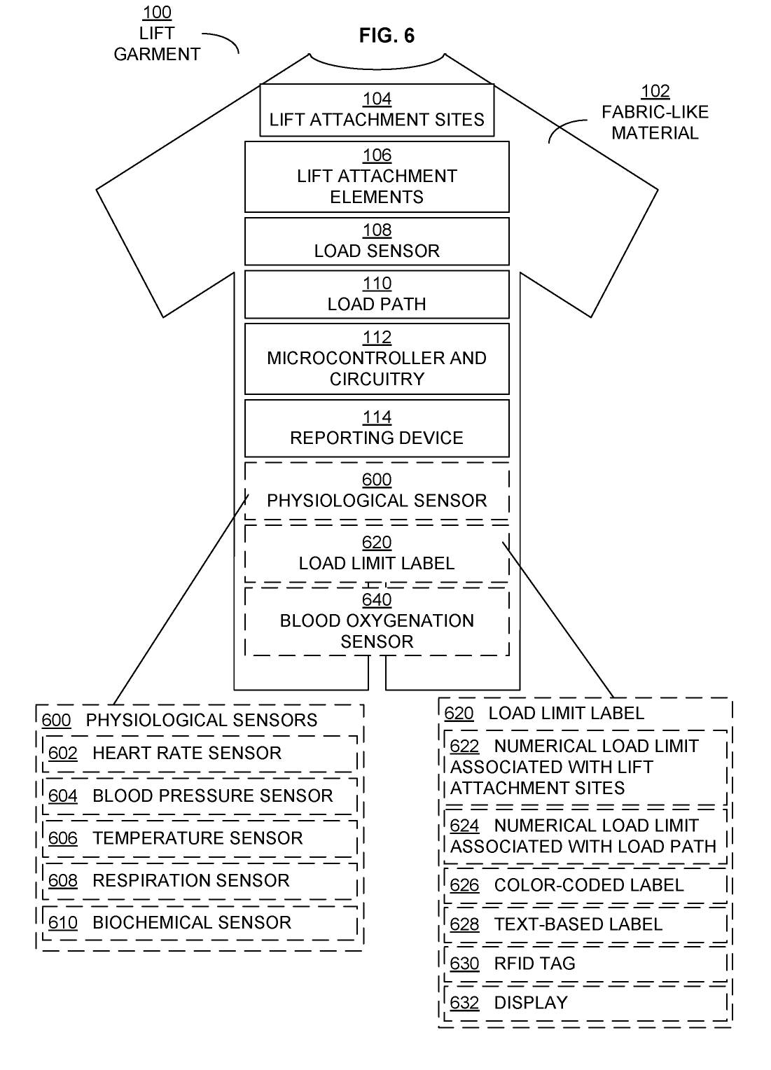

FIG. 6 illustrates aspects of lift garment 100 including at least one load limit label 620. In an aspect, the at least one load limit label 620 includes in block 622 a numerical load limit associated with each of the one or more lift attachment sites 104. In an aspect, the at least one load limit label 620 includes in block 624 a numerical load limit associated with the at least one load path 110 between the one or more lift attachment sites 104. In an aspect, the at least one load limit label 620 includes a color-coded label 626. For example, the color-coded label can indicate the load limit, e.g., maximum weight of a subject, for use of the lift garment. As a non-limiting example, a blue label could indicate a 250 pound maximum weight, a green label could indicate a 150 pound maximum weight, and a red label could indicate a 130 pound maximum weight. In an aspect, the at least one load limit label 620 includes a text-based label 628. For example, the load limit label can include a series of readable text and/or numbers describing the load limits at any given place(s) associated with the lift garment. In an aspect, the at least one load limit label 620 includes an electronic label. In an aspect, the at least one load limit label 620 includes a radiofrequency identification (RFID) tag 630. For example, information regarding the load limit(s) associated with a lift garment can be stored on RFID tags associated with the lift garment and read by an RFID reader. Sew-on waterproof RFID tags are available from commercial sources (for example, Logi Tag® RFID tags from HID Global Corporation, Austin, Tex.). Alternatively, the RFID tag can include a passive chipless RFID tag sewn directly into the fabric-like material of the lift garment using conductive thread. See, e.g., Vena et al. (2013) “Design and realization of stretchable sewn chipless RFID tags and sensors for wearable applications,” IEEE: International Conference of RFID 2013; DOI: 10.1109/RFID.2013.6548152. In an aspect, the at least one load limit label 620 includes a display 632. For example, the display can include an LCD display. For example, the display can include an LED or OLED display. In some embodiments, a display can be used as both a reporting device and a load limit label to provide information to a user. Non-limiting aspects of displays have been presented above herein.

### Physiological Sensors

In an aspect, a lift garment further includes one or more physiological sensors configured to measure at least one physiological parameter of the subject. FIG. 6 illustrates aspects of lift garment 100 including one or more physiological sensors 600. In an aspect, at least one of the one or more physiological sensors 600 includes a heart rate sensor 602. In an aspect, at least one of the one or more physiological sensors 600 includes a blood pressure sensor 604. In an aspect, at least one of the one or more physiological sensors 600 includes a temperature sensor 606. In an aspect, at least one of the one or more physiological sensors 600 includes a respiration sensor 608. In an aspect, at least one of the one or more physiological sensors 600 includes a biochemical sensor 610.

In some embodiments, the one or more physiological sensors 600 include at least one heart rate sensor 602. For example, the heart rate sensor can include a commercially available pulse sensor from, e.g., SparkFun. In an aspect, the heart rate sensor includes an optical based heart rate sensor. For example, the heart rate sensor can include a fabric-based sensor (e.g., Textro-Sensors® from Textronics® Inc., Chadds Ford, Pa.) that uses changes in light transmission and reflection to measure changes in stretch of the fabric in response to heart beat and/or respiration. For example, the heart rate sensor can include a form of photoplethsymography. See, e.g., Shyamkumar et al. (2014) “Wearable wireless cardiovascular monitoring using textile-based nanosensor and nanomaterial systems,” Electronics 3:504-520, which is incorporated herein by reference. In an aspect, the heart rate sensor includes at least one electrode. In an aspect, the lift garment can include a series of ECG electrodes (e.g., 10 electrodes) distributed at key points in the lift garment (e.g., on each arm, on each leg, and several on the torso in proximity to the heart). In an aspect, the ECG electrodes are able to detect various amplitudes and intervals of a heart beat (e.g., P wave, PR interval, QRS complex, J point, ST segment, T wave, QT interval, and U wave). In an aspect, the ECG electrodes are also able to detect ischemia or infarction events based on changes in the amplitudes and intervals of a heartbeat. In some embodiments, the ECG electrodes are printed onto the fabric-like material of the lift garment with conductive ink. See, e.g., US 2015/0250420 to Longinotti-Buitoni et al. titled “Physiological Monitoring Garment,” which is incorporated herein by reference. In some embodiments, the ECG electrodes are manufactured separately and incorporated into the lift garment. The ECG electrodes are located on an inner surface of the lift garment for contact with the surface of the subject.

In some embodiments, the one or more physiological sensors 600 include at least one blood pressure sensor 604. In an aspect, a blood pressure sensor in the form of a cuff is incorporated into a sleeve portion of the lift garment. In an aspect, the one or more physiological sensors include one or more ECG electrodes from which blood pressure can be estimated. See, e.g., Shyamkumar et al. (2014) “Wearable wireless cardiovascular monitoring using textile-based nanosensor and nanomaterial systems,” Electronics 3:504-620, which is incorporated herein by reference.

In an aspect, the one or more physiological sensors 600 includes at least one temperature sensor 606. The temperature sensor is configured to measure a surface temperature of the subject. Surface temperature probes are available from commercial sources (from, e.g., Measurement Specialties™, Hampton, Va.; Smiths Medical, St. Paul, Minn.). In an aspect, the temperature sensor includes an infrared emitter-detection system. In an aspect, the temperature sensor is in direct contact with the surface of the subject, e.g., a skin surface of the subject. For example, the temperature sensor can include an optical fiber grating temperature sensor, a non-limiting example of which is described by Li et al. (2012) “Wearable sensors in intelligent clothing for measuring human body temperature based on optical fiber Bragg grating,” Optic Express, 20:11740-11752, which is incorporated herein by reference. In an aspect, the temperature sensor measures radiant heat emitted by the subject.

In some embodiments, the one or more physiological sensors 600 include at least one respiration sensor 608. In an aspect, the respiration sensor includes a sensor configured to measure movement of the chest during breathing as a measure of respiration rate (plethysmography). For example, the respiration sensor can include a stretch sensor or strain gauge, a component of which at least partially encircles the chest of the subject and expands and contracts as the subject breaths. For example, the respiration sensor can include a strain gauge formed with stretchable conductive ink printed onto a portion of the fabric-like material corresponding to a portion of the subject's torso (e.g., at the level of the xiphoid process and/or the belly button). In an aspect, the respiration sensor includes piezoresistive textile patch that measures the expansion of the chest during respiration. In an aspect, the respiration sensor can include a microphone configured to measure sounds made by the subject while breathing as a measure of respiration rate. In an aspect, the respiration sensor includes a sensor configured to measure expired breath as a measure of respiration rate. For example, the respiration sensor can include a chemical sensor for sensing expired volatile organic compounds in the expired breath. For example, the respiration sensor can include a humidity sensor or a temperature sensor that measures localized changes in humidity or temperature associated with the expired breath.

In some embodiments, the one or more physiological sensors 600 include at least one biochemical sensor 610. In an aspect, the biochemical sensor is configured to measure a biochemical analyte associated with the subject. In an aspect, the biochemical sensor is configured to measure a biochemical analyte associated with the skin, perspiration, or expired breath of the subject. For example, the biochemical sensor can include a pH sensor configured to measure the pH of the subject's perspiration. For example, the biochemical sensor can include an “electronic nose” for measuring volatile organic compounds in the expired breath of the subject. In an aspect, the biochemical sensor is configured to measure a biochemical analyte associated with the blood of the subject. For example, the biochemical sensor can include a glucose monitoring sensor. In an aspect, the biochemical sensor includes an optical sensor, e.g., near-infrared spectroscopy, for determining blood glucose levels using transmittance microscopy. In an aspect, the biochemical sensor includes one or more microneedles configured to collect samples from beneath the skin of the subject.

In an aspect, at least one of the one or more physiological sensors 600 is attached to at least one surface of the fabric-like material 102. For example, at least one of the physiological sensors can be adhered to a fabric-like material with an adhesive. For example, at least one of the physiological sensors can be attached to the fabric-like material by stitching or sewing. For example, at least one of the physiological sensors can be printed with conductive ink onto a surface of the fabric-like material. In an aspect, at least one of the one or more physiological sensors 600 is incorporated into at least one surface of the fabric-like material 102. For example, at least one of the physiological sensors can be formed by weaving components of the physiological sensors (e.g., conductive wires, fibers, threads, or yarn) into the fabric-like material. In an aspect, at least one of the one or more physiological sensors 600 is woven, knitted, laminated, printed, or stitched into or onto the fabric-like material 102. In an aspect, at least one of the one or more physiological sensors is sewn, embroidered, spun, breaded coated, laminated, or chemical treated into or onto the fabric-like material. Non-limiting examples of physiological sensors associated with textiles are described in US 2015/0250420 to Longinotti-Buitoni et al. titled “Physiological monitoring garments;” Stoppa and Chiolerio (2014) “Wearable electronics and smart textiles: A critical review,” Sensors 14:11957-11992; and Shyamkumar et al. (2014) “Wearable wireless cardiovascular monitoring using textile-based nanosensor and nanomaterial systems,” Electronics 3:504-520, which are incorporated herein by reference.

In an aspect, the lift garment 100 includes one or more secondary sensors. In an aspect, the one or more secondary sensors include environmental sensors configured to measure a parameter in the subject's environment (e.g., temperature, light, and humidity of the subject environment). In an aspect, the one or more secondary sensors include position sensors, e.g., accelerometers, gyroscopes, altimeters, motion sensors, tilt sensors, inclination sensors, and the like configured to measure positional information regarding the subject while seated or supine in the lift garment and attached to the lift apparatus.

In an aspect, the microcontroller 112 includes circuitry configured to receive and process information regarding the measured at least one physiological parameter of the subject; and the reporting device 114 is configured to transmit the processed information regarding the measured at least one physiological parameter of the subject. In some embodiments, the reporting device 114 is configured to transmit the processed information regarding the measured at least one physiological parameter of the subject to an external device. For example, the reporting device can transmit information regarding a measured physiological parameter, e.g., heart beat or blood pressure, to an external device, e.g., a mobile communication device or a computer. In some embodiments, the reporting device 114 is configured to transmit the processed information regarding the measured at least one physiological parameter of the subject to an external network. For example, the reporting device can transmit information regarding a measured physiological parameter, e.g., weight of the subject, to a heath provider network that includes an electronic medical record of the subject.

In some embodiments, the microcontroller 112 includes circuitry configured to receive and process information regarding the measured at least one physiological parameter of the subject; determine whether the measured physiological parameter falls within a range of acceptable physiological parameter values; and transmit a control signal to the lift apparatus to change an operation of the lift apparatus if the measured physiological parameter fails to fall within the range of acceptable physiological parameter values. For example, the microcontroller can include circuitry configured to received and process information from one or more heart rate sensors, determine whether the measured heart rate falls within a range of acceptable heart rates, and stop or lock operation of the lift apparatus if the heart rate of the subject is too high, potentially indicating stress or panic associated with the lifting procedure.

In an aspect, lift garment 100 includes at least one blood oxygenation sensor 640 configured to measure an oxygen saturation level of the subject. In an aspect, the blood oxygenation sensor is configured to measure the oxygen saturation level of the subject's blood. In an aspect, the blood oxygenation sensor is configured to measure the oxygen saturation level of the subject's tissue. In an aspect, the blood oxygenations sensor is configured to measure the peripheral capillary oxygen saturation of the subject. In an aspect, the blood oxygenation sensor includes light-emitting diodes and photodiodes. See, e.g., Voirin (2015) “Working garment integrating sensor applications developed within the PROeTEX project for firefighters,” in Advances in Intelligent Systems and Computing, K. Kinder-Kurlanda and C. Ehrwein Nihan (eds.) Springer International Publishing Switzerland, Vol. 333, pp. 25-33, which is incorporated herein by reference.

In some embodiments, the at least one blood oxygenation sensor 640 is associated with the at least one load path 110. For example, a blood oxygenation sensor can be placed along a load path of the lift garment to monitor blood circulation/supply in the portion of the body proximal to the load path. For example, the blood oxygenation sensor can be used to determine whether a lifting process is cutting off blood supply to one or more extremities, or the like. Normal blood oxygen saturation at sea level and regular room air is between 95 and 100%. Levels under 90 are considered low, resulting in hypoxemia (hypoxia). Blood oxygen levels below 80 percent may compromise organ function, such as brain and heart. In an aspect, the at least one blood oxygenation sensor 640 includes a pulse oximeter. For example, peripheral capillary oxygen saturation which is an estimation of the oxygen saturation level can be measured with a pulse oximeter sensor

In an aspect, the at least one blood oxygenation sensor 640 includes a near infrared optical blood oxygenation sensors. For example, tissue oxygen saturation can be measured by near infrared spectroscopy. See, e.g., Zysset et al. (2013) Textile integrated sensors and actuators for near-infrared spectroscopy,” Optics Express 21:3213-3224, which is incorporated herein by reference. In some embodiments, the at least one blood oxygenation sensor 640 is associated with a surface of the fabric-like material 102 configured for placement in contact with an external surface of the subject. For example, the at least one blood oxygenation sensor can be placed on an internal surface of a garment intended to come in contact with the skin of the subject when worn.

In some embodiments, the microcontroller 112 includes circuitry configured to receive and process information regarding the measured oxygen saturation level of the subject; determine whether the measured oxygen saturation level of the subject falls within a range of acceptable oxygen saturation levels; and transmit a control signal to the lift apparatus to change operation of the lift apparatus if the measured oxygen saturation levels of the subject fails to fall within the range of acceptable oxygen saturation levels. For example, the microcontroller can include circuitry to stop or lock operation of the lift apparatus if the blood oxygenation level of the subject falls precipitously, potentially indicating a cutting of major blood supply while sitting in the lifting procedure.

In some embodiments, a lift garment is part of a system for controlling operation of a lift apparatus. In an aspect, a system includes a lift garment and a lift control mechanism, the lift garment including a fabric-like material shaped to substantially completely encircle at least a portion of a subject's body including a portion of the subject's arms and legs, the fabric-like material including one or more lift attachment sites, at least one lift attachment element associated with the fabric-like material at at least one of the one or more lift attachment sites, the at least one lift attachment element configured to attach the lift garment to a lift apparatus, a load sensor configured to measure a load, the load sensor associated with at least one of the one or more lift attachment sites or along a load path between the one or more lift attachment sites, a microcontroller including circuitry configured to receive and process information regarding the measured load, and a reporting device operably coupled to the microcontroller and configured to transmit one or more signals indicative of the processed information regarding the measured load; and the lift control mechanism including a receiver configured to receive the one or more signals from the reporting device indicative of the processed information regarding the measured load, and circuitry configured control a function of the lift apparatus in response to the one or more signals received from the reporting device.

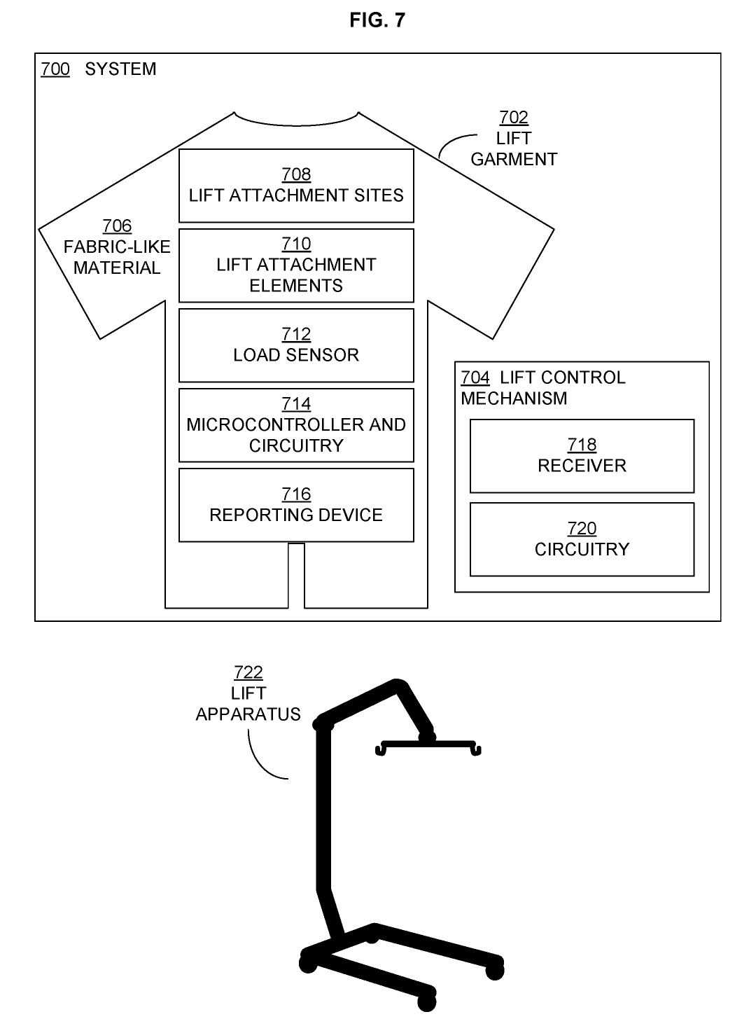

FIG. 7 illustrates aspects of a system 700 including a lift garment 702 and a lift control mechanism 704. Lift garment 702 includes fabric-like material 706 shaped to substantially completely encircle a torso and at least a portion of arms and legs of a subject. For example, the fabric-like material can be shaped as a single article of clothing including short sleeves and pant legs. In some embodiments, lift garment 702 includes fabric-like material 706 shaped to substantially completely encircle the torso and at least a portion of the arms and legs of a human subject. In an aspect, at least a portion of the fabric-like material 706 of lift garment 702 is a woven or a knit material. In an aspect, at least a portion of the fabric-like material 706 of lift garment 702 is a non-woven material. In an aspect, at least a portion of the fabric-like material 706 of lift garment 702 is formed from at least one polymer type. Non-limiting aspects of fabric-like material for use with a lift garment have been described above herein.

Returning to FIG. 7, the fabric-like material 706 of lift garment 702 includes one or more lift attachment sites 708. In some embodiments, the lift garment 702 includes one lift attachment site 708. In some embodiments, the lift garment 702 includes from two to twenty lift attachment sites 708. For example, the lift garment can include two, three, four, five, six, seven, eight, nine, ten, eleven, twelve, thirteen, fourteen, fifteen, sixteen, seventeen, eighteen, nineteen, or twenty lift attachment sites. The number and position of the lift attachment sites associated with the fabric-like material of the lift garment is dependent upon at least the weight, shape, condition, and positioning of the subject for whom the lift garment is designed. In some embodiments, lift garment 702 includes a first reinforcing material attached to or incorporated into the at least one of the one or more lift attachment sites 708. In an aspect, the first reinforcing material is adhered or sewn to at least one surface of the fabric-like material 706 at the at least one of the one or more lift attachment sites 708. In an aspect, the first reinforcing material is woven into the fabric-like material 706 at the at least one of the one or more lift attachment sites 708.

Lift garment 702 further includes at least one lift attachment element 710 associated with the fabric-like material 706 at at least one of the one or more lift attachment sites 708. The at least one lift attachment element 710 is configured to attach the lift garment 702 to a lift apparatus 722. In an aspect, the lift attachment element 710 is glued, punched, stapled, pinned, or sewn to the fabric-like material. In an aspect, the lift attachment element 710 is an extension of the fabric-like material. In an aspect, the at least one lift attachment element 710 of the lift garment 702 includes at least one of a hook, a loop of material, or a magnet. Non-limiting aspects of lift attachment elements are described above herein.

Lift garment 702 further includes a load sensor 712 configured to measure a load. Load sensor 712 is associated with at least one of the one or more lift attachment sites 708 or along a load path between the one or more lift attachment sites 708. In some embodiments, the lift garment 702 includes two or more load sensors 712 distributed along a length of the at least one load path between the one or more lift attachment sites 708. In an aspect, the load sensor 712 of the lift garment 702 includes a force transducer. In an aspect, the load sensor 712 of the lift garment 702 includes a strain sensor, a stretch sensor, or a pressure sensor. In some embodiments, load sensor 712 is woven, knitted, laminated, printed or stitched into or onto the fabric-like material 706. Non-limiting aspects of load sensors are described above herein.

In some embodiments, lift garment 702 includes a second reinforcing material attached to or incorporated into the at least one load path between the one or more lift attachment sites 708. In an aspect, the second reinforcing material is identical to the first reinforcing material. In an aspect, the second reinforcing material is different from the first reinforcing material. In some embodiments, the second reinforcing material extends along a length of the at least one load path. In an aspect, the second reinforcing material is adhered to or sewn to at least one surface of the fabric-like material 706 along the length of the at least one load path between the one or more lift attachment sites 708. In an aspect, the second reinforcing material is woven into the fabric-like material 706 along the length of the at least one load path between the one or more lift attachment sites 708.

Returning to FIG. 7, lift garment 702 further includes microcontroller 714 including circuitry configured to receive and process information regarding the measured load from load sensor 712. Lift garment 702 further includes reporting device 716 operably coupled to microcontroller 714 and configured to transmit one or more signals indicative of the processed information regarding the measured load. In an aspect, reporting device 716 of lift garment 702 includes at least one of an optical reporting device, an audio reporting device, a haptic reporting device, or a display. In an aspect, reporting device 716 of lift garment 702 includes a transmission unit including an antenna. In some embodiments, reporting device 716 of lift garment 702 is configured to communicate with the lift control mechanism 704. In some embodiments, reporting device 716 of lift garment 702 is configured to communicate with an external device. For example, a transmission unit associated with the lift garment can be configured to wirelessly communicate with a mobile communication device or a computing device. In some embodiments, reporting device 716 of lift garment 702 is configured to communicate with an external network. For example, a transmission unit associated with the lift garment can be configured to wirelessly communicate with a health provider network. Non-limiting aspects of reporting devices have been described above herein.

Returning to FIG. 7, system 700 further includes lift control mechanism 704. Lift control mechanism 704 includes receiver 718 configured to receive the one or more signals from reporting device 716 indicative of the processed information regarding the measured load. Lift control mechanism 704 further includes circuitry 720 configured to control a function of the lift apparatus 722 in response to the one or more signals received from the reporting device 716. In an aspect, the lift control mechanism 704 is configured to control at least one of an on/off function, an up/down function, a speed function, or an acceleration function of the lift apparatus 722.

In some embodiments, microcontroller 714 of lift garment 702 includes circuitry configured to receive and process the information regarding the measured load, determine whether the measured load falls within a range of acceptable load values, and transmit a locking signal to the lift control mechanism 704 to lock the function of the lift apparatus 722 if the measured load fails to fall within the range of acceptable load values. In an aspect, the range of acceptable load values is stored in the microcontroller 714 of the lift garment 702.

In an aspect, reporting device 716 operably coupled to microcontroller 714 is configured to transmit one or more control signals to the lift control mechanism 704 based on the processed information regarding the measured load. In an aspect, reporting device 716 operably coupled to microcontroller 714 is configured to transmit at least one of an on/off signal, an up/down signal, a speed signal, or an acceleration signal to the lift control mechanism 704 based on the processed information regarding the measured load.

In some embodiments, lift garment 702 of system 700 further includes at least one load limit label including a numerical load limit associated with at least one of the one or more lift attachment sites or the at least one load path between the one or more lift attachment sites. In an aspect, the load limit label includes at least one of a color-coded label, a text-based label, or a radiofrequency tag. In an aspect, the load limit label includes a display. In some embodiments, the microcontroller of the lift garment includes circuitry configured to receive and process the information regarding the measured load, determine whether the measured load exceeds the load limit for a given lift attachment site or load path, and transmits a locking signal to the lift control mechanism to lock the function of the lift apparatus if the measure load exceeds the load limit for the given lift attachment site or load path. In an aspect, the load limits for any given lift attachment site or load path is stored in the microcontroller of the lift garment.

In some embodiments, system 700 includes one or more physiological sensors incorporated into the lift garment 702 and configured to measure at least one physiological parameter of the subject. In an aspect, at least one of the one or more physiological sensors includes a heart rate sensor, a blood pressure sensor, a respiration sensor, a temperature sensor, or a biochemical sensor. Non-limiting aspects of physiological sensors for use with a lift garment are described above herein. In some embodiments, the one or more physiological sensors are attached to at least one surface of the fabric-like material 706 of lift garment 702. In some embodiments, the one or more physiological sensors are incorporated into at least one surface of the fabric-like material 706 of lift garment 702. In some embodiments, the one or more physiological sensors are woven, knit, laminated, printed, or stitched into or onto the fabric-like material 706 of lift garment 702.

In some embodiments, the microcontroller 714 of lift garment 702 includes circuitry configured to receive and process information regarding the measured at least one physiological parameter of the subject; determine whether the measured at least one physiological parameter of the subject falls within a range of acceptable physiological parameter values; and transmit a control signal to the lift control mechanism to control a function of the lift apparatus is the measured at least one physiological parameter of the subject fails to fall within the range of acceptable physiological parameter values.

In some embodiments, the microcontroller 714 of lift garment 702 includes circuitry configured to receive and process information regarding the measured at least one physiological parameter of the subject, and recording device 716 is configured to transmit one or more signals indicative of the processed information regarding the measured at least one physiological parameter of the subject. In some embodiments reporting device 716 is configured to transmit one or more signals indicative of the processed information regarding the measured at least one physiological parameter of the subject to an external source, e.g., a mobile communication device or a computing device. In some embodiments, reporting device 716 is configured to transmit one or more signals indicative of the processed information regarding the measured at least one physiological parameter of the subject to an external network, e.g., a health provider network.

In some embodiments, at least one of the one or more physiological sensors includes at least one blood oxygenation sensor incorporated into lift garment 702. The at least one blood oxygenation sensor is configured to measure an oxygen saturation level of the subject. In an aspect, the at least one blood oxygenation sensor is incorporated into a load path between one or more lift attachment sites 708. In an aspect, the at least one blood oxygenation sensor includes a near infrared optical blood oxygenation sensor. In an aspect, the at least one blood oxygenation sensor is associated with a surface of the fabric-like material 706 configured for placement in contact with an external surface of the subject. In some embodiments, the microcontroller 714 of lift garment 706 includes circuitry configured to received and process information regarding the measured oxygen saturation level of the subject, and recording device 716 is configured to transmit one or more signals indicative of the processed information regarding the measured oxygen saturation level of the subject. For example, the recording device can transmit one or more signals indicative of the processed information regarding the measured oxygen saturation level of the subject to an external device, e.g., a mobile communication device or a computing device. For example, the recording device can transmit one or more signals indicative of the processed information regarding the measured oxygen saturation level of the subject to an external network, e.g., a health provider network.

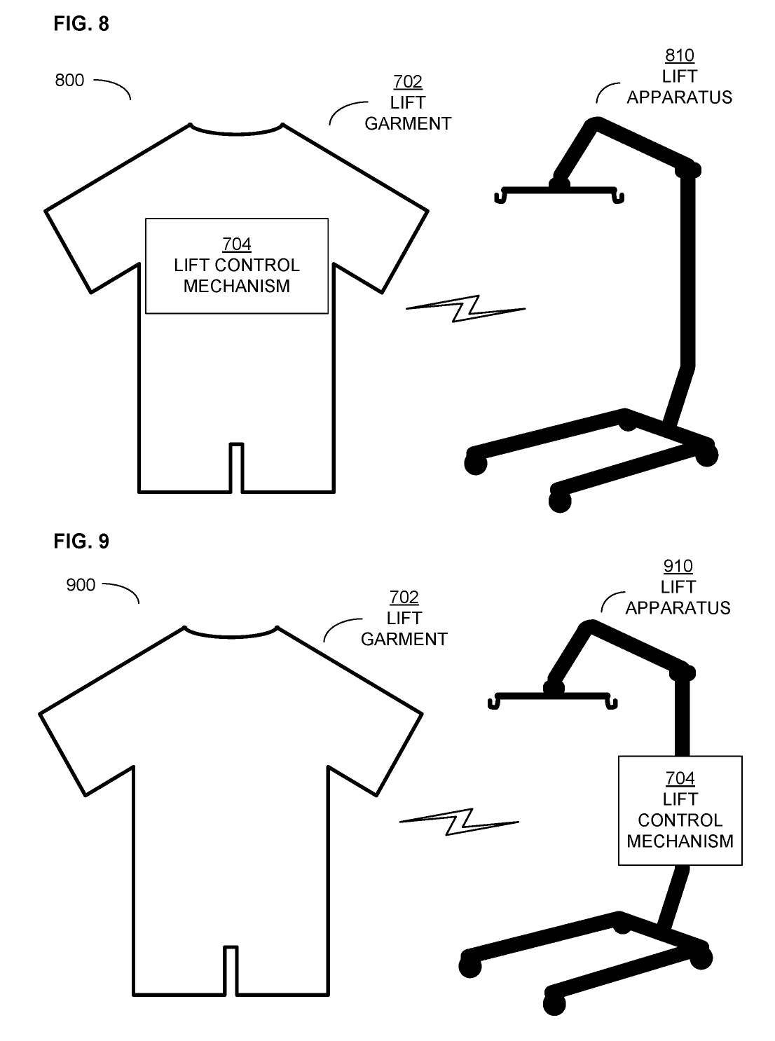

FIGS. 8 and 9 illustrate further aspects of a system. FIGS. 8 and 9 show system 800 and system 900, respectively, each including lift garment 702 and lift control mechanism 704. Lift control mechanism 704 includes a receiver configured to receive the one or more signals from the reporting device indicative of the processed information regarding the measured load and circuitry configured to control a function of a lift apparatus in response to the one or more signals received from the lift garment 702. The function can include at least one of an on/off function, an up/down function, a speed function, and/or an acceleration function of the lift apparatus. In some embodiments, as exemplified with system 800 in FIG. 8, the lift control mechanism 704 is associated with the lift garment 702 and configured to wirelessly communicate with lift apparatus 810. As such, the circuitry of the lift control mechanism is configured to wirelessly transmit a signal to control a function of lift apparatus 810. In this non-limiting embodiment, lift apparatus 810 includes a receiver capable of receiving the wirelessly transmitted control signal. For example, the lift control mechanism 704 associated with lift garment 702 can transmit a Bluetooth or similar signal type to a receiver associated with lift apparatus 810.

In some embodiments, as exemplified with system 900 in FIG. 9, the lift control mechanism 704 is associated with the lift apparatus 910 and configured to wirelessly communicate with lift garment 702. As such, the receiver of lift control mechanism 704 is configured to wirelessly receive the one or more signals from the reporting device of the lift garment indicative of the processed information regarding the measured load, and circuitry configured control a function of the lift apparatus 910 in response to the one or more signal received from reporting device of the lift garment 702. The circuitry can be configured to transmit a control signal through either a wired or wireless communication link.

Described herein are methods implemented with a lift garment. In some embodiments, a method implemented with a lift garment includes measuring a load value with a load sensor associated with the lift garment worn by a subject and attached to a lift apparatus, the lift garment a fabric-like material shaped to substantially completely encircle a torso and at least a portion of arms and legs of the subject, the load sensor, at least one lift attachment element associated with the fabric-like material at at least one of one or more lift attachment sites, a microcontroller including circuitry and a stored range of acceptable load values, and a reporting device operably coupled to the microcontroller; receiving and processing the measured load value with the circuitry of the microcontroller; determining whether the measured load value falls within the stored range of acceptable load values; and transmitting one or more signals from the reporting device indicative of whether the measured load value falls within the stored range of acceptable load values.

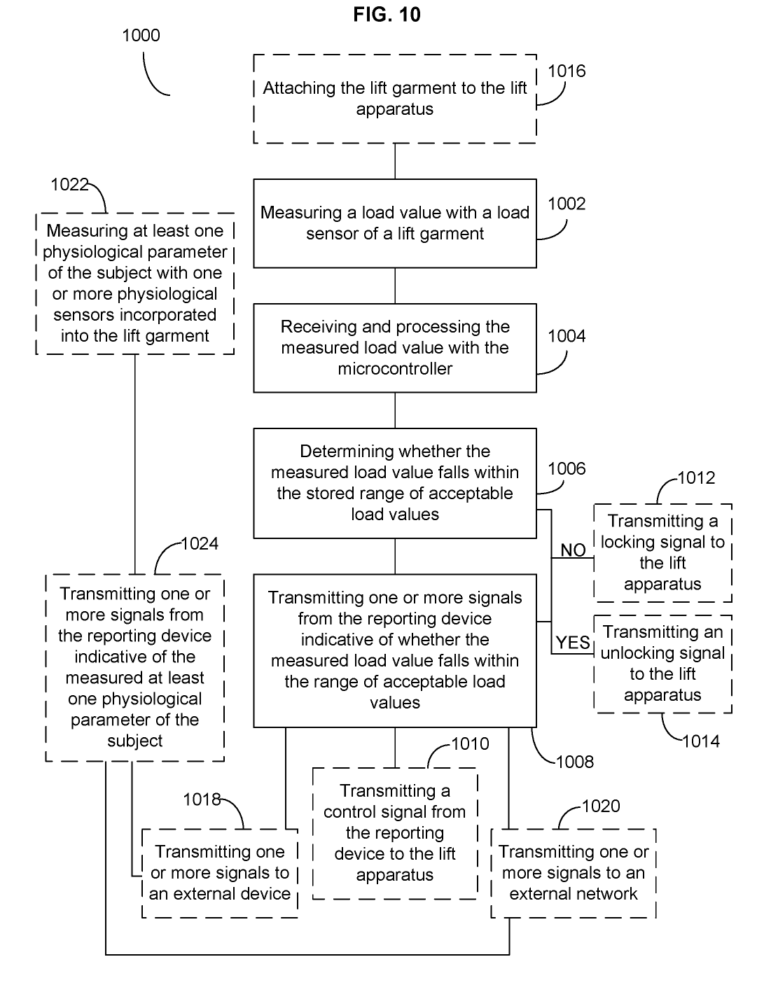

FIG. 10 shows a flow diagram illustrating aspects of method 1000 for using a lift garment. The lift garment of method 1000 includes a load sensor, one or more lift attachment sites, at least one lift attachment element, a microcontroller including circuitry, and a reporting device. Method 1000 include step 1002 of measuring a load value with a load sensor of a lift garment. In an aspect, method 1000 includes measuring the load value with at least one of a strain sensor, a stretch sensor, or a pressure sensor. In an aspect, method 1000 includes measuring the load value with a force transducer. In an aspect, method 1000 includes measuring the load value with a load sensor associated with at least one of the one or more lift attachment sites. In an aspect, method 1000 includes measuring the load value with a load sensor associated with the at least one load path between the one or more lift attachment sites. In an aspect, method 1000 includes measuring one or more load values with two or more load sensors distributed along a length of the at least one load path between the one or more lift attachment sites.

Method 1000 includes step 1004 of receiving and processing the measured load value with the microcontroller and step 1006 of determining whether the measured load value falls within the stored range of acceptable load values. For example, the method can include receiving and processing the measured load value from the load sensor with the microcontroller associated with the lift garment. For example, the method can include determining whether the measured load value exceeds an acceptable load value. In an aspect, the range of acceptable load values is stored in a memory component of the microcontroller.

Method 1000 further includes step 1008 of transmitting one or more signals from the reporting device indicative of whether the measured load value falls within the range of acceptable load values. In an aspect, method 1000 includes transmitting at least one of an optical signal, an audio signal, or a haptic signal from the reporting device indicative of whether the measured load value falls within the stored range of acceptable load values. In an aspect, the method 1000 includes transmitting one or more light signals with one or more color-coded lights associated with the lift garment. In an aspect, method 1000 includes transmitting one or more audio signals through speakers associated with the lift garment. In an aspect, method 1000 includes transmitting one or more haptic signals through a haptic reporting device associated with the lift garment. In an aspect, method 1000 includes transmitting one of more signals through a transmission unit including an antenna.

In some embodiments, method 1000 includes step 1010 of transmitting a control signal from the reporting device to the lift apparatus. In an aspect, method 1000 includes transmitting a control signal from the reporting device to the lift apparatus to control at least one or an on/off function, an up/down function, a speed function, or an acceleration function of the lift apparatus is response to the measured load value. For example, if the measured load value is close to a load limit value, the reporting device may transmit a control signal to decrease the speed of lifting. For example, if the measured load value is outside the stored range of acceptable load values or is off balance, the reporting device may transmit an off signal to stop the lift apparatus from functioning. In an aspect, the reporting device wirelessly transmits the control signal to the lift apparatus in response to the processed information regarding the measured load value.

In some embodiments, method 1000 can include transmitting a signal from the lift garment to control a function of a lift apparatus based on a “yes” or “no” determination from step 1006 of determining whether the measured load value falls within a range of acceptable load values. In an aspect, method 1000 includes step 1012 of transmitting a locking signal to the lift apparatus. In an aspect, method 1000 includes transmitting a locking signal from the reporting to the lift apparatus if the measured load value fails to fall within the range of acceptable load values. In an aspect, method 1000 includes step 1014 of transmitting an unlocking signal to the lift apparatus. In an aspect, method 1000 includes transmitting an unlocking signal from the reporting device to the lift apparatus if the measured load value falls within the range of acceptable load values. In an aspect, method 1000 includes wirelessly transmitting a locking or an unlocking signal to the lift apparatus. In some embodiments, method 1000 further includes comparing a measured load value from a first load sensor and a measured load value from a second load sensor and transmitting an unlocking signal if the difference between the measured load value from the first load sensor and the measured load value from the second load sensor falls within a range of acceptable differential load values.

In some embodiments, method 1000 includes step 1016 of attaching the lift garment to the lift apparatus. In an aspect, method 1000 includes attaching the lift garment to the lift apparatus with the at least one lift attachment element associated with the fabric-like material. In an aspect, method 1000 includes attaching the lift garment to the lift apparatus with at least one of a hook, a loop of material, or a magnet associated with the fabric-like material.

In some embodiments, method 1000 includes step 1018 of transmitting one or more signals to an external device. In an aspect, method 1000 includes transmitting the one or more signals from the reporting device to an external device. For example, the method can include transmitting one or more signals to an external device from an antenna of a transmission unit associated with the lift garment. In some embodiments, method 1000 includes transmitting one or more signals from the reporting device to a mobile communication device. For example, the method can include transmitting one or more signals to a smart phone. In some embodiments, method 1000 includes transmitting one or more signals from the reporting device to a computing device. For example, the method can include transmitting one or more signals to a tablet, laptop, or desktop computing device. In an aspect, method 1000 includes transmitting the one or more signals to an external device located in the same room with the lift garment worn by the subject. For example, the method can include transmitting the one or more signals to a mobile communication device or a computing device located in a hospital, medical clinic, skilled nursing, or assisted living facility in which the subject is located. For example, the method can include transmitting the one or more signals to a mobile communication device or a computing device located in a residence in which the subject is located. In an aspect, method 1000 includes transmitting the one or more signals to an external device located in a remote location relative to the location lift garment worn by the subject. For example, the method can include transmitting the one or more signals to a remote mobile communication device or a computing device associated with a physician or other healthcare provider.

In some embodiments, method 1000 includes step 1020 of transmitting one or more signals to an external network. In an aspect, method 1000 includes transmitting the one or more signals from the reporting device to an external network. In an aspect, method 1000 includes transmitting the one or more signals from the reporting device to a health provider network. For example, the method can include wirelessly transmitting the one or more signals from an antenna of a transmission unit associated with the lift garment to a network associated with a hospital, medical clinic, skilled nursing facility, or assisted living facility. For example, the method can include wirelessly transmitting the one or more signals to the subject's medical record stored in a health provider network.

In some embodiments, method 1000 further includes step 1022 of measuring at least one physiological parameter of the subject with one or more physiological sensors incorporated into the lift garment. In an aspect, method 1000 includes measuring the at least one physiological parameter of the subject with at least one of a heart beat sensor, a blood pressure sensor, a temperature sensor, a respiration sensor, or a biochemical sensor incorporated into the lift garment. For example, the method can include measuring at least one of heart beat, blood pressure, respiration, temperature, of biochemical parameter of the subject while wearing the lift garment. For example, the method can include measuring at least one physiological parameter of the subject before, during, and/or after a transfer procedure using the lift garment and a lift apparatus. For example, the method can include measuring at least one physiological parameter of the subject prior to being attached to the lift apparatus. For example, the method can include measuring at least one physiological parameter of the subject while the subject is suspended from the lift apparatus. For example, the method can include measuring at least one physiological parameter of the subject after the subject has been released from the lift apparatus. Method 1000 further includes step 1024 of transmitting one or more signals from the reporting device indicative of the measured at least one physiological parameter of the subject. In an aspect, method 1000 includes transmitting at least one of an optical signal, an audio signal, or a haptic signal from the reporting device indicative of the measured at least one physiological parameter of the subject. In an aspect, method 1000 includes transmitting one or more signals indicative of the measured at least one physiological parameter of the subject to an external device, e.g., a mobile communication device or a computing device. In an aspect, method 1000 includes transmitting one or more signals indicative of the measured at least one physiological parameter of the subject to an external network, e.g., a health provider network. For example, the method can include transmitting one or more signals indicative of a measured physiological parameter, e.g., blood pressure or blood oxygenation, directly into the subject's electronic medical file associated with a health provider network.

In some embodiments, method 1000 includes receiving and processing information with the microcontroller regarding the measured at least one physiological parameter of the subject; determining whether the measured at least one physiological parameter of the subject falls within a range of acceptable physiological parameter values; and transmitting a control signal to the lift apparatus to control operation of the lift apparatus if the measured at least one physiological parameter of the subject fails to fall within the range of acceptable physiological parameter values.

In some embodiments, method 1000 further includes measuring an oxygen saturation level of the subject with one or more blood oxygenation sensors associated with the lift garment; receiving and processing information with the microcontroller regarding the measured oxygen saturation level of the subject; determining whether the measured oxygen saturation level of the subject falls within a range of acceptable oxygen saturation levels; and transmitting a control signal to the lift apparatus to control operation of the lift apparatus based on whether the measured oxygen saturation level of the subject falls within the range of acceptable oxygen saturation levels.

Described herein are aspects of a lift garment including one or more physiological sensors. In some embodiments, a lift garment includes a fabric-like material shaped to substantially completely encircle a torso and at least a portion of arms and legs of a subject, the fabric-like material including one or more lift attachment sites; at least one lift attachment element associated with the fabric-like material at at least one of the one or more lift attachment sites, the at least one lift attachment element configured to attach the lift garment to a lift apparatus; one or more physiological sensors configured to measure at least one physiological parameter of the subject; a microcontroller including circuitry configured to receive and process information regarding the measured at least one physiological parameter of the subject; and a reporting device operably coupled to the microcontroller and configured to transmit one or more signals indicative of the processed information regarding the measured at least one physiological parameter of the subject.

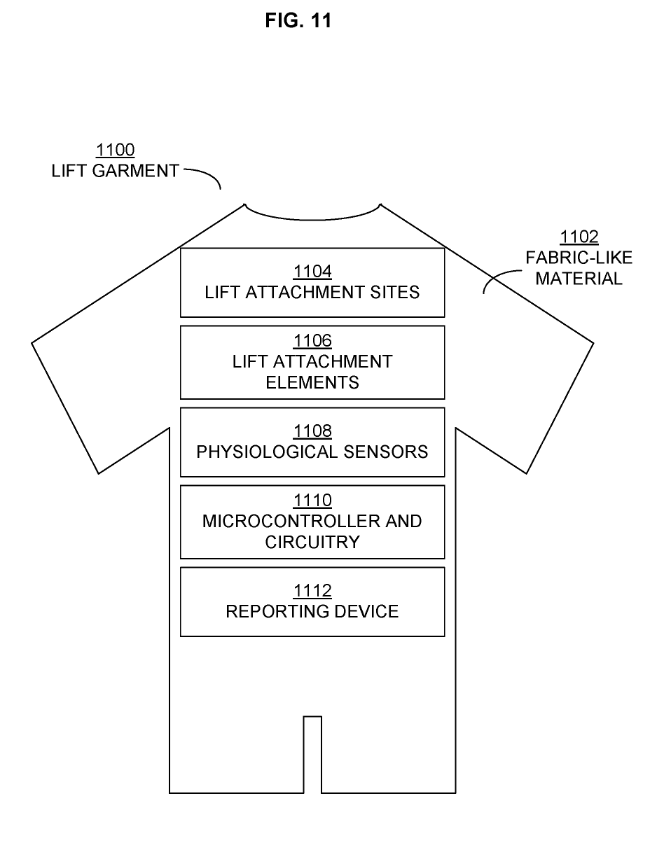

FIG. 11 illustrates aspects of a lift garment including one or more physiological sensors. Lift garment 1100 includes a fabric-like material 1102 shaped to substantially completely encircle a torso and a least a portion of arms and legs of a subject. In an aspect, the fabric-like material 1102 is shaped to substantially completely encircle the torso and at least a portion of the arms and legs of a human subject. For example, the fabric-like material can be shaped as a piece of clothing, e.g., a jumpsuit or “onesie.” In an aspect, at least a portion of the fabric-like material 1102 of the lift garment 1100 includes a woven material or a knit material. In an aspect, at least a portion of the fabric-like material 1102 of the lift garment 1100 includes a non-woven material. In an aspect, at least a portion of the fabric-like material 1102 of the lift garment 1100 is formed from at least one polymer type. Non-limiting aspects of fabric-like materials are described above herein.

Fabric-like material 1102 of lift garment 1100 includes one or more lift attachment sites 1104. In an aspect, at least one of the one or more lift attachment sites 1104 is associated with a front portion of the fabric-like material 1102. For example, a lift garment designed for a human subject might include one or more lift attachment sites situated near a front portion of the shoulders, arms pits, hips and/or thighs of the human subject. In an aspect, at least one of the one or more lift attachment sites 1104 is associated with a back portion of the fabric-like material 1102. For example, a lift garment designed for lifting a large animal subject, e.g., a horse, might include one or more lift attachment sites associated with a portion of the lift garment position on the back of the animal.

In some embodiments, the lift garment 1100 further includes a first reinforcing material attached to or incorporated into at least one of the one or more lift attachment sites. In an aspect, the first reinforcing material is adhered or sewn to at least one surface of the fabric-like material at the at least one of the one or more lift attachment sites. In an aspect, the first reinforcing material is woven into the fabric-like material at the at least one of the one or more lift attachment sites.

In some embodiments, the lift garment 1100 includes a second reinforcing material attached to or incorporated into at least one load path between the one or more lift attachment sites, wherein the second reinforcing material extends along at least a portion of the at least one load path. In an aspect, the second reinforcing material extends along the at least a portion of the at least one load path associated with a back portion of the fabric-like material. In an aspect, the second reinforcing material is adhered or sewn to at least one surface of the fabric-like material along the at least a portion of the at least one load path. In an aspect, the second reinforcing material is woven into the fabric-like material along the at least a portion of the at least one load path.

In some embodiments, the lift garment 1100 includes a structural platform associated with the fabric-like material 1102. FIG. 3C illustrates a non-limiting example of a structural platform associated with the fabric-like material of a lift garment. In an aspect, the structural platform provides structural support to the subject during a lift activity. In an aspect, the lift garment includes a single structural platform. In an aspect, the structural platform is formed from a fabric-like material. In an aspect, the structural platform is formed from plastic. In an aspect, the structural platform is formed from metal. In an aspect, the structural platform is attached to a surface of the fabric-like material. In an aspect, the structural platform is insertable into a pocket or pouch associated with the lift garment. In an aspect, the structural platform spans a space between two or more edges of the fabric-like material. In an aspect, the structural platform brings the two or more edges of the fabric-like material together to substantially completely encircle the torso and at least a portion of the arms and legs of the subject. In an aspect, the structural platform includes one or more of the one or more physiological sensors, the microcontroller with the circuitry, or the reporting device. For example, the structural platform can include an integrated circuit board including one or more of a heart rate sensor, a blood pressure sensor, a respiration sensor, a temperature sensor, and/or a biochemical sensor. For example, the structural platform can include an integrated circuit board including one or more blood oxygenation sensors. In an aspect, the structural platform includes a circuit board including sensors, a microcontroller, circuitry and a reporting device. In an aspect, the structural platform includes at least one load sensor.

In some embodiments, lift garment 1100 is part of a system that includes lift garment 1100 and a removable structural platform, the lift garment including a means for securing the structural platform to the fabric-like material of the lift garment. The means for securing the structural platform can include one or more of a pocket, a pouch, a hook and loop fastener, snaps, an adhesive, straps, and the like for securing the structural platform to the lift garment. In an aspect, the structural platform is removable from the lift garment. For example, the structural platform can be removed to allow for washing/sanitizing of the lift garment. In an aspect, the system further includes sensors, a microcontroller with circuitry, and a reporting device associated with the removable structural platform. In an aspect, the system further includes one or more physiological sensors associated with the removable structural platform. In an aspect, the system further includes at least one blood oxygenation sensor associated with the removable structural platform.

Lift garment 1100 further includes at least one lift attachment element 1106 associated with the fabric-like material 1102 at at least one of the one or more lift attachment sites 1104. The at least one lift attachment element 1106 is configured to attach the lift garment 1100 to a lift apparatus. Lift garment 1100 further includes one or more physiological sensors 1108 configured to measure at least one physiological parameter of the subject. Lift garment 1100 further includes a microcontroller and circuitry 1110 configured to receive and process information regarding the measured at least one physiological parameter of the subject and a reporting device 1112 operably coupled to the microcontroller 1110 and configured to transmit one or more signals indicative of the processed information regarding the measured at least one physiological parameter of the subject. In an aspect, microcontroller 1110 includes circuitry configured to receive and process the information regarding the measured at least one physiological parameter of the subject and to determine whether the measured at least one physiological parameter of the subject falls within a range of acceptable physiological parameter values; and wherein reporting device 1112 is configured to transmit a control signal to the lift apparatus to control operation of the lift apparatus if the measured at least one physiological parameter of the subject fails to fall within the range of acceptable physiological parameter values.

In an aspect, microcontroller 1110 can include a microprocessor, a central processing unit (CPU), a digital signal processor (DSP), application-specific integrated circuit (ASIC), a field programmable gate entry (FPGA), or the like, or any combinations thereof, and can include discrete digital or analog circuit elements or electronics, or combinations thereof. The microcontroller can further include signal processing algorithms, e.g., band pass filters, low pass filters, or any other single processing algorithms or combinations thereof. Non-limiting aspects of a microcontroller are described above herein.

In an aspect, the microcontroller 1110 includes circuitry configured to receive and process information regarding measurement at a first time point of the at least one physiological parameter of the subject; receive and process information regarding measurement at a second time point of the at least one physiological parameter of the subject; and determine if the rate of change between the measurement at the first time point and the measurement at the second time point falls within a range of acceptable rate changes; and wherein the reporting device 1112 is configured to transmit a control signal to the lift apparatus to control operation of the lift apparatus if the rate of change between the measurement at the first time point and the measurement at the second time point fails to fall within the range of acceptable rate changes.

In some embodiments, microcontroller 1110 includes circuitry configured to receive and process information regarding measurement at a first time point of the at least one physiological parameter of the subject; receive and process information regarding measurement at a second time point of the at least one physiological parameter of the subject; and determine if a rate of change between the measurement at the first time point and the measurement at the second time point falls within a range of acceptable rate changes; and wherein the reporting device 1112 is configured to transmit a control signal to the lift apparatus to control operation of the lift apparatus if the rate of change between the measurement at the first time point and the measurement at the second time point fails to fall within the range of acceptable rate changes.

In an aspect, at least one of the microcontroller 1110 including the circuitry, or the reporting device 1112 is woven, knitted, laminated, printed, or stitched into or onto the fabric-like material 1102

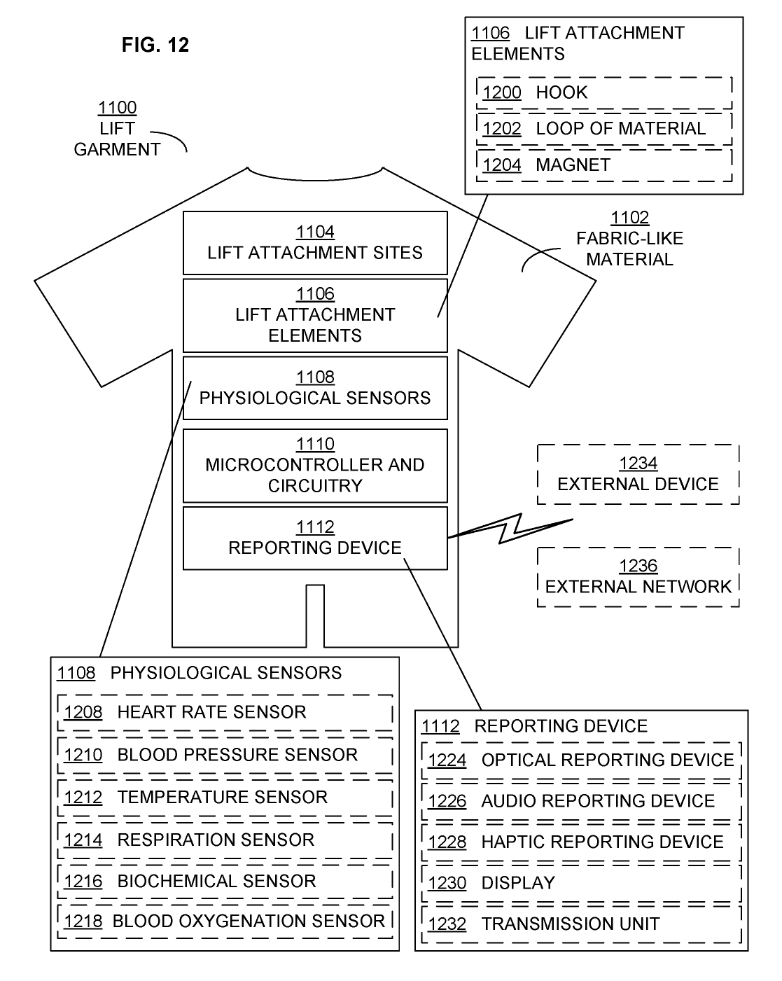

FIG. 12 illustrates further aspects of a lift garment including one or more physiological sensors. Lift garment 1100 includes at least one lift attachment element 1106 configured to attach lift garment 1100 to a lift apparatus. In an aspect, the at least one lift attachment element includes a hook 1200. For example, the lift garment can include a clip hook that attaches either directly or indirectly to a spreader bar or cradle of a lift apparatus. In an aspect, the at least one lift attachment element includes a loop of material 1202. For example, the lift garment can include a grommet-reinforced opening defined by the fabric-like material of the lift garment. In an aspect, the at least one lift attachment element includes a magnet 1204. For example, the lift garment can include one or more magnets sewn into the fabric-like material of the lift garment at the one or more lift attachment sites. Non-limiting aspects of lift attachment elements have been described above herein.

Lift garment 1100 further includes one or more physiological sensors 1108 configured to measure at least one physiological parameter of the subject. In an aspect, at least one of the one or more physiological sensors 1108 includes a heart rate sensor 1208. In an aspect, at least one of the one or more physiological sensors 1108 includes a blood pressure sensor 1210. In an aspect, at least one of the one or more physiological sensors 1108 includes a temperature sensor 1212. In an aspect, at least one of the one or more physiological sensors 1108 includes a respiration sensor 1214. In an aspect, at least one of the one or more physiological sensors 1108 includes a biochemical sensor 1216. Non-limiting aspects of physiological sensors have been described above herein.

In an aspect, at least one of the one or more physiological sensors 1108 includes at least one blood oxygenation sensor 1218. In an aspect, the at least one blood oxygenation sensor 1218 is associated with the at least one load path between the one or more lift attachment sites 1104. In an aspect, the at least one blood oxygenation sensor 1218 includes a near infrared optical blood oxygenation sensor. In an aspect, the at least one blood oxygenation sensor 1218 is associated with a surface of the fabric-like material 1102 configured for placement in contact with an external surface of the subject.

In an aspect, the one or more physiological sensors 1108 are associated with at least one surface of the fabric-like material 1102. In an aspect, at least one of the one or more physiological sensors 1108 is associated with a surface of the fabric-like material 1102 configured for placement in contact with an external surface of the subject. For example, an ECG electrode can be positioned on the inner surface of the lift garment so as to contract a skin surface of the subject. In an aspect, at least one of the one or more physiological sensors 1108 is attached to at least one surface of the fabric-like material 1102. For example, the one or more physiological sensors can be sewn, glued, or otherwise attached to at least one surface of the fabric-like material of the lift garment. In an aspect, at least one of the one or more physiological sensors 1108 is incorporated into at least one surface of the fabric-like material 1102. In an aspect, at least one of the one or more physiological sensors 1108 is woven, knitted, laminated, printed, or stitched into or onto the fabric-like material 1102.

Lift garment 1100 further includes reporting device 1112 operably coupled to the microcontroller 1110 and configured to transmit one or more signals indicative of the processed information regarding the measured at least one physiological parameter of the subject. In an aspect, the one or more signals can include at least one of an optical signal, an audible signal, or a haptic signal. In an aspect, the reporting device 1112 includes an optical reporting device 1224. For example, the reporting device can include one or more color-coded lights for transmitting an optical signal indicative of the measured at least one physiological parameter of the subject. In an aspect, the reporting device 1112 includes an audio reporting device 1226 including at least one speaker. For example, the reporting device can include a sound card with a small speaker for transmitting an audible signal indicative of the measured at least one physiological parameter of the subject. In an aspect, the reporting device 1112 includes a haptic reporting device 1228. For example, the reporting device can include a vibration generator for transmitting a haptic signal indicative of the measured at least one physiological parameter of the subject. In an aspect, the reporting device 1112 includes a display 1230. In an aspect, the reporting device 1112 includes a transmission unit 1232. Non-limiting aspects of reporting devices have been described above herein.

In some embodiments, reporting device 1112 is configured to transmit a control signal to the lift apparatus to control operation of the lift apparatus if a measured at least one physiological parameter fails to fall within a range of acceptable physiological parameter values. For example, the lift garment can include a transmission unit operably coupled to the microcontroller and configured to wirelessly transmit a control signal to the lift apparatus to control an operation of the lift apparatus, the type of operation dependent upon the measured at least one physiological parameter of the subject. In an aspect, reporting device 1112 is configured to transmit a control signal to the lift apparatus to control at least one of an on/off function, an up/down function, an acceleration function, or a speed function of the lift apparatus. In an aspect, reporting device 1112 is configured to transmit a locking signal to the lift apparatus. In an aspect, reporting device 1112 is configured to transmit an unlocking signal to the lift apparatus.

In some embodiments, reporting device 1112 is configured to communicate with an external device 1234. In an aspect, reporting device 1112 is configured to communicate wirelessly with an external device 1234. In an aspect, reporting device 1112 is configured to communicate with the lift apparatus. In an aspect, reporting device 1112 is configured to communicate with a mobile communication device. For example, the reporting device can be configured to communicate with a smart phone that includes a program, set of instructions, and/or application configured to receive information from the reporting device, process the information, and display the information to a user. In an aspect, reporting device 1112 is configured to communicate with a computing device. For example, the reporting device can be configured to communicate with a tablet computer used by a caregiver in a hospital, skilled nursing, assisted living facility, or other healthcare environment. In an aspect, reporting device 1112 is configured to communicate with an external network 1236. In an aspect, reporting device 1112 is configured to communicate with a health provider network. In an aspect, reporting device 1112 is configured to communicate directly with a subject's electronic medical record.

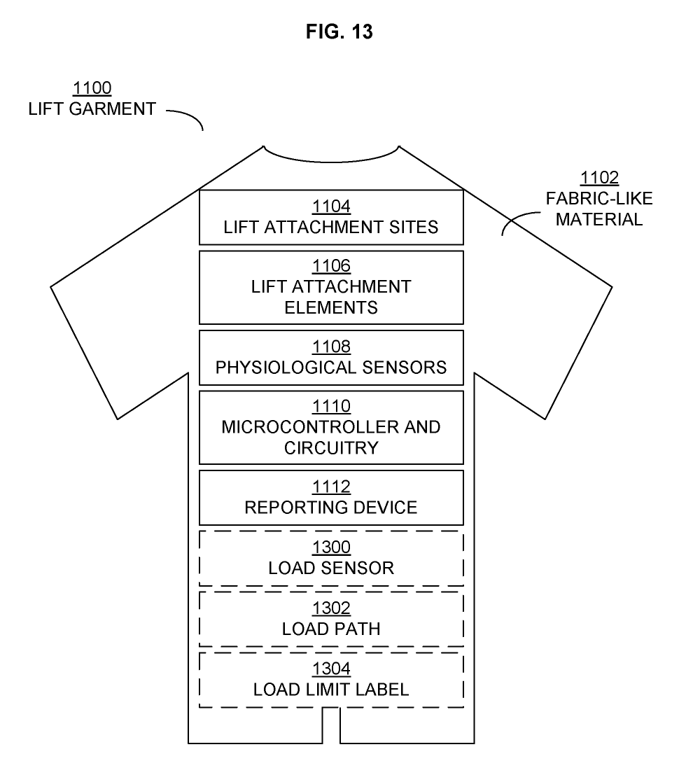

FIG. 13 illustrates further aspects of a lift garment. In some embodiments, lift garment 1100 includes at least one load sensor 1300 configured to measure a load, the at least one load sensor 1300 associated with at least one of the one or more lift attachment sites 1104 or at least one load path 1302 between the one or more lift attachment sites 1104. In an aspect, the lift garment includes a plurality of load sensors. In an aspect, the lift garment 1100 includes two or more load sensors 1300 distributed along a length of the at least one load path 1302 between the one or more lift attachment sites. In an aspect, the at least one load sensor 1300 includes a force transducer. In an aspect, the at least one load sensor 1300 includes at least one of a strain sensor, a stretch sensor, or a pressure sensor.

In an aspect, the microcontroller 1110 includes circuitry configured to receive and process information regarding the measured load and the reporting device 1112 is configured to transmit one or more signals indicative of the processed information regarding the measured load. For example, the reporting device can transmit one or more of an optical signal, an audio signal, or a haptic signal indicative of the processed information regarding the measured load. In an aspect, the microcontroller 1110 includes circuitry configured to receive and process information regarding the measured load and to determine whether the measured load falls within a range of acceptable load values; and the reporting device 1112 is configured to transmit a locking signal to the lift apparatus to block operation of the lift apparatus if the measured load fails to fall within the range of acceptable load values.

In some embodiments, lift garment 1100 further includes at least one load limit label 1304. The at least one load limit label 1304 includes at least one of a numerical load limit associated with each of the one or more lift attachment sites 1104 or a numerical load limit associated with a load path 1302 between the one or more lift attachment sites 1104. In an aspect, the at least one load limit label includes at least one of a color-coded label, a text-based label, an RFID tag, or a small display. Other aspects of load limit labels have been described above herein.

Described herein are aspects of a system including a lift garment with one or more physiological sensors and a lift control mechanism. In some embodiments, the system includes a lift garment including a fabric-like material shaped to substantially completely encircle a torso and at least a portion of arms and legs of a subject, the fabric-like material including one or more lift attachment sites, at least one lift attachment element associated with the fabric-like material at at least one of the one or more lift attachment sites, the at least one lift attachment element configured to attach the lift garment to a lift apparatus, one or more physiological sensors configured to measure at least one physiological parameter of the subject, a microcontroller including circuitry configured to receive and process information regarding the measured at least one physiological parameter of the subject, and a reporting device operably coupled to the microcontroller and configured to transmit one or more signals indicative of the processed information regarding the measured at least one physiological parameter of the subject; and a lift mechanism including a receiver configured to receive the one or more signals from the reporting device indicative of the processed information regarding the measured at least one physiological parameter of the subject, and circuitry configured to control a function of the lift apparatus in response to the one or more signals received from the reporting device.

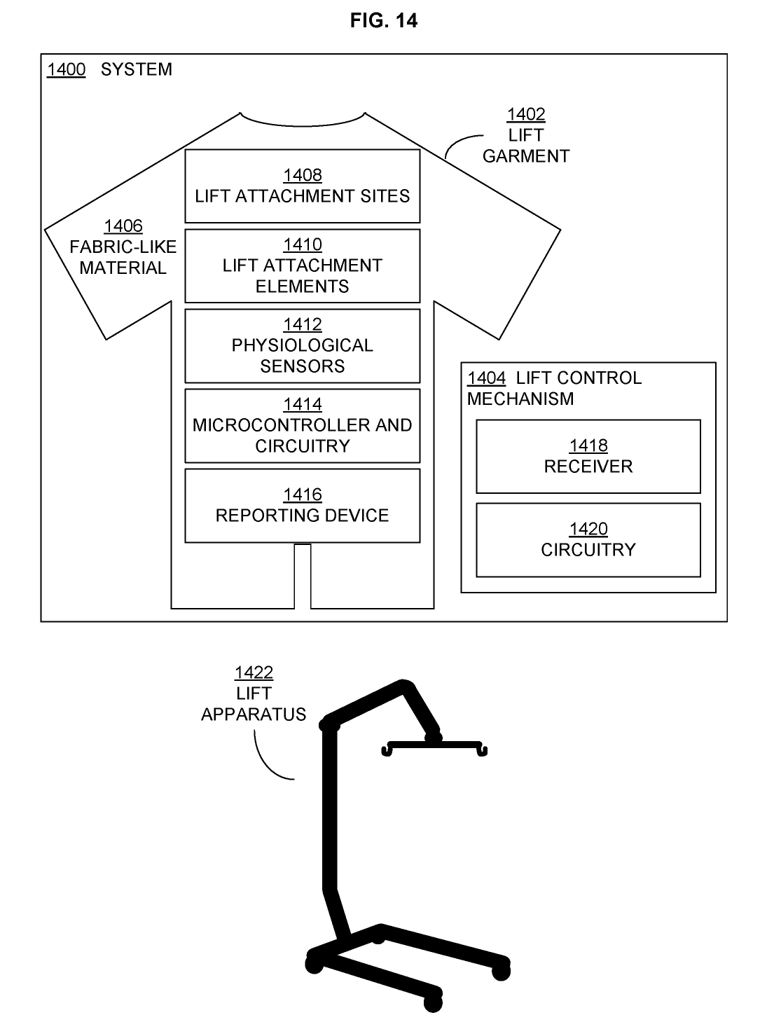

FIG. 14 illustrates aspects of a system including a lift garment with one or more physiological sensors and a lift control mechanism. System 1400 includes lift garment 1402 and lift control mechanism 1404. Lift garment 1402 includes a fabric-like material 1406 shaped to substantially completely encircle a torso and at least a portion of arms and legs of a subject. In some embodiments, the fabric-like material 1406 is shaped to substantially completely encircle the torso and at least a portion of the arms and legs of a human subject. In an aspect, at least a portion of the fabric-like material 1406 of lift garment 1402 includes woven material or a knit material. In an aspect, at least a portion of the fabric-like material 1406 of lift garment 1402 includes non-woven material. In an aspect, at least a portion of the fabric-like material 1406 of lift garment 1402 is formed from at least one polymer type. Further aspects of fabric-like material for use with a lift garment are presented above herein.

In some embodiments, the lift garment 1402 includes a first reinforcing material attached to or incorporated into at least one of the one or more lift attachment sites 1408. In some embodiments, the lift garment 1402 includes a second reinforcing material attached to or incorporated into at least one load path between the one or more lift attachment sites 1408, wherein the second reinforcing material extends along at least a portion of the at least one load path.

The fabric-like material 1406 of lift garment 1402 includes one or more lift attachment sites 1408. In an aspect, at least one of the one or more lift attachment sites 1408 is associated with a front portion of the fabric-like material 1406. In an aspect, at least one of the one or more lift attachment sites 1408 is associated with a back portion of the fabric-like material 1406. Lift garment 1402 of system 1400 further includes at least one lift attachment element 1410 associated with the fabric-like material 1406 at at least one of the one or more lift attachment sites 1408. In an aspect, the at least one lift attachment element 1410 of lift garment 1402 includes at least one of a hook, a loop of material, or a magnet.

Lift garment 1402 further includes one or more physiological sensors 1412 configured to measure at least one physiological parameter of the subject. In an aspect, at least one of the one or more physiological sensors 1412 includes at least one of a heart rate sensor, a blood pressure sensor, a temperature sensor, a respiration sensor, or a biochemical sensor. In an aspect, the one or more physiological sensors 1412 are associated with at least one surface of the fabric-like material 1406. In an aspect, at least one of the one or more physiological sensors 1412 is associated with a surface of the fabric-like material 1406 configured for placement in contact with an external surface of the subject. In an aspect, at least one of the one or more physiological sensors 1412 is attached to at least one surface of the fabric-like material 1406. In an aspect, at least one of the one or more physiological sensors 1412 is incorporated into at least one surface of the fabric-like material 1406. In an aspect, at least one of the one or more physiological sensors 1412 is woven, knitted, laminated, printed, or stitched into or onto the fabric-like material 1406.

In an aspect at least one of the one or more physiological sensors 1412 includes at least one blood oxygenation sensor. In an aspect, the at least one blood oxygenation sensor is associated with at least one load path between the one or more lift attachment sites 1408. In an aspect, the at least one blood oxygenation sensor includes a near infrared optical blood oxygenation sensor. In an aspect, the at least one blood oxygenation sensor is associated with a surface of the fabric-like material 1406 configured for placement in contact with an external surface of the subject.

Lift garment 1402 of system 1400 further includes a microcontroller 1414 including circuitry configured to receive and process information regarding the measured at least one physiological parameter of the subject. Lift garment 1402 of system 1400 further includes a reporting device 1416 operably coupled to the microcontroller 1414 and configured to transmit one or more signals indicative of the processed information regarding the measured at least one physiological parameter of the subject. In an aspect, the reporting device 1416 operably coupled to the microcontroller 1414 includes at least one of an optical reporting device, an audio reporting device, a haptic reporting device, or a display. In an aspect, the reporting device 1416 operably coupled to the microcontroller 1414 includes a transmission unit including an antenna. In an aspect, the reporting device 1416 operably coupled to the microcontroller 1414 is configured to communicate with an external device, e.g., the lift control mechanism, the lift apparatus, a mobile communication device and/or a computing device. In an aspect, the reporting device 1416 operably coupled to the microcontroller 1414 is configured to communicate with an external network, e.g., a health provider network.

System 1400 further includes lift control mechanism 1404 including a receiver 1418 configured to receive the one or more signals from the reporting device 1416 indicative of the processed information regarding the measured at least one physiological parameter of the subject. The lift control mechanism 1404 further includes circuitry 1420 configured to control a function of the lift apparatus 1422 in response to the one or more signals received from the reporting device 1416. In some embodiments, the lift control mechanism 1404 is associated with the lift garment 1402 and configured to wirelessly communicate with the lift apparatus 1422. In some embodiments, the lift control mechanism 1404 is associated with the lift apparatus 1422 and configured to wirelessly communicate with the lift garment 1402. The lift control mechanism 1404 is configured to control at least one of an on/off function, an up/down function, a speed function, or an acceleration function of the lift apparatus 1422. Reporting device 1416 operably coupled to the microcontroller 1414 is configured to transmit one or more control signals to the lift control mechanism 1404 to control at least one of an on/off function, an up-down function, a speed function, or an acceleration function of the lift apparatus 1422 in response to the measured at least one physiological parameter of the subject.

In some embodiments, the microcontroller 1414 of lift garment 1402 includes circuitry configured to receive and process the information regarding the measured at least one physiological parameter of the subject, and determine whether the measured at least one physiological parameter of the subject falls with a range of acceptable physiological parameter values; and wherein the reporting device 1416 operably coupled to the microcontroller 1414 is configured to transmit one or more control signals to the lift control mechanism 1404 to change operation of the lift apparatus 1422 if the measured at least one physiological parameter fails to fall within the range of acceptable physiological parameter values. The reporting device 1416 operably coupled to the microcontroller 1414 is configured to transmit a control signal to at least one of block operation of the lift apparatus 1422, at least partially reverse operation of the lift apparatus 1422, slow operation of the lift apparatus 1422, or accelerate operation of the lift apparatus 1422. In an aspect, the range of acceptable physiological parameter values is stored in the microcontroller 1414 of the lift garment 1402.

In an aspect, microcontroller 1414 includes circuitry configured to receive and process information regarding measurement of the at least one physiological parameter of the subject at a first time point; receive and process information regarding measurement of the at least one physiological parameter of the subject at a second time point; and determine if a rate of change between the measurement at the first time point and the measurement at the second time point falls within a range of acceptable rate changes; and wherein the reporting device 1416 operably coupled to the microcontroller 1414 is configured to transmit a control signal to the lift control mechanism 1404 to control operation of the lift apparatus 1422 if the rate of change between the measurement at the first time point and the measurement at the second time point fails to fall within the range of acceptable rate changes. In an aspect, the range of acceptable rate changes is stored in the microcontroller 1414.

In some embodiments, lift garment 1402 of system 1400 includes one or more physiological sensors 1412 configured to measure at least one physiological parameter of the subject predictive of hypoxia. For example, lift garment can include one or more of a heart rate sensor, a blood pressure sensor, a respiration sensor, or a blood oxygenation sensor. In an aspect, the microcontroller 1414 of the lift garment 1402 includes circuitry configured to receive and process information regarding the measured at least one physiological parameter of the subject indicative of hypoxia, and determine whether the measured at least one physiological parameter of the subject indicative of hypoxia indicates that the subject is experiencing hypoxia; and wherein the reporting device 1416 operably coupled to the microcontroller 1414 is configured to transmit one or more control signals to the lift control mechanism 1404 to change operation of the lift apparatus 1422 if the subject is experiencing hypoxia. For example, if the microcontroller of the lift garment determines that the oxygen saturation of the subject is below normal (e.g., below 90%), the reporting device transmits a control signal to the lift control mechanism to change operation of the lift apparatus.

In some embodiments, the lift garment 1402 of system 1400 includes a load sensor configured to measure a load, the load sensor associated with at least one of the one or more lift attachment sites 1408 or at least one load path between the one or more lift attachment sites 1408. In an aspect, the load sensor includes a force transducer. In an aspect, the load sensor includes a strain sensor, a stretch sensor, or a pressure sensor. In an aspect, microcontroller 1414 includes circuitry configured to receive and process information regarding the measured load and reporting device 1416 is configured to transmit one or more signals indicative of the processed information regarding the measured load. For example, the reporting device can transmit a signal to the lift control mechanism if the measured load fails to fall within a range of acceptable load values.

In some embodiments, the lift garment 1402 of system 1400 includes at least one load limit label including a numerical load limit associated with at least one of the one or more lift attachments sites 1408 or the at least one load path between the one or more lift attachment sites 1408. The load limit label includes at least one of a color-coded label, a text-based label, a radiofrequency identification (RFID) tag, or a display.

Described herein are aspects of a method implemented with a lift garment including, but not limited to, measuring at least one physiological parameter of a subject with one or more physiological sensors associated with the lift garment worn by a subject, the lift garment including a fabric-like material shaped to substantially completely encircle a torso and at least a portion of arms and legs of the subject; the one or more physiological sensors; at least one lift attachment element associated with the fabric-like material at at least one of one or more lift attachment sites; a microcontroller including circuitry and a stored range of acceptable physiological parameter values; and a reporting device operably coupled to the microcontroller; receiving and processing information regarding the measured at least one physiological parameter of the subject with the circuitry of the microcontroller; and transmitting one or more control signals from the reporting device to a lift apparatus based on the processed information regarding the measured at least one physiological parameter of the subject. In an aspect, the method further includes determining whether the measured at least one physiological parameter of the subject falls within the stored range of acceptable physiological parameter values; and transmitting one or more control signals from the reporting device to the lift apparatus to control operation of the lift apparatus if the measured at least one physiological parameter of the subject fails to fall within the stored range of acceptable physiological parameter values.

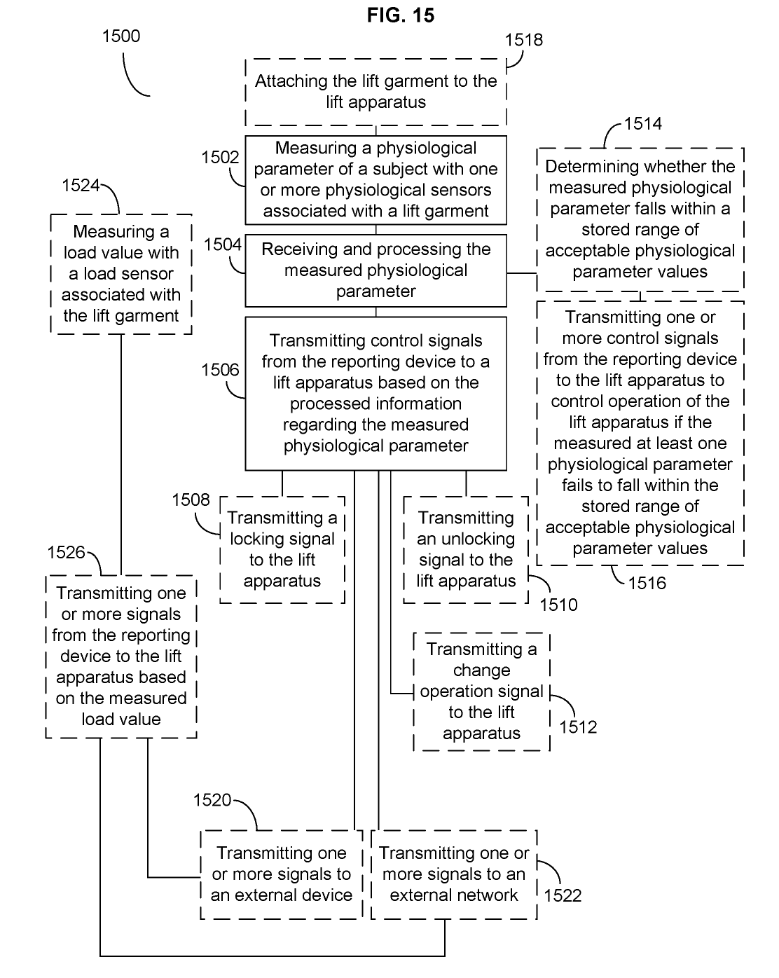

FIG. 15 is a flow diagram illustrating aspects of method 1500 implemented with a lift garment. The lift garment of method 1500 includes a fabric-like material, one or more physiological sensors, one or more lift attachment sites, at least one lift attachment element, a microcontroller including circuitry, and a reporting device. Step 1502 of method 1500 includes measuring a physiological parameter of a subject with one or more physiological sensors associated with a lift garment. In an aspect, method 1500 includes measuring the at least one physiological parameter of the subject with one or more physiological sensors attached to the lift garment. In an aspect, method 1500 includes measuring the at least one physiological parameter of the subject with one or more physiological sensors incorporated into the lift garment. In an aspect, method 1500 includes measuring at least one of heart rate, blood pressure, temperature, respiration, or biochemistry of the subject. In an aspect, method 1500 includes measuring the at least one physiological parameter of the subject with at least one of a heart rate sensor, a blood pressure sensor, a respiration sensor, a temperature sensor, or a biochemical sensor associated with the lift garment. In an aspect, method 1500 includes measuring oxygen saturation of the subject with one or more blood oxygenation sensors associated with the lift garment.

In an aspect, method 1500 includes in step 1504 receiving and processing the measured physiological parameter and in step 1506 transmitting control signals from the reporting device to a lift apparatus based on the processed information regarding the measured physiological parameter. In an aspect, method 1500 further includes in step 1514 determining whether the measured physiological parameter falls within a stored range of acceptable physiological parameter values, and in step 1516 transmitting control signals from the reporting device to the lift apparatus to control operation of the lift apparatus if the measured at least one physiological parameter fails to fall within the stored range of acceptable physiological parameter values. In an aspect, method 1500 includes transmitting the one or more control signals to the lift apparatus with a transmission unit including an antenna associated with the lift garment. In some embodiments, the transmission unit is associated with at least one of the microcontroller or the reporting device of the lift garment.

In some embodiments, method 1500 includes in step 1508 transmitting a locking signal to the lift apparatus. In an aspect, the method includes transmitting a locking signal from the reporting device to the lift apparatus if the measured at least one physiological parameter of the subject fails to fall within the range of acceptable physiological parameter values. In some embodiments, method 1500 includes in step 1510 transmitting an unlocking signal to the lift apparatus. In an aspect, the method includes transmitting an unlocking signal from the reporting device to the lift apparatus if the measured at least one physiological parameter of the subject falls within the range of acceptable physiological parameter values. In some embodiments, method 1500 includes in step 1512 transmitting a change operation signal to the lift apparatus. In an aspect, the method includes transmitting one or more change operation signals from the reporting device to the lift apparatus. In an aspect, transmitting the one or more change operation signals from the reporting device to the lift apparatus includes transmitting at least one of a block operation signal, a partially reverse operation signal, a fully reverse operation signal, a slow operation signal, or an accelerate operation signal from the reporting device to the lift apparatus. In an aspect, the method includes transmitting at least one of an on/off signal, an up/down signal, a speed signal, or an acceleration signal.

In some embodiments, method 1500 includes step 1520 of transmitting one or more signals to an external device. In an aspect, the method includes transmitting one or more signals indicative of the processed information regarding the measured at least one physiological parameter of the subject from the lift garment to an external device. In an aspect, the external device includes at least one of the lift apparatus, a mobile communication device, or a computing device. In some embodiments, method 1500 includes step 1522 of transmitting one or more signals to an external network. In an aspect, the method includes transmitting one or more signals indicative of the processed information regarding the measured at least one physiological parameter of the subject from the lift garment to an external network. In an aspect, the external network includes a health provider network. In an aspect, the health provider network includes the subject's electronic medical file. In an aspect, method 1500 further includes transmitting at least one of an optical signal, an audio signal, or a haptic signal from the reporting device of the lift garment indicative of the processed information regarding the measured at least one physiological parameter of the subject.

In some embodiments, method 1500 includes measuring at least one physiological parameter of the subject predictive of hypoxia with the one or more physiological sensors; receiving and processing information associated with the measured at least one physiological parameter of the subject predictive of hypoxia; and transmitting one or more control signals from the reporting device to the lift apparatus to control operation of the lift apparatus if the processed information associated with the measured at least one physiological parameter of the subject predictive of hypoxia indicates that the subject is experiencing hypoxia. In an aspect, method 1500 includes measuring oxygen saturation of the subject with one or more blood oxygenation sensors associated with the lift garment.

In some embodiments, method 1500 includes measuring the at least one physiological parameter of the subject at a first time point and at a second time point and assessing the rate of change. In an aspect, method 1500 includes receiving and processing information regarding measurement of the at least one physiological parameter of the subject at a first time point; receiving and processing information regarding measurement of the at least one physiological parameter of the subject at a second time point; determining if a rate of change between the measurement at the first time point and the measurement at the second time point falls within a range of acceptable rate changes; and transmitting a control signal from the reporting device to the lift apparatus to control operation of the lift apparatus if the rate of change between the measurement at the first time point and the measurement at the second time point fails to fall within the range of acceptable rate changes. For example, the method can include measuring heart rate of the subject at a first time point and heart rate of the subject at a second time point and determining if a change in heart rate is sufficiently problematic to warrant altering the operation of a lift apparatus.

Method 1500 further includes step 1518 of attaching the lift garment to the lift apparatus. In an aspect, method 1500 includes attaching the lift garment to the lift apparatus with the at least one lift attachment element associated with the fabric-like material of the lift garment. In an aspect, method 1500 includes attaching the lift garment to the lift apparatus with at least one of a hook, a loop of material, or a magnet associated with the fabric-like material.

In some embodiments, method 1500 includes step 1524 of measuring a load value with a load sensor associated with the lift garment. In an aspect, method 1500 includes measuring a load value with a load sensor associated with the lift garment at at least one of the one or more lift attachment sites or at at least one load path between the one or more lift attachment sites. In an aspect, the method includes measuring the load with a force transducer associated with the lift garment at at least one of the one or more lift attachment sites or at at least one load path between the one or more lift attachment sites. In an aspect, the method includes measuring the load value with at least one of a strain sensor, a stretch sensor, or a pressure sensor associated with the lift garment at at least one of the one or more lift attachment sites or at at least one load path between the one or more lift attachment sites. Method 1500 further includes in step 1526 transmitting one or more control signals from the reporting device to the lift apparatus based on the measured load value. In an aspect, the method further includes transmitting one or more signals including information regarding the measured load value from the reporting device to an external device, e.g., the lift apparatus, a mobile communication device, and/or a computing device. In an aspect, the method further includes transmitting one or more signals including information regarding the measured load value from the reporting device to an external network, e.g., a health provider network. In an aspect, the method includes transmitting at least one of an optical signal, an audio signal, or a haptic signal from the reporting device based on the measured load value.

A method of controlling a lift apparatus based on a measured physiological parameter of a subject is described. In an embodiment, a method implemented with a microcontroller includes receiving one or more signals indicative of a measured physiological parameter of a subject from one or more physiological sensors; determining whether the measured physiological parameter of the subject falls within a range of acceptable physiological parameter values stored in the microcontroller; and transmitting one or more control signals from a transmission unit operably coupled to the microcontroller to a lift apparatus to control operation of the lift apparatus based on whether the measured physiological parameter of the subject falls within the range of acceptable physiological parameter values. In an aspect, the microcontroller, the transmission unit, and the one or more physiological sensors are part of a single monitoring unit. In an aspect, the microcontroller and the transmission unit are a separate unit from the one or more physiological sensors. In an aspect, the method includes receiving one or more signals indicative of at least one of a measured heart rate, a measured blood pressure, a measured respiration rate, a measured body temperature, a measured oxygen saturation, or a measured biochemical analyte. In an aspect, the method includes receiving one or more signals indicative of the measured physiological parameter from at least one of a heart rate sensor, a blood pressure sensor, a respiration sensor, a temperature sensor, a blood oxygenation sensor, or a biochemical sensor. In an aspect, the method includes transmitting at least one of an on/off, an up/down, a speed, or an acceleration signal from the transmission unit operably coupled to the microcontroller to the lift apparatus to control operation of the lift apparatus based on whether the measured physiological parameter of the subject falls within the range of acceptable physiological parameter values.

Described herein are devices, systems, and methods for use in lifting a subject. In an aspect, a wearable lift device is described for use with a lift apparatus, e.g., a Hoyer-like lift. In some embodiments, the wearable lift device is designed for continuous or long-term wear by a subject to allow for convenient transfer of the subject with a lift apparatus from one position or place to another position or place. In some embodiments, the wearable lift device is configured for use with a lift apparatus to aid in transferring a subject from one bed to another bed. In some embodiments, the wearable lift device is configured for use with a lift apparatus to aid in transferring a subject from a bed to a chair. In some embodiments, the wearable lift device is configured for use with a lift apparatus to aid in helping a subject reach a standing position. In some embodiments, the wearable lift device is configured for use with a lift apparatus to aid in toileting a subject. In an aspect, the wearable lift device is configured for use in a hospital, skilled nursing, or assisted living facility. For example, the wearable lift device can be configured for use in transferring a patient in a hospital or skilled nursing facility from a bed to a wheelchair. In an aspect, the wearable lift device is configured for use in a residential setting. For example, the wearable lift device can be configured for use in lifting a subject who has fallen on the floor in an assisted or independent living facility. For example, the wearable lift device can be configured for transferring a limited mobility subject from one position to another in a residential setting. In some embodiments, the wearable lift device is configured such that the subject is able to perform the transfer procedure unaided, i.e., in the absence of a caregiver, allowing for increased independence. The lift apparatus can include a floor or mobile lift apparatus, a ceiling lift apparatus, a stand assist lift apparatus, and/or a wall lift apparatus.

In some embodiments, a wearable lift device is designed for suspending/supporting a subject outside of a clinical or medical setting. For example, a wearable lift device, such as described herein, is contemplated for use mountain/rock climbing and/or caving; helicopter rescue; safety gear used for certain occupations that involve working at elevation, e.g., window washers, house painters, utility pole repair persons, roofers, builders, and the like.

In some embodiments, a wearable lift device includes a load sensor configured to measure a load. For example, the wearable lift device can include a load sensor configured to measure the load of a subject as he or she is wearing the wearable lift device and lifted/suspended by a lift apparatus. For example, one or more load sensors associated with the wearable lift device can be used to determine whether the load of the subject is distributed appropriately, e.g., evenly, in the wearable lift device.

In some embodiments, a wearable lift device includes one or more physiological sensors configured to measure at least one physiological parameter of a subject. For example, the wearable lift device can include one or more physiological sensors configured to measure at least one physiological parameter of the subject predictive of hypoxia, e.g., heart rate, blood oxygenation, blood pressure, and/or respiration rate. For example, the wearable lift device can include one or more physiological sensors configured to measure a physiological symptom of suspension trauma, e.g., altered heart and/or respiration rate, changes in blood pressure and/or oxygenation, and the like.

Described herein are aspects of wearable lift devices. In some embodiments, a wearable lift device includes a flexible material having a shape sufficient to substantially completely encircle at least a portion of a subject's body and including one or more lift attachment sites; at least one fastener configured to secure the flexible material around the at least a portion of the subject's body; at least one lift attachment element associated with the flexible material at one or more lift attachment sites, the at least one lift attachment element configured to attach the wearable lift device to a lift apparatus; a load sensor configured to measure a load, the load sensor associated with at least one of the one or more lift attachment sites or along a load path between the one or more lift attachment sites; a microcontroller including circuitry configured to receive and process information regarding the measured load; and a reporting device operably coupled to the microcontroller and configured to transmit one or more signals indicative of the processed information regarding the measured load.

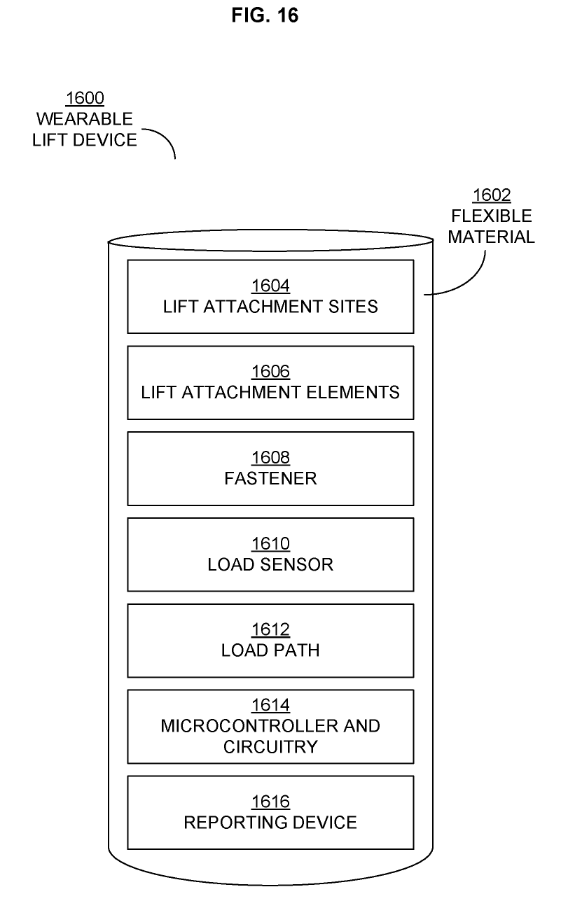

FIG. 16 illustrates aspects of a wearable lift device 1600. Wearable lift device 1600 includes a flexible material 1602 having a shape sufficient to substantially completely encircle at least a portion of a subject's body. Flexible material 1602 further includes one or more lift attachment sites 1604. In some embodiments, the flexible material 1602 is shaped to substantially completely encircle at least a portion of the subject's arms and legs. In some embodiments, the flexible material 1602 is shaped to substantially completely encircle the subject's torso. In some embodiments, the flexible material 1602 is shaped to substantially completely encircle a torso and at least a portion of arms and legs of the subject. In an aspect, the flexible material 1602 is shaped to substantially completely encircle at least a portion of a human body.

In an aspect, flexible material 1602 having a shape sufficient to substantially completely encircle the at least a portion of the subject's body includes one or more interconnected straps. In an aspect, flexible material 1602 having a shape sufficient to completely encircle the at least a portion of the subject's body includes webbing. In an aspect, flexible material 1602 having a shape sufficient to completely encircle the at least a portion of the subject's body includes an article of clothing. In some embodiments, at least a portion of the flexible material 1602 includes a woven or knit material. For example, the flexible material can include one or more woven or knitted interconnected straps. In some embodiments, at least a portion of the flexible material 1602 includes a non-woven material. For example, the flexible material can include webbing formed with neoprene. In some embodiments, at least a portion of the flexible material 1602 is formed from at least one polymer type. In an aspect at least one of the at least one load sensor, the microcontroller including circuitry, or the reporting device is woven, knitted, laminated, printed, or stitched into or onto the flexible material. Non-limiting aspects of incorporating electronics into wearable materials is presented above herein.

Wearable lift device 1600 further includes at least one lift attachment element 1606 associated with the flexible material 1602 at at least one of the one or more lift attachment sites 1604. The at least one lift attachment element 1606 is configured to attach the wearable lift device 1600 to a lift apparatus. For example, the lift attachment element can be configured to attach or otherwise engage with a spreader bar, a cradle, or similar attachment component of a lift apparatus. In an aspect, the lift attachment element is attached to the flexible material. For example, the lift attachment element can be glued, stapled, bolted, or sewn to the flexible material. In an aspect, the lift attachment element 1606 is incorporated into the flexible material 1602. For example, the lift attachment element can be an extension of the flexible material (e.g., a loop of the flexible material).

Wearable lift device 1600 further includes at least one fastener 1608 configured to secure the flexible material 1602 around the at least a portion of the subject's body. Wearable lift device 1600 further includes a load sensor 1610 configured to measure a load. Load sensor 1610 is associated with at least one of the one or more lift attachment sites 1604 or along a load path 1612 between the one or more lift attachment sites 1604. Wearable lift device 1600 further includes a microcontroller 1614 including circuitry configured to receive and process information regarding the measured load. Wearable lift device 1600 further includes a reporting device 1616 operably coupled to microcontroller 1614 and configured to transmit one or more signals indicative of the processed information regarding the measured load value. In some embodiments, microcontroller 1614 includes circuitry configured to receive and process the information regarding the measured load and determine whether the measured load falls within a range of acceptable load values, and reporting device 1616 operably coupled to microcontroller 1614 is configured to transmit a control signal to the lift apparatus to control operation of the lift apparatus based on whether the measured load falls within the range of acceptable load values. In an aspect, reporting device 1616 operably coupled to microcontroller 1614 is configured to transmit at least one of an on/off control signal, an up-down control signal, a speed signal, or an acceleration signal to the lift apparatus based on whether the measured load falls within the range of acceptable load values.

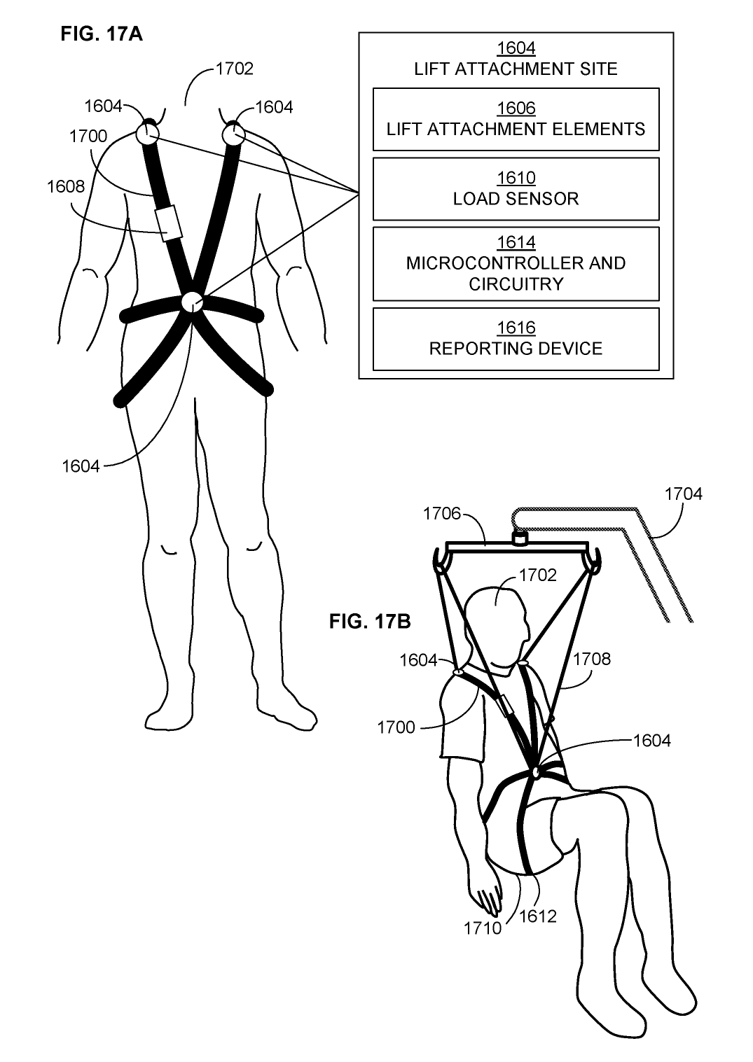

FIGS. 17A and 17B illustrate further aspects of a wearable lift device. FIG. 17A shows an embodiment of a wearable lift device 1700 being worn by subject 1702. In this non-limiting example, the flexible material of the wearable lift device 1700 is shaped with multiple bands or straps encircling portions (e.g., waist, hips, and neck) of the body of subject 1702. Wearable lift device 1700 further includes lift attachment site 1604. In this non-limiting example, wearable lift device 1700 includes three lift attachment sites 1604. However, as few as one lift attachment site or as many as twenty lift attachment sites are contemplated, depending upon the configuration and size of the wearable lift device and the number and type of attachments portion(s) on the lift apparatus (e.g., spreader bar(s), cradle(s), and the like). Wearable lift device 1700 further includes at least one fastener 1608, e.g., a buckle or cinch, configured to secure the flexible material of wearable lift device 1700 around the at least a portion of the body of subject 1702. The lift attachment sites 1604 of wearable lift device 1700 include at least one lift attachment element 1606, e.g., a hook, a loop of material, or a magnet. In this non-limiting example, the lift attachment site 1604 is also the site for other components of the wearable lift device. Lift attachment site 1604 of wearable lift device 1700 includes a load sensor 1610 configured to measure a load associated with the lift attachment site 1604. In some embodiments, the load sensor 1610 is not co-localized with the lift attachment site, but associated with the flexible material elsewhere in the wearable lift device. For example, one or more load sensors can be positioned along a load path between one or more lift attachment sites. Wearable lift device 1700 further includes a microcontroller 1614 including circuitry and reporting device 1616 at each of the lift attachment sites to receive, process, and transmit information regarding the measured load at a specific lift attachment site. In some embodiments, a central microcontroller and reporting device receive, process, and transmit information regarding the measured load at each of one or more lift attachment sites or along a load path between the one or more lift attachment sites.

FIG. 17B shows wearable lift device 1700 being worn by subject 1702 and attached to lift apparatus 1704. In an aspect, lift apparatus 1704 includes a Hoyer-like lift apparatus. Wearable lift device 1700 is attached to an attachment portion 1706 of lift apparatus 1704 through straps 1708 connected to the lift attachment elements, e.g., hooks, loops of material, or magnets associated with the lift attachment sites 1604. Also shown in FIG. 17B is a non-limiting example of a load path 1612 exerted by a portion of the flexible material forming wearable lift device 1700 and extending along the buttocks 1710 of subject 1702 between lift attachment sites 1604.

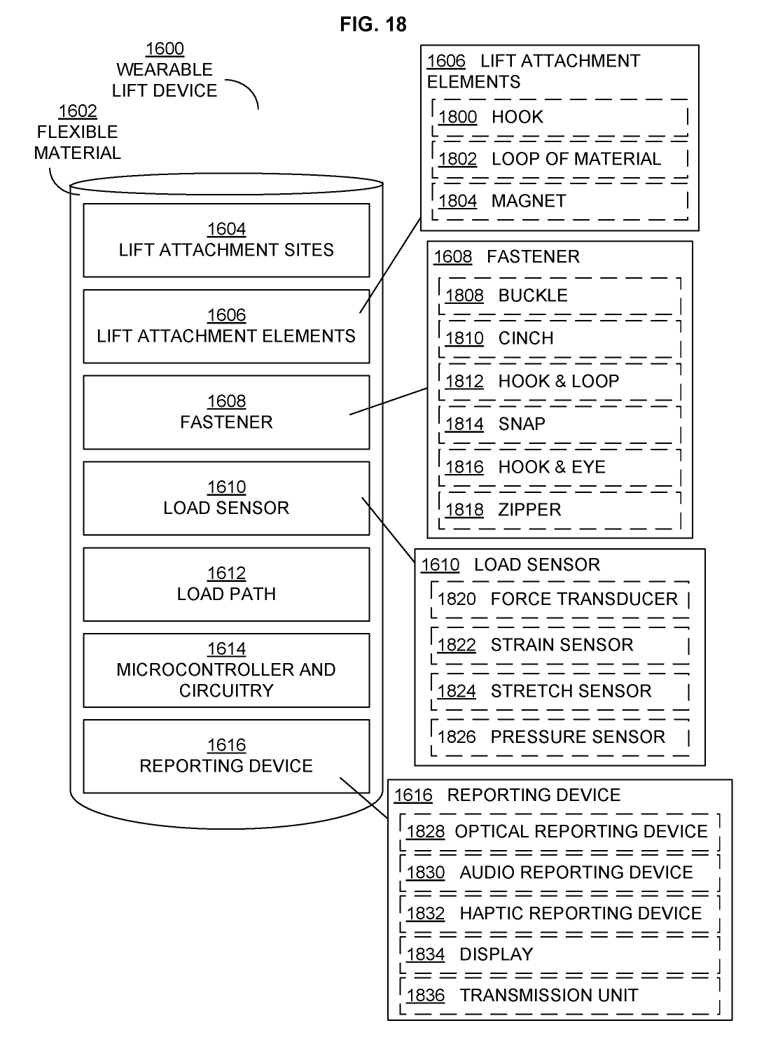

FIG. 18 illustrates further aspects of a wearable lift device. Wearable lift device 1600 includes one or more lift attachment sites 1604. In an aspect, at least one of the one or more lift attachment sites 1604 is where the one or more lift attachment elements 1606 are attached and/or incorporated to the flexible material 1602 of the wearable lift device 1600. In an aspect, each of the lift attachment sites 1604 includes at least one lift attachment element 1606. In an aspect, at least one of the one or more lift attachment sites 1604 includes a load sensor 1610 associated with the wearable lift device 1600. In an aspect, each of the one or more lift attachment sites 1604 includes at least one load sensor 1610 associated with the wearable lift device 1600.

Wearable lift device 1600 includes at least one lift attachment element 16506 associated with the flexible material 1602 at at least one of the one or more lift attachment sites 1604. In some embodiments, the at least one lift attachment element 1606 includes at least one of a hook 1800 or a loop of material 1802. In some embodiments, the at least one lift attachment element 1604 includes a magnet 1804. Non-limiting aspects of lift attachment elements have been described above herein.

Returning to FIG. 18, wearable lift device 1600 includes at least one fastener 1608 configured to secure the flexible material 1602 around the at least a portion of the subject's body. In an aspect, the at least one fastener 1608 is configured to secure the flexible material 1602 tightly around the at least a portion of the subject's body. In an aspect, the at least one fastener 1608 includes at least one buckle 1808. In an aspect, the at least one fastener 1608 includes at least one cinch 1810. In an aspect, the at least one fastener 1608 includes at least one of a hook and loop fastener 1812, a snap fastener 1814, a hook and eye fastener 1816, or a zipper 1818. In an aspect, the wearable lift device includes a single, centrally located fastener. For example, a single fastener can be used to fasten together multiple interconnected straps. In an aspect, the wearable lift device includes multiple fasteners which may or may not be co-localized with the lift attachment sites.

Returning to FIG. 18, wearable lift device 1600 further includes a load sensor 1610 configured to measure a load, the load sensor 1610 associated with at least one of the one or more lift attachment sites 1604 or along a load path 1612 between the one or more lift attachment sites 1604. In some embodiments, the load sensor 1610 includes a force transducer 1820. In some embodiments, the load sensor 1610 includes at least one of a strain sensor 1822, a stretch sensor 1824, or a pressure sensor 1826. In some embodiments, two or more load sensors 1610 are distributed along a length of the at least one load path 1612. Non-limiting aspects of load sensors are described above herein.

Wearable lift device 1600 includes microcontroller 1614 including circuitry configured to receive and process information regarding the measured load. The microcontroller can include a microprocessor, a central processing unit (CPU), a digital signal processor (DSP), application-specific integrated circuit (ASIC), a field programmable gate entry (FPGA), or the like, or any combinations thereof, and can include discrete digital or analog circuit elements or electronics, or combinations thereof. The microcontroller can further include signal processing algorithms, e.g., band pass filters, low pass filters, or any other single processing algorithms or combinations thereof.

Microcontroller 1614 further includes some form of accessible memory. In an aspect, the microcontroller includes RAM (volatile memory) for data storage. In an aspect, the microcontroller includes ROM, EPROM, EEPROM, or flash memory for program and operating parameter storage. The memory component can be used to store algorithms, subject data, and reference range data, e.g., a range of acceptable load values, a range of acceptable physiological parameter values, or a range of acceptable oxygen saturation levels. The microcontroller further includes in/out (I/O) ports for receiving information, e.g., signals from one or more sensors, and transmitting information, e.g., signals to the reporting device. In an aspect, the microcontroller further includes a clock generator, analog-to-digital convertors, serial ports, and/or data bus to carry information. In an aspect, the microcontroller includes a small integrated chip attached to or incorporated into the lift garment.

In some embodiments, microcontroller 1614 includes a stored range of acceptable load values and circuitry configured to determine if the measured load falls within the range of acceptable load values. The stored range of acceptable load values can be specific to the subject and the various load points or paths associated with the wearable lift device when worn by the subject and attached to a lift apparatus. The stored range of acceptable load values can be specific to the wearable lift device, e.g., a small, medium, large, extra-large, plus sized wearable lift device. The stored range of acceptable load values can be specific to a shape of the wearable lift device. The stored range of acceptable load values can be specific to the number of lift attachment sites and/or distribution of load paths between the one or more lift attachment sites.

In embodiments, a wearable lift device 1600 includes a power source configured to provide power to one or more components of the wearable lift device including, but not limited to, one or more sensor types, the microcontroller, and/or the reporting device. In an aspect, the power source includes a wired connection to a standard electrical outlet. In an aspect, the power source is associated with the lift apparatus to which the wearable lift device worn by the subject is attached. In an aspect, the power source is a resident device component associated with the wearable lift device. Non-limiting examples of resident device components include batteries (e.g., a camera or watch-sized alkaline, lithium, or silver-oxide battery, a thin film battery, a microbattery) and solar cells (e.g., silicon-based solar cells) configured to convert light energy into electrical energy for use by components of the lift garment. In an aspect, the power source includes one or more components positioned remotely from the wearable lift device that transmit power signals via associated wireless power methods including, but not limited to, inductive coupling of power signals. In an aspect, the wearable lift device receives power through an energy harvesting unit capable of converting received electromagnetic energy into electrical energy. For example, the wearable lift device can receive power through energy harvesting from body heat, breathing, or body movement (e.g., walking).

With reference to FIG. 18, wearable lift device 1600 includes reporting device 1616 operably coupled to microcontroller 1614 and configured to transmit one or more signals indicative of the processed information regarding the measured load. In an aspect, the reporting device 1616 is configured to transmit one or more optical signals, audio signals or haptic signals indicative of the processed information regarding the measured load. In an aspect, the reporting device 1616 is configured to transmit one or more wireless signals indicative of the processed information regarding the measured load. In an aspect, reporting device 1616 includes an optical reporting device 1828. For example, the optical reporting device can include one or more lights, e.g., LEDs. In an aspect, the optical reporting device 1828 includes one or more color-coded lights. In an aspect, reporting device 1616 includes an audio reporting device 1830 including a at least one speaker. For example, the audio reporting device can include a sound card with a speaker that is attached to the wearable lift device. In an aspect, reporting device 1616 includes a haptic reporting device 1832. For example, the haptic reporting device can include a vibrating element. In an aspect, reporting device 1616 includes a display 1834. In an aspect, reporting device 1616 includes a transmission unit 1836 including an antenna.

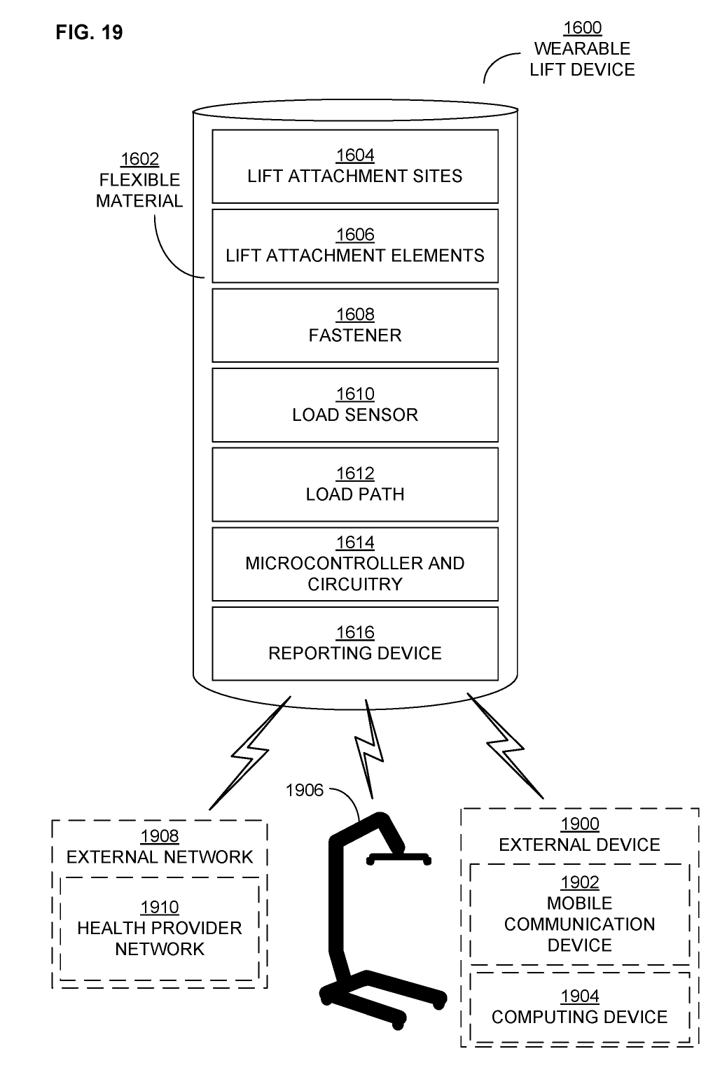

FIG. 19 illustrates further aspects of wearable lift device 1600. In some embodiments, the reporting device 1616 of wearable lift device 1600 is configured to communicate with an external device 1900. For example, the reporting device can be configured to transmit one or more signals including information regarding the measured load to an external device, e.g., a smart phone or tablet computer. In an aspect, reporting device 1616 is configured to wirelessly communicate with external device 1900. In some embodiments, reporting device 1616 is configured to communicate with a mobile communication device 1902. In some embodiments, reporting device 1616 is configured to communicate with a computing device 1904. In some embodiments, reporting device 1616 is configured to communicate with a lift apparatus 1906. For example, the reporting device can be configured to transmit one or more control signals to control an operation of the lift apparatus. In some embodiments, the reporting device 1616 of wearable lift device 1600 is configured to communicate with an external network 1908. In an aspect, reporting device 1616 is configured to wirelessly communicate with external network 1908. In some embodiments, external network 1908 includes a health provider network 1910. For example, the reporting device can be configured to wirelessly communicate with the subject's electronic medical file stored on a health provider network.

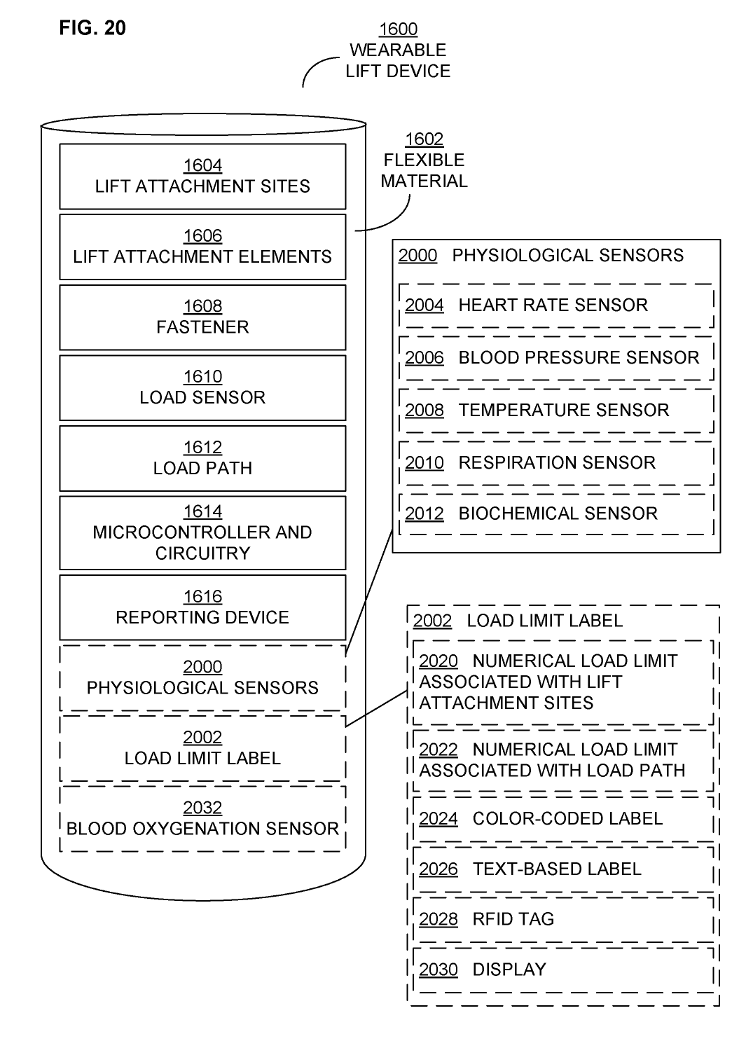

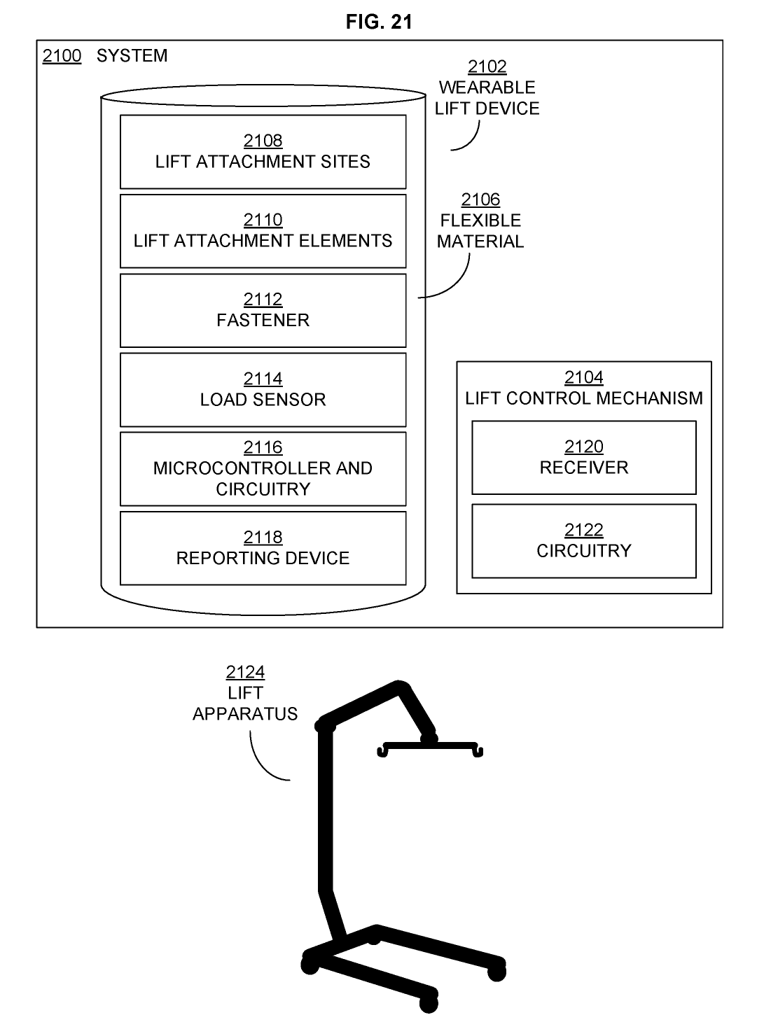

FIG. 20 illustrates further aspects of wearable lift device 1600. In some embodiments, wearable lift device 1600 includes one or more physiological sensors 2000 configured to measure at least one physiological parameter of the subject. In an aspect, the one or more physiological sensors 2000 are attached to at least one surface of the flexible material 1602. In an aspect, the one or more physiological sensors 2000 are incorporated into at least one surface of flexible material 1602. In an aspect, the one or more physiological sensors 2000 are woven, knitted, laminated, printed, or stitched into or onto the flexible material 1602.

In an aspect, at least one of the one or more physiological sensors 2000 includes at least one of a heart rate sensor 2004, a blood pressure sensor 2006, a temperature sensor 2008, a respiration sensor 2010, or a biochemical sensor 2012. In some embodiments, at least one of the one or more physiological sensors 2000 is configured to measure at least one physiological parameter of the subject predictive of hypoxia. For example, physiological sensors can measure heart rate, blood pressure, respiration, or oxygen saturation of the subject as a measure of hypoxia. Non-limiting examples of physiological sensors have been described above herein.

In some embodiments, the microcontroller 1614 includes circuitry configured to receive and process information regarding the measured at least one physiological parameter of the subject; and reporting device 1616 is configured to transmit the processed information regarding the measured at least one physiological parameter of the subject. In an aspect, the reporting device 1616 is configured to transmit the processed information regarding the measured at least one physiological parameter of the subject to an external device, e.g., a mobile communication device and/or a computing device. In an aspect, the reporting device 1616 is configured to transmit the processed information regarding the measured at least one physiological parameter of the subject to an external network, e.g., a health provider network.

In some embodiments, wearable lift device 1600 includes at least one blood oxygenation sensor 2032. For example, the wearable lift device can include at least one blood oxygenation sensor configured to measure a subject's blood oxygen saturation before, during, and/or after a lift/transfer procedure. In an aspect, the blood oxygenation sensor 2032 is associated with the at least one load path 1612. For example, the wearable lift device can include one or more blood oxygenation sensors associated with at least one of the load paths between the one or more lift attachment sites to measure oxygen saturation of the subject while the wearable lift device is in use with a lift apparatus. For example, the process of lifting a subject may inadvertently alter blood supply during a lift/transfer procedure due to pressure put on one or more portions of the subject's body along a load path. In an aspect, the at least one blood oxygenation sensor 2032 includes a near infrared optical blood oxygenation sensor. In an embodiment, the at least one blood oxygenation sensor 2032 is associated with a surface of the flexible material 1602 configured for placement in contact with an external surface of the subject. For example, the blood oxygenation sensor can be positioned on an inside portion of the flexible material of the wearable lift device that comes in direct contact with the skin surface of a human subject. In an aspect, microcontroller 1614 includes circuitry configured to receive and process the measured oxygen saturation of the subject, and reporting device 1616 is configured to transmit one or more signals indicative of the processed information regarding the measured oxygen saturation of the subject. For example, the reporting device can transmit one or more signals indicative of the process information regarding the measured oxygen saturation of the subject through an optical reporting device, an audio reporting device, a haptic reporting device, or a display associated with the wearable lift device. For example, the reporting device can transmit one or more signals indicative of the process information regarding the measured oxygen saturation of the subject through an antenna associated with a transmission unit to an external device (e.g., a mobile communication device or computing device) or an external network (e.g., a health provider network). For example, the reporting device can transmit one or more control signals to the lift apparatus to control an operation (e.g., on/off, up/down, speed, or acceleration) of the lift apparatus in response to the measured oxygen saturation of the subject.

Returning to FIG. 20, in some embodiments, wearable lift device 1600 further includes at least one load limit label 2002. In an aspect, the at least one load limit label 2002 includes a numerical load limit 2020 associated with each of the one or more lift attachment sites 1604. For example, the load limit label can display by way of color coding, text, or RFID information the maximum load associated with each of the lift attachment sites. In an aspect, the at least one load limit label 2002 includes a numerical load limit 2022 associated with the at least one load path 1612 between the one or more lift attachment sites 1604. For example, the load limit label can display by way of color coding, text, or RFID information the maximum load associated with a load path between lift attachment sites. In an aspect, the at least one load limit label 2002 includes at least one of a color-coded label 2024, a text-based label 2026, or an RFID (radiofrequency identification) tag 2028. In an aspect, the at least one load limit label 2002 includes a display 2030. Non-limiting aspects of load limit labels are described above herein.

Described herein is a system including a wearable lift device and a lift control mechanism. In some embodiments, a system includes a wearable lift device including a flexible material having a shape sufficient to substantially completely encircle at least a portion of a subject's body, at least one fastener configured to secure the flexible material around the at least a portion of the subject's body; at least one lift attachment element associated with the flexible material at one or more lift attachment sites, the at least one lift attachment element configured to attach the wearable lift device to a lift apparatus; a load sensor configured to measure a load, the load sensor associated with at least one of the one or more lift attachment sites or along at least one load path between the one or more lift attachment sites; a microcontroller including circuitry configured to receive and process information regarding the measured load; and a reporting device operably coupled to the microcontroller and configured to transmit one or more signals indicative of the processed information regarding the measured load; and a lift control mechanism including a receiver configured to receive the one or more signals from the reporting device indicative of the processed information regarding the measured load; and circuitry configured to control a lift function of the lift apparatus in response to the one or more signals received from the reporting device of the wearable lift device.

FIG. 21 illustrates aspects of a system including a wearable lift device and a lift control mechanism. System 2100 includes wearable lift device 2102 and lift control mechanism 2104. Wearable lift device 2102 includes flexible material 2106 having a shape sufficient to substantially completely encircle at least a portion of a subject's body. In an embodiment, flexible material 2106 of wearable lift device 2102 is shaped to substantially completely encircle at least a portion of the subject's arms and legs. In an embodiment, flexible material 2106 of wearable lift device 2102 is shaped to substantially completely encircle at least a portion of the subject's torso. In an embodiment, flexible material 2106 of wearable lift device 2102 is shaped to substantially completely encircle the subject's torso and a portion of the subject's arms and legs. In an aspect, flexible material 2106 of wearable lift device 2102 is shaped to substantially completely encircle at least a portion of a human body.

In an aspect, the flexible material 2106 having a shape sufficient to substantially completely encircle the at least a portion of the subject's body includes one or more interconnecting belts or straps. In an aspect, the flexible material 2106 having a shape sufficient to substantially completely encircle the at least a portion of the subject's body includes webbing. In an aspect, the flexible material 2106 having a shape sufficient to substantially completely encircle the at least portion of the subject's body includes an article of clothing. In an aspect, at least a portion of flexible material 2106 of wearable lift device 2102 includes a woven or knit material. In an aspect, at least a portion of flexible material 2106 of wearable lift device 2102 includes a non-woven material. In an aspect, at least a portion of flexible material 2106 of wearable lift device 2102 is formed from at least one polymer type.

The flexible material 2106 of wearable lift device 2102 includes one or more lift attachment sites 2108. The wearable lift device 2102 further includes at least one lift attachment element 2110 associated with the flexible material 2106 at the one or more lift attachment sites 2108. The at least one lift attachment element 2110 is configured to attach the wearable lift device 2102 of a lift apparatus 2124. In an aspect, the at least one lift attachment element 2110 of wearable lift device 2102 includes at least one of a hook, a loop of material, or a magnet.

Wearable lift device 2102 of system 2100 further includes at least one fastener 2112 configured to secure the flexible material 2106 around the at least a portion of the subject's body. One or more fasteners can be configured to tightly secure the flexible material around a portion of the subject's body. In an aspect, the at least one fastener 2112 of wearable lift device 2102 includes at least one of a buckle, a cinch, a hook and loop fastener, a hook and eye fastener, a snap, or a zipper.

Wearable lift device 2102 of system 2100 further includes a load sensor 2114 configured to measure a load. Load sensor 2114 is associated with at least one of the one or more lift attachment sites or along at least one load path between the one or more lift attachment sites. In an embodiment, wearable lift device 2102 includes a load sensor 2114 at each of the one or more lift attachment sites 2108. In an embodiment, wearable lift device 2102 includes two or more load sensors 2114 distributed along a length of the at least one load path between the one or more lift attachment sites 2108. In an embodiment, each of the one or more lift attachment sites 2108 and the at least one load path between the one or more lift attachment sites 2108 includes at least one load sensor 2114. In an aspect, the load sensor 2114 of wearable lift device 2102 includes a force transducer. In an aspect, the load sensor 2114 of wearable lift device 2102 includes at least one of a strain sensor, a stretch sensor, or a pressure sensor. Non-limiting aspects of load sensors have been described above herein.

In some embodiments, wearable lift device 2102 of system 2100 further includes at least one load limit label including a numerical load limit associated with at least one of each of the one or more lift attachment sites 2108 or the at least one load path between the one or more lift attachment sites 2108. In an aspect, the load limit label includes at least one of a color-coded label, a text-based label, or a radiofrequency identification tag. In an aspect, the load limit label includes a display.

Wearable lift device 2102 of system 2100 further includes microcontroller 2116 including circuitry configured to receive and process information regarding the measured load. In some embodiments, microcontroller 2116 includes circuitry configured to receive and process the information regarding the measured load and determine whether the measured load falls within a range of acceptable load values, and reporting device 2118 operably coupled to microcontroller 2116 is configured to transmit a control signal to the lift control mechanism 2104 to control operation of the lift apparatus 2124 based on whether the measured load falls within the range of acceptable load values. In an aspect, the range of acceptable load values is stored in the microcontroller 2116 of wearable lift device 2102. In an aspect, reporting device 2118 operably coupled to microcontroller 2116 is configured to transmit at least one of an on/off signal, an up-down signal, a speed signal, or an acceleration signal to the lift control mechanism 2104 based on whether the measured load falls within the range of acceptable load values. In an aspect, reporting device 2118 operably coupled to microcontroller 2116 is configured to transmit a locking signal to the lift control mechanism to lock the function of the lift apparatus 2124 if the measured load fails to fall within the range of acceptable load values.

Wearable lift device 2102 of system 2100 includes reporting device 2118 operably coupled to microcontroller 2116 and configured to transmit one or more signal indicative of the processed information regarding the measured load. In an aspect, reporting device 2118 of wearable lift device 2102 includes at least one of an optical reporting device, an audio reporting device, a haptic reporting device, or a display. In an aspect, reporting device 2118 of wearable lift device 2102 includes a transmission unit including an antenna. In an aspect, reporting device 2118 of wearable lift device 2102 is configured to communicate with an external device. For example, the reporting device of the wearable lift device can be configured to communicate with a lift control mechanism located on the lift apparatus. For example, the reporting device of the wearable lift device can be configured to communicate with a mobile communication device or a computing device. In an aspect, reporting device 2118 of wearable lift device 2102 is configured to communicate with an external network. In an aspect, the external network includes a health provider network. For example, the reporting device of the wearable lift device can be configured to communicate with a health provider network that includes the subject's electronic medical or healthcare file.

System 2100 further includes lift control mechanism 2104. Lift control mechanism 2104 includes a receiver 2120 configured to receive the transmitted one or more signals from reporting device 2118 indicative of the processed information regarding the measured load, and circuitry 2122 configured to control a function of the lift apparatus 2124 in response to the one or more signals received from the reporting device 2118 of the wearable lift garment 2102. The function can include at least one of an on/off function, an up/down function, a speed function, and/or an acceleration function of the lift apparatus. In some embodiments, lift control mechanism 2104 is associated with the wearable lift device 2102 and configured to wirelessly communicate with the lift apparatus 2124. For example, the circuitry of the lift control mechanism can be configured to wirelessly transmit a signal to control a function of the lift apparatus. In this non-limiting embodiment, the lift apparatus includes a receiver capable of receiving the wirelessly transmitted control signal (e.g., a Bluetooth or similar signal type) from the lift control mechanism. In some embodiments, lift control mechanism 2104 is associated with the lift apparatus 2124 and configured to wirelessly communicate with the wearable lift device 2102. For example, the receiver of the lift control mechanism can be configured to wirelessly receive the one or more signals from the reporting device of the wearable lift device indicative of the processed information regarding the measured load, and the circuitry configured control a function of the lift apparatus in response to the one or more signal received from reporting device of the wearable lift device. The circuitry can be configured to transmit a control signal through either a wired or wireless communication link.

In some embodiments, the wearable lift device 2102 of system 2100 further includes one or more physiological sensors incorporated into the wearable lift device and configured to measure at least one physiological parameter of the subject. In an aspect, the one or more physiological sensors include at least one of a heart rate sensor, a blood pressure sensor, a respiration sensor, a temperature sensor, or a biochemical sensor. In an aspect, at least one of the one or more physiological sensors incorporated into the wearable lift device is configured to measure at least one physiological parameter of the subject predictive of hypoxia. In an aspect, at least one of the one or more physiological sensors includes at least one blood oxygenation sensor configured to measure an oxygen saturation level of the subject. In an aspect, the at least one blood oxygenation sensor includes a near infrared blood oxygenation sensor. In as aspect, the at least one blood oxygenation sensor is attached to a surface of the wearable lift device 2102 intended to come in contact with an external surface of the subject. In an aspect, the microcontroller 2116 of wearable lift device 2102 includes circuitry configured to receive and process information regarding the measured at least one physiological parameter of the subject, and the reporting device 2118 includes circuitry configured to transmit one or more signals indicative of the processed information regarding the at least one physiological parameter of the subject. For example, the reporting device can be configured to transmit one or more signals indicative of the processed information regarding the at least one physiological parameter of the subject to an external device, e.g., a mobile communication device or a computing device, or to an external network, e.g., a health provider network.

Described herein are aspects of a lift sling and a method of use. In an aspect, a lift sling is described for use with a lift apparatus, e.g., a Hoyer-like lift. In some embodiments, the lift sling is configured for use with a lift apparatus to aid in transferring a subject from one bed to another bed. In some embodiments, the lift sling is configured for use with a lift apparatus to aid in transferring a subject from a bed to a chair. In some embodiments, the lift sling is configured for use with a lift apparatus to aid in helping a subject reach a standing position. In an aspect, the lift sling includes a commode hole for use in toileting. In an aspect, the lift sling is configured for use in a hospital, skilled nursing, or assisted living facility. For example, the lift sling can be configured for use in transferring a patient in a hospital or skilled nursing facility from a bed to a wheelchair. In an aspect, the lift sling is configured for use in a residential setting. For example, the lift sling can be configured for use in lifting a subject who has fallen on the floor in an assisted or independent living facility. For example, the lift sling can be configured for transferring a limited mobility subject from one position to another in a residential setting. In some embodiments, the lift sling is reusable for by different subjects following proper cleaning/sterilization. In some embodiments, the lift sling is disposable and intended to be discarded after use by a given subject. In some embodiments, the lift sling is configured such that the subject is able to perform the transfer procedure unaided, i.e., in the absence of a caregiver, allowing for increased independence. The lift apparatus can include a floor or mobile lift apparatus, a ceiling lift apparatus, a stand assist lift apparatus, and/or a wall lift apparatus.

In some embodiments, a lift sling is designed for suspending/supporting a subject outside of a clinical setting. For example, a lift sling, such as described herein, is contemplated for use mountain/rock climbing and/or caving; helicopter rescue; safety gear used for certain occupations that involve working at elevation—window washers, house painters, utility pole repair persons, roofers, builders, and the like.

In some embodiments, a lift sling includes one or more physiological sensors configured to measure at least one physiological parameter of a subject. For example, the lift sling can include one or more physiological sensors configured to measure at least one physiological parameter of the subject predictive of hypoxia, e.g., heart rate, blood oxygenation, blood pressure, and/or respiration rate. For example, the lift sling can include one or more physiological sensors configured to measure a physiological symptom of suspension trauma, e.g., altered heart and/or respiration rate, changes in blood pressure and/or oxygenation, and the like. In some embodiments, a lift sling includes a load sensor configured to measure a load. For example, the lift sling can include a load sensor configured to measure the load of a subject as he or she is sitting or lying in the lift sling and lifted by a lift apparatus. For example, one or more load sensors associated with the lift sling can be used to determine whether the load of the subject is distributed appropriately, e.g., evenly, in the lift sling.

Described herein are aspects of a lift sling and a method of use. In some embodiments, a lift sling includes a flexible material having a shape sufficient to at least partially encircle a portion of a subject's body; at least one lift attachment element associated with the flexible material at one or more lift attachment sites, the at least one lift attachment element configured to attach the lift sling to a lift apparatus; one or more physiological sensors configured to measure at least one physiological parameter of the subject; a microcontroller including circuitry configured to receive and process information regarding the measured at least one physiological parameter of the subject; and a reporting device operably coupled to the microcontroller and configured to transmit one or more signals indicative of the processed information regarding the measured at least one physiological parameter of the subject.

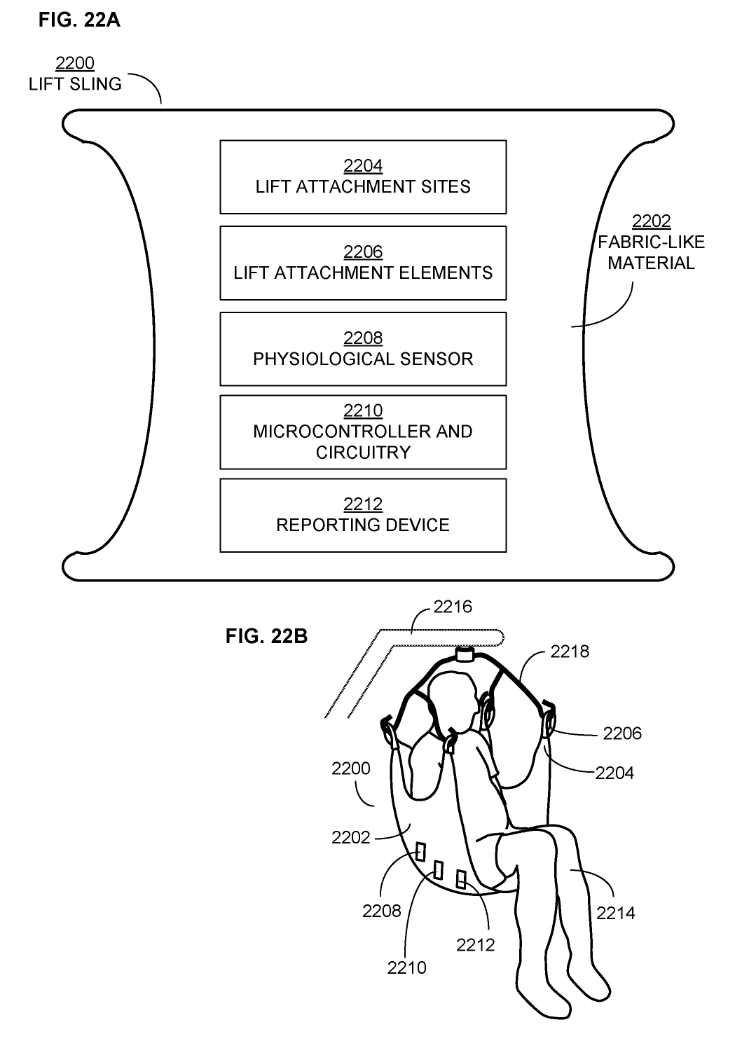

FIGS. 22A and 22B illustrate aspects of a lift sling. FIG. 22A shows a block diagram of lift sling 2200. Lift sling 2200 includes fabric-like material 2202 having a shape sufficient to at least partially cover a portion of a subject's body. In an aspect, the lift sling 2200 at least partially covers a portion of the subject's arms, legs, and torso. For example, the lift sling can include a sheet of fabric-like material that is substantially rectangular in shape and configured to cover at least a portion of the backside of a human subject. In some embodiments, the lift sling 2200 includes a type of seated sling. In some embodiments, the lift sling 2200 includes a type of supine sling. In an aspect, the fabric-like material 2202 includes a woven material. In an aspect, the fabric-like material 2202 includes a knit material. In an aspect, the fabric-like material 2202 includes a non-woven material. In an aspect, the fabric-like material 2202 is formed from at least one polymer. In an aspect, the fabric-like material 2202 is formed from polyester or nylon. In an aspect, the fabric-like material 2202 includes a plastic coated net, mesh, or webbing. For example, the fabric-like material can include a polyester mesh or solid polyester material. For example, the fabric-like material can include a Dacron mesh. In an aspect, the fabric-like material 2202 includes a non-woven polypropylene. Non-limiting aspects of fabric-like material suitable for a lift sling have been described above herein. In an aspect, the lift sling further includes padding associated with the fabric-like material to provide a supportive and comfortable sitting environment during the lift-assisted transfer.

The fabric-like material 2202 of lift sling 2200 includes one or more lift attachment sites 2204. Lift sling 2200 further includes at least one attachment element 2206 associated with the fabric-like material 2202 at at least one of the one or more lift attachment sites 2204. The at least one lift attachment element 2206 is configured to attach lift sling 2200 to a lift apparatus. In an aspect, the at least one lift attachment element 2206 includes at least one of a hook, a loop of material, or a magnet associated with the fabric-like material 2202 of lift sling 2200.

Lift sling 2200 includes one or more physiological sensors 2208 configured to measure at least one physiological parameter of the subject. Lift sling 2200 includes a microcontroller 2210 including circuitry configured to receive and process information regarding the measured at least one physiological parameter of the subject. Lift sling 2200 includes reporting device 2212 operably coupled to microcontroller 2210 and configured to transmit one or more signals indicative of the processed information regarding the measured at least one physiological parameter of the subject. In an aspect, reporting device 2212 includes at least one of an optical reporting device, an audio reporting device, a haptic reporting device, a display, or a transmission unit including an antenna. In an aspect, microcontroller 2210 includes circuitry configured to receive and process the information regarding the measured at least one physiological parameter of the subject and to determine whether the measured at least one physiological parameter of the subject falls within a range of acceptable physiological parameter values; and the reporting device 2212 operably coupled to the microcontroller 2210 is configured to transmit a control signal to the lift apparatus to control operation of the lift apparatus based on whether the measured at least one physiological parameter of the subject falls within the range of acceptable physiological parameter values. In an aspect, reporting device 2212 includes a transmission unit including an antenna configured to transmit at least one of an on/off signal, an up/down signal, a speed signal, or an acceleration signal to the lift apparatus.

FIG. 22B illustrates further aspects of lift sling 2200. Shown is lift sling 2200 holding subject 2214 and attached to an attachment portion 2218 (e.g., a spreader bar) of lift apparatus 2216. Lift sling 2200 is shown attached to attachment portion 2218 of lift apparatus 2216 with lift attachment elements 2206 associated with lift attachment sites 2204. In this non-limiting example, physiological sensor 2208, microcontroller 2210, and reporting device 2212 are shown as separate pieces associated with the fabric-like material 2202 of lift sling 2200. However, in other embodiments, physiological sensor 2208, microcontroller 2210, and/or reporting device 2212 may be combined into a signal unit.

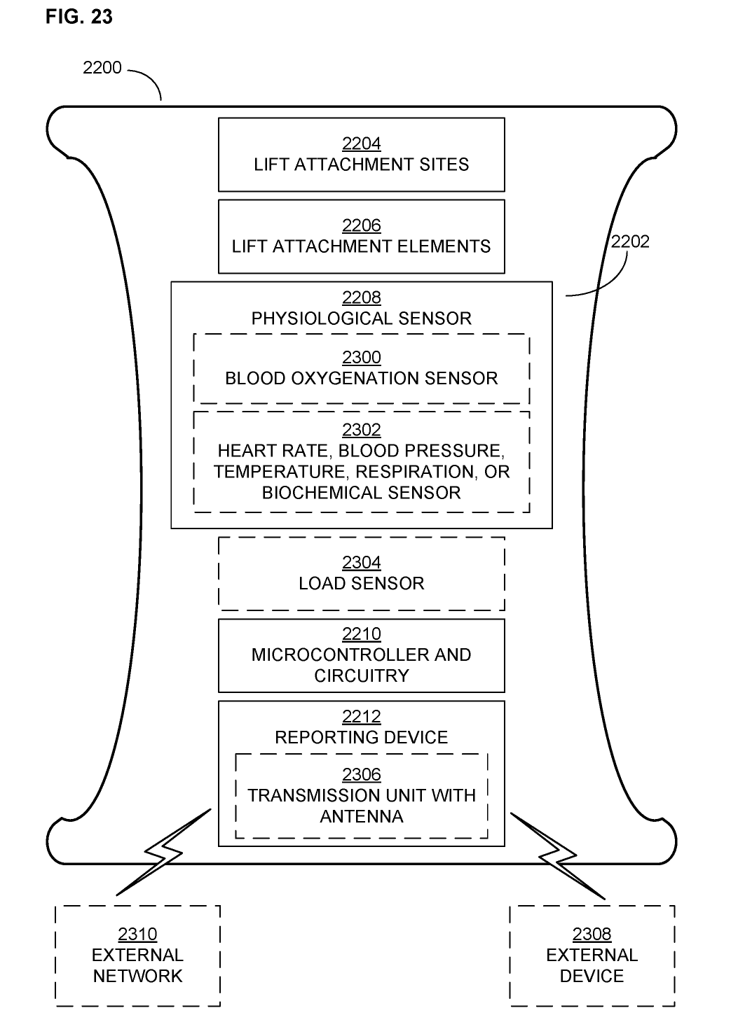

FIG. 23 illustrates further aspects of a lift sling. Lift sling 2200 includes one or more physiological sensors 2208. In some embodiments, the one or more physiological sensors 2208 include one or more blood oxygenation sensors 2300 configured to measure oxygen saturation of the subject, the one or more blood oxygenation sensors 2300 associated with at least one load path between the one or more lift attachment sites 2204. In an aspect, microcontroller 2210 includes circuitry configured to receive and process information regarding the measured oxygen saturation of the subject; and the reporting device 2212 operably coupled to the microcontroller 2210 is configured to transmit one or more signals indicative of the processed information regarding the measured oxygen saturation of the subject. In an aspect, the reporting device 2212 operably coupled to the microcontroller 2210 is configured to transmit a control signal to the lift apparatus to control operation of the lift apparatus based on the processed information regarding the measured oxygen saturation of the subject. In an aspect, reporting device 2212 operably coupled to the microcontroller 2210 is configured to transmit at least one of an on/off signal, an up/down signal, a speed signal, or an acceleration signal to the lift apparatus based on the processed information regarding the measured oxygen saturation of the subject.

In some embodiments, the one or more physiological sensors 2208 include one or more of a heart rate sensor, a blood pressure sensor, a respiration sensor, a temperature sensor, or a biochemical sensor, as shown in block 2302. In an aspect, the one or more physiological sensors 2208 are configured to measure at least one of heart rate, blood pressure, respiration, temperature, or chemistry of the subject. In an aspect, the one or more physiological sensors 2208 are configured to measure at least one physiological parameter of the subject predictive of hypoxia. Non-limiting aspects of physiological sensors are presented above herein.

In some embodiments, the lift sling further includes one or more secondary sensors configured to measure an environmental parameter experienced by the subject (e.g., air temperature, light, and/or humidity sensors) or configured to measure a positional parameter of the subject (e.g., accelerometers, gyroscopes, tilt sensors, inclination sensors, motion sensors, altimeters, and the like).

In an aspect, the reporting device 2212 includes a transmission unit 2306 including an antenna configured to communicate with an external device 2308. Non-limiting examples of external devices include the lift apparatus, a mobile communication device, or a computing device. For example, the reporting device can communicate with a smart phone to provide information regarding the measured physiological parameter of the subject. In an aspect, the reporting device 2212 includes a transmission unit 2306 including an antenna configured to communicate with an external network 2310. For example, the lift sling can include a reporting device, e.g., a transmission unit, capable of wirelessly communicating with a health provider network to enter information regarding the measured at least one physiological parameter into the subject's electronic medical record.

In some embodiments, lift sling 2200 includes a load sensor 2304 configured to measure a load. In an aspect, the load sensor 2304 is associated with at least one of the one or more lift attachment sites 2204 or along a load path between the one or more lift attachment sites 2204. In an aspect, the load sensor includes a force transducer. In an aspect, the load sensor includes at least one of a strain sensor, a stretch sensor, or a pressure sensor. Non-limiting aspects of load sensors is presented above. In an aspect, microcontroller 2210 includes circuitry configured to receive and process the information regarding the measured load; and reporting device 2212 operably coupled to microcontroller 2210 is configured to transmit one or more signals indicative of the processed information regarding the measured load. For example, the reporting device can transmit one or more of an optical signal, an audible signal, a haptic signal, or a wireless signal indicative of the processed information regarding the measured load. In an aspect, microcontroller 2210 includes circuitry configured to receive and process the information regarding the measured load and determine whether the measured load falls within a range of acceptable load values; and reporting device 2212 operably coupled to the microcontroller 2210 is configured to transmit a control signal to the lift apparatus to control at least one of an on/off operation, an up/down operation, a speed operation, or an acceleration operation of the lift apparatus based on whether the measured load falls within the range of acceptable load values.

In some embodiments, a system includes a lift sling having a shape sufficient to at least partially cover a portion of a subject's body, the lift sling including at least one lift attachment element configured to attach the lift sling to a lift apparatus; at least one blood oxygenation sensor; a microcontroller including circuitry configure to receive one or more signals from the at least one blood oxygenation sensor and configured to determine a level of hypoxia of the subject; and a transmission unit operably coupled to the microcontroller and configured to transmit one or more control signals to the lift apparatus to control an operation of the lift apparatus based on the determined level of hypoxia of the subject. In an aspect, the transmission unit is configured to transmit one or more of an on/off signal, an up/down signal, a speed signal, or an acceleration signal to the lift apparatus based on the determined level of hypoxia of the subject. In an aspect, the transmission unit is configured to wirelessly transmit a signal to the lift apparatus. In an aspect, the transmission unit is configured to transmit a signal to at least one of an external device (e.g., a mobile communication device or a computing device) or an external network (e.g., a health provider network). In an aspect, the blood oxygenation sensor, the microcontroller, and the transmission unit are attached to the lift sling. In an aspect, the blood oxygenation sensor, the microcontroller and the transmission unit are configured for attachment to the subject. For example, the blood oxygenation sensor, the microcontroller and the transmission unit can be incorporated into a wearable element, e.g., a skin patch, a wristband, or a fingertip assembly, to be worn by a subject while undergoing a lift/transfer procedure in the lift sling.

In some embodiments, a method implemented with a wearable lift device includes measuring a load value with a load sensor associated with the wearable lift device worn by a subject and attached to a lift apparatus, the wearable lift device including the load sensor; a flexible material shaped to substantially completely encircle at least a portion of the subject's body; at least one fastener configured to secure the flexible material around the at least a portion of the subject's body′ at least one lift attachment element associated with the flexible material at at least one of one or more lift attachment sites; a microcontroller including circuitry and a stored range of acceptable load values; and a reporting device operably coupled to the microcontroller; receiving and processing the measured load value with the circuitry of the microcontroller; determining whether the measured load value falls within the range of acceptable load values; and transmitting one or more control signals from the reporting device to the lift apparatus to control an operation of the lift apparatus based on whether the measured load falls within the stored range of acceptable load values.

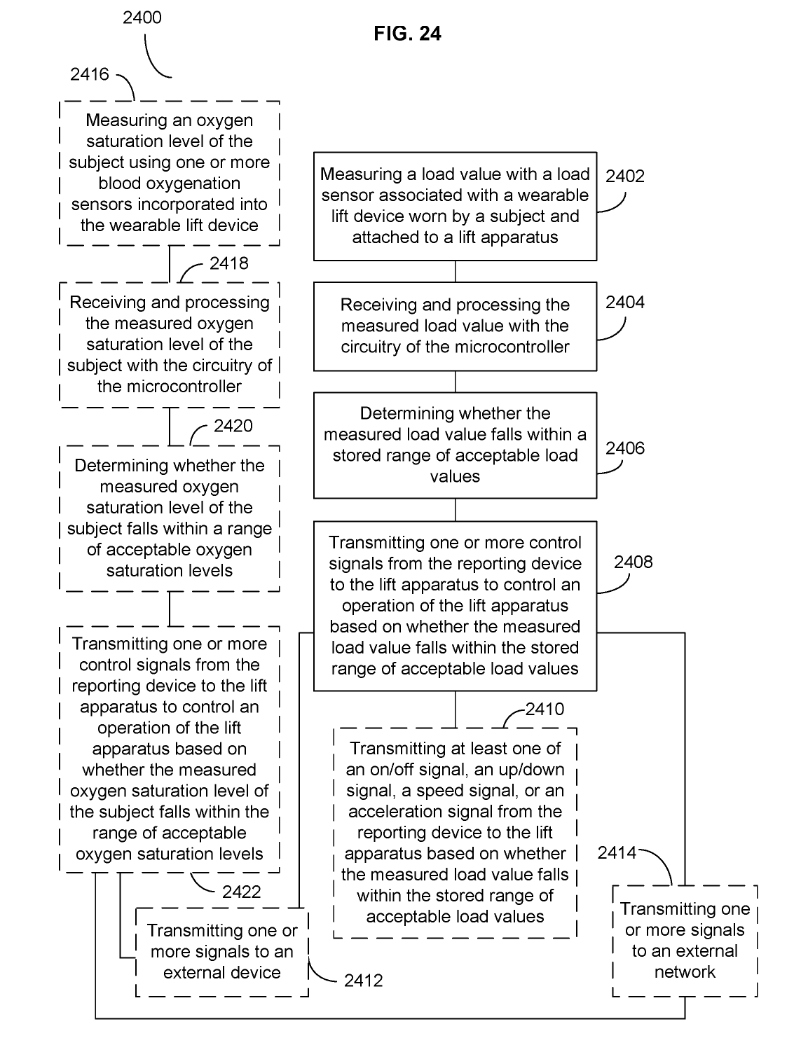

FIG. 24 illustrates aspects of a method implemented with a wearable lift device. Method 2400 includes step 2402 of measuring a load value with a load sensor associated with a wearable lift device worn by a subject and attached to a lift apparatus. In an aspect, measuring the load value with the load sensor includes measuring the load value with a load sensor associated with at least one of one or more lift attachment sites associated with the wearable lift garment or along at least one load path between the one or more lift attachment sites. In an aspect, the method includes measuring the load value with a force transducer. In an aspect, the method includes measuring the load value with at least one of a strain sensor, a stretch sensor, or a pressure sensor associated with the wearable lift garment. For example, the method can include measuring the load value along a load path between two lift attachment sites using a stretch sensor woven into a portion of the flexible material forming the wearable lift device. In an aspect, method 2400 includes comparing a measured load value from a first load sensor and a measured load value from a second load sensor; and transmitting an unlocking signal from the reporting device to the lift apparatus if the difference between the measured load value from the first load sensor and measured load value from the second load sensor falls within a range of acceptable differential load values.

Method 2400 further includes step 2404 of receiving and processing the measured load value with the circuitry of the microcontroller associated with the wearable lift device. The microcontroller is operably coupled to the load sensor and configured to receive and process information from the load sensor. Method 2400 further includes step 2406 of determining whether the measured load value falls within a stored range of acceptable load values. In an aspect, the microcontroller of the wearable lift device compares the measured load value with a stored range of acceptable load values stored in a memory component of the microcontroller.

Method 2400 further includes step 2408 of transmitting one or more control signals from the reporting device associated with the wearable lift device to the lift apparatus to control an operation of the lift apparatus based on whether the measured load value falls within the stored range of acceptable load values. For example, the method can include transmitting a control signal from the reporting device to the lift apparatus to slow down the speed of the lift apparatus if the measured load value fails to fall within the stored range of acceptable load values. In an aspect, method 2400 includes in step 2410 transmitting at least one of an on/off signal, an up/down signal, a speed signal, or an acceleration signal from the reporting device to the lift apparatus based on whether the measured load value falls within the stored range of acceptable load values.

In an aspect, the method includes transmitting at least one of an optical signal, an audible signal, or a haptic signal from the reporting device of the wearable lift device. In an aspect, the method includes transmitting one or more wireless signals from the reporting device of the wearable lift apparatus. In an aspect, the reporting device includes an optical reporting device, an audio reporting device, a haptic reporting device, a display, and/or a transmission unit.

In an aspect, method 2400 includes step 2412 of transmitting one or more signals from the reporting device to an external device. For example, the method can include transmitting one or more signals from a transmission unit associated with the wearable lift device to an external device. In an aspect, the method includes transmitting the one or more signals from the reporting device of the wearable lift garment to at least one of a mobile communication device or a computing device. For example, the method can include transmitting the one or more signals indicative of the measured load value to a smart phone. For example, the method can include transmitting one or more signals indicative of the measured load value to a tablet, laptop, or desktop computing device. In an aspect, the method includes transmitting the one or more signals to an external device located in the same room with the subject wearing the wearable lift device. For example, the method can include transmitting the one or more signals to a mobile communication device or a computing device located in a hospital, medical clinic, skilled nursing, or assisted living facility in which the subject is located. For example, the method can include transmitting the one or more signals to a mobile communication device or a computing device located in a residence in which the subject is located. In an aspect, the method includes transmitting the one or more signals to an external device located in a remote location relative to the location of the subject wearing the wearable lift device. For example, the method can include transmitting the one or more signals to a remote mobile communication device or a computing device associated with a physician or other healthcare provider.

In an aspect, method 2400 includes step 2414 of transmitting the one or more signals from the reporting device to an external network. For example, the method can include transmitting one or more signals from the reporting device of the wearable lift device to a health provider network. For example, the method can include wirelessly transmitting the one or more signals through an antenna of a transmission unit associated with the wearable lift device to a network associated with a hospital, medical clinic, skilled nursing facility, or assisted living facility. For example, the method can include wirelessly transmitting the one or more signals from the reporting device of the wearable lift device to the subject's medical record stored in a health provider network.

Method 2400 further includes securing the wearable lift device around at least a portion of the subject's body with at least one fastener; and attaching the wearable lift device to the lift apparatus with at least one lift attachment element. The method can include securing the wearable lift device around the at least a portion of the subject's body with at least one of a buckle, a cinch, a hook and loop fastener, a hook and eye fastener, a snap, or a zipper. The method can include attaching the wearable lift device to the lift apparatus with at least one of a hook, a loop of material, or a magnet associated with the wearable lift device.

In some embodiments, method 2400 includes measuring at least one physiological parameter of the subject with one or more physiological sensors incorporated into the wearable lift device; receiving and processing the measured at least one physiological parameter of the subject with the circuitry of the microcontroller; determining whether the measured at least one physiological parameter of the subject falls within a range of acceptable physiological parameter values; and transmitting one or more signals from the reporting device indicative whether the measured at least one physiological parameter of the subject falls within the range of acceptable physiological parameter values. In some embodiments, the method includes measuring the at least one physiological parameter of the subject with at least one of a heart rate sensor, a blood pressure sensor, a respiratory sensor, or a biochemical sensor associated with the wearable lift device. In an aspect, method 2400 further includes transmitting a control signal from the reporting device to the lift apparatus to control at least one of an on/off, an up/down function, a speed function, or an acceleration function of the lift apparatus in response to the measured at least one physiological parameter of the subject. In an aspect, method 2400 includes transmitting one or more signals to at least one of an external device or an external network in response to the measured at least one physiological parameter of the subject.

In some embodiments, the method includes measuring the oxygen saturation of the subject while wearing the wearable lift device. In an aspect, method 2400 includes step 2416 of measuring an oxygen saturation level of the subject using one or more blood oxygenation sensors incorporated into the wearable lift device; step 2418 of receiving and processing the measured oxygen saturation level of the subject with the circuitry of the microcontroller; step 2420 of determining whether the measured oxygen saturation level of the subject falls within a range of acceptable oxygen saturation levels; and step 2422 of transmitting one or more control signals from the reporting device to the lift apparatus to control an operation of the lift apparatus based on whether the measured oxygen saturation level of the subject falls within the range of acceptable oxygen saturation levels. In an aspect, method 2400 includes measuring the oxygen saturation level of the subject with a near infrared blood oxygenation sensor. In an aspect, method 2400 includes transmitting one or more signals to at least one of an external device or an external network in response to the measured oxygen saturation level of the subject. For example, the method can include transmitting information regarding the measured oxygen saturation level of the subject to a mobile communication device, e.g., a smart phone. For example, the method can include transmitting information regarding the measured oxygen saturation level of the subject to a health provider network.

In some embodiments, a method implemented with a wearable lift device includes measuring at least one physiological parameter of a subject with one or more physiological sensors associated with the wearable lift device worn by the subject and attached to a lift apparatus, the wearable lift device including the one or more physiological sensors, a flexible material shaped to substantially completely encircle at least a portion of the subject's body, at least one fastener configured to secure the flexible material around the at least a portion of the subject's body, at least one lift attachment element associated with the flexible material at at least one of one or more lift attachment sites, a microcontroller including circuitry and a stored range of acceptable physiological parameter values, and a reporting device operably coupled to the microcontroller; receiving and processing the measured at least one physiological parameter of the subject with the circuitry of the microcontroller; determining whether the measured at least one physiological parameter of the subject falls within the stored range of acceptable physiological parameter values; and transmitting one or more control signals from the reporting device to the lift apparatus to control an operation of the lift apparatus based on whether the measured at least one physiological parameter of the subject falls within the stored range of acceptable physiological parameter values.

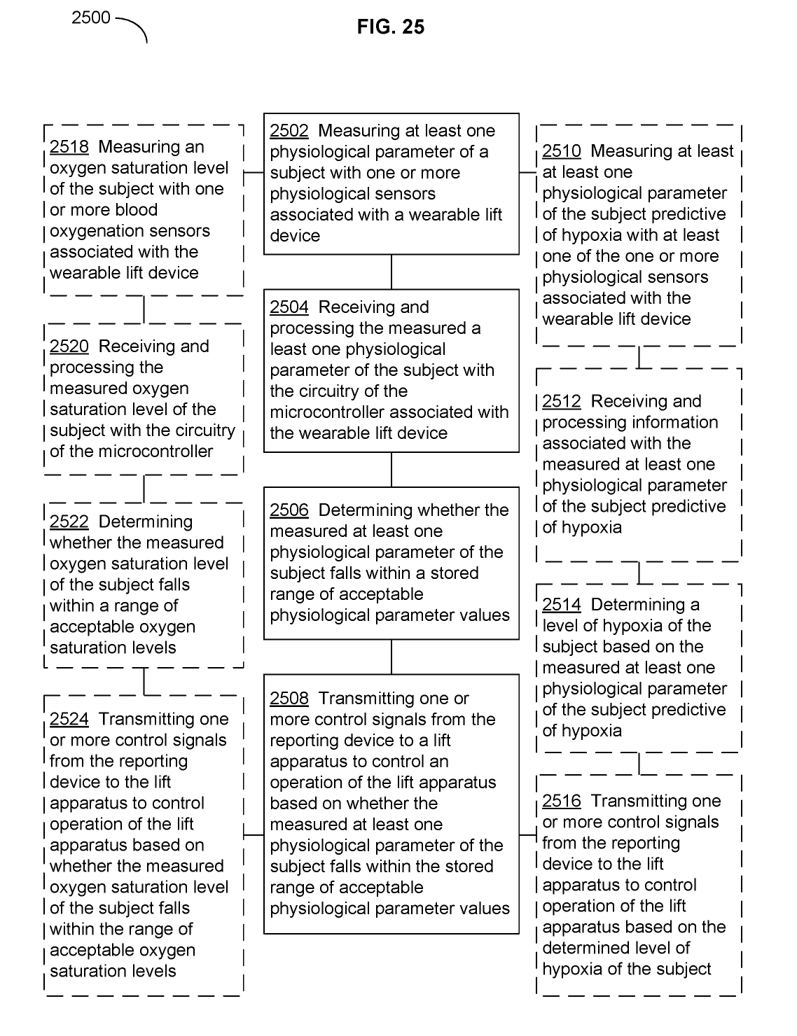

FIG. 25 illustrates aspects of a method implemented with a wearable lift device. Method 2500 includes in step 2502 measuring at least one physiological parameter of a subject with one or more physiological sensors associated with a wearable lift device. The method can include measuring the at least one physiological parameter of the subject with at least one of a heart rate sensor, a blood pressure sensor, a respiration sensor, a body temperature sensor, or a biochemical sensor. The method can include measuring at least one of heart rate, blood pressure, respiration, body temperature, or biochemical property of the subject.

Method 2500 further includes in step 2504 receiving and processing the measured at least one physiological parameter of the subject with the circuitry of the microcontroller associated with the wearable lift device. The method includes receiving and processing information from the one or more physiological sensors regarding the measured at least one physiological parameter of the subject. Method 2500 further includes in step 2506 determining whether the measured at least one physiological parameter of the subject falls within a stored range of acceptable physiological parameter values; and in step 2508 transmitting one or more control signals from the reporting device to a lift apparatus to control an operation of the lift apparatus based on whether the measured at least one physiological parameter of the subject falls within the stored range of acceptable physiological parameter values. In an aspect, method 2500 includes transmitting at least one of an on/off signal, an up/down signal, a speed signal, or an acceleration signal from the reporting device to the lift apparatus based on whether the measured at least one physiological parameter of the subject falls within the stored range of acceptable physiological parameter values.

In an aspect, method 2500 further includes transmitting one or more of an optical signal, an audible signal, a haptic signal, or a wireless signal from the reporting device of the wearable lift device indicative of whether the measured at least one physiological parameter of the subject falls within the stored range of acceptable physiological parameter values. In an aspect, the reporting device includes an optical reporting device, an audio reporting device, a haptic reporting device, a display, and/or a transmission unit. In an aspect, method 2500 includes transmitting one or more signals from the reporting device of the wearable lift device to an external device. For example, the method can include transmitting one or more signals indicative of the measured at least one physiological parameter of the subject to a mobile communication device and/or a computing device. In an aspect, method 2500 includes transmitting one or more signals from the reporting device of the wearable lift device to an external network. For example, the method can include transmitting one or more signals indicative of the measured at least one physiological parameter of the subject to a health provider network.

In some embodiments, method 2500 implemented with a wearable lift device includes in step 2510 measuring at least one physiological parameter of the subject predictive of hypoxia with at least one of the one or more physiological sensors associated with the wearable lift device; in step 2512 receiving and processing information associated with the measured at least one physiological parameter of the subject predictive of hypoxia; in step 2514 determining a level of hypoxia of the subject based on the measured at least one physiological parameter of the subject predictive of hypoxia, and in step 2516 transmitting one or more control signals from the reporting device to the lift apparatus to control operation of the lift apparatus based on the determined level of hypoxia of the subject.

In some embodiments, method 2500 implemented with a wearable lift device includes in step 2518 measuring an oxygen saturation level of the subject with one or more blood oxygenation sensors associated with the wearable lift device; in step 2520 receiving and processing the measured oxygen saturation level of the subject with the circuitry of the microcontroller; in step 2522 determining whether the measured oxygen saturation level of the subject falls within a range of acceptable oxygen saturation levels; and in step 2524 transmitting one or more control signals from the reporting device to the lift apparatus to control an operation of the lift apparatus based on whether the measured oxygen saturation level of the subject falls within the range of acceptable oxygen saturation levels.

In some embodiments, a method implemented with a lift sling includes measuring an oxygen saturation level of a subject with at least one blood oxygenation sensor associated with the lift sling attached to a lift apparatus, the lift sling including the at least one blood oxygenation sensor, a flexible material having a shape sufficient to at least partially encircle a portion of the subject's body, at least one lift attachment element associated with the flexible material at at least one of one or more lift attachment sites, a microcontroller including circuitry and a stored range of acceptable oxygen saturation levels, and a reporting device operably coupled to the microcontroller; receiving and processing the measured oxygen saturation level of the subject with the circuitry of the microcontroller, determining whether the measured oxygen saturation level of the subject falls within the stored range of acceptable oxygen saturation levels; and transmitting one or more signals with the reporting device indicative of whether the measured oxygen saturation level of the subject falls within the stored range of acceptable oxygen saturation levels.

In an aspect, the method includes measuring the oxygen saturation level of the subject with a near infrared optical blood oxygenation sensor associated with lift sling. In an aspect, the method includes measuring the oxygen saturation with at least one blood oxygenation sensor associated with at least one load path between the one or more lift attachment sites. In an aspect, the method includes measuring the oxygen saturation level of the subject with two or more blood oxygenation sensors distributed along a length of the at least one load path between the one or more lift attachment sites. For example, the method can include using one or more blood oxygenation sensors to determine whether the process of sitting in a lift sling and/or being moved in a lift sling is limiting blood circulation or otherwise causing the subject to become hypoxic.

The method includes transmitting one or more signals with a reporting device associated with lift sling indicative of the measured oxygen saturation level of the subject. In an aspect, the method includes transmitting one or more of an optical signal, an audible signal, a haptic signal, or a wireless signal indicative of the measured oxygen saturation level of the subject. In an aspect, the method includes transmitting one or more signals from at least one of an optical reporting device, an audio reporting device, a haptic reporting device, a display, or a transmission unit associated with the lift sling. In an aspect, the method includes wirelessly transmitting the one or more signals from the reporting device to an external device, e.g., a mobile communication device or a computing device. In an aspect, the method includes wirelessly transmitting one or more signals from the reporting device to an external network, e.g., a health provider network. In an aspect, the method includes transmitting one or more control signals from the reporting device to a lift apparatus to control a function of the lift apparatus, wherein the function of the lift apparatus includes at least one of an on/off function, an up/down function, an acceleration function, or a speed function. In an aspect, the method includes transmitting a locking signal from the reporting device to the lift apparatus. For example, the method can include wirelessly transmitting a locking signal from the reporting device of the lift sling to the lift apparatus if the measured oxygen saturation level of the subject fails to fall within the range of acceptable oxygen saturation levels. In an aspect, the method includes transmitting an unlocking signal from the reporting device to the lift apparatus. For example, the method can include wirelessly transmitting an unlocking signal from the reporting device of the lift sling to the lift apparatus if the measured oxygen saturation level of the subject falls within the range of acceptable oxygen saturation levels.

In an aspect, a method implemented with a microcontroller includes receiving one or more signals indicative of a measured oxygen saturation of a subject from at least one blood oxygenation sensor; determining a level of hypoxia of the subject by comparing the measured oxygen saturation of the subject with a range of acceptable oxygen saturation levels stored in the microcontroller; and transmitting one or more control signals from a transmission unit operably coupled to the microcontroller to a lift apparatus to control operation of the lift apparatus based on the determined level of hypoxia of the subject. In an aspect, the method includes transmitting at least one of an on/off signal, an up/down signal, a speed signal, or an acceleration signal from the transmission unit operably coupled to the microcontroller to a lift apparatus to control the operation of the lift apparatus based on the determined level of hypoxia of the subject.

The state of the art has progressed to the point where there is little distinction left between hardware, software, and/or firmware implementations of aspects of systems; the use of hardware, software, and/or firmware is generally (but not always, in that in certain contexts the choice between hardware and software can become significant) a design choice representing cost vs. efficiency tradeoffs. There are various vehicles by which processes and/or systems and/or other technologies described herein can be effected (e.g., hardware, software, and/or firmware), and that the preferred vehicle will vary with the context in which the processes and/or systems and/or other technologies are deployed. For example, if an implementer determines that speed and accuracy are paramount, the implementer may opt for a mainly hardware and/or firmware vehicle; alternatively, if flexibility is paramount, the implementer may opt for a mainly software implementation; or, yet again alternatively, the implementer may opt for some combination of hardware, software, and/or firmware. Hence, there are several possible vehicles by which the processes and/or devices and/or other technologies described herein can be effected, none of which is inherently superior to the other in that any vehicle to be utilized is a choice dependent upon the context in which the vehicle will be deployed and the specific concerns (e.g., speed, flexibility, or predictability) of the implementer, any of which may vary. Those skilled in the art will recognize that optical aspects of implementations will typically employ optically-oriented hardware, software, and or firmware.

In some implementations described herein, logic and similar implementations can include software or other control structures. Electronic circuitry, for example, may have one or more paths of electrical current constructed and arranged to implement various functions as described herein. In some implementations, one or more media can be configured to bear a device-detectable implementation when such media hold or transmit device detectable instructions operable to perform as described herein. In some variants, for example, implementations can include an update or modification of existing software or firmware, or of gate arrays or programmable hardware, such as by performing a reception of or a transmission of one or more instructions in relation to one or more operations described herein. Alternatively or additionally, in some variants, an implementation can include special-purpose hardware, software, firmware components, and/or general-purpose components executing or otherwise invoking special-purpose components. Specifications or other implementations can be transmitted by one or more instances of tangible transmission media as described herein, optionally by packet transmission or otherwise by passing through distributed media at various times.

Alternatively or additionally, implementations may include executing a special-purpose instruction sequence or otherwise invoking circuitry for enabling, triggering, coordinating, requesting, or otherwise causing one or more occurrences of any functional operations described above. In some variants, operational or other logical descriptions herein may be expressed directly as source code and compiled or otherwise invoked as an executable instruction sequence. In some contexts, for example, C++ or other code sequences can be compiled directly or otherwise implemented in high-level descriptor languages (e.g., a logic-synthesizable language, a hardware description language, a hardware design simulation, and/or other such similar mode(s) of expression). Alternatively or additionally, some or all of the logical expression may be manifested as a Verilog-type hardware description or other circuitry model before physical implementation in hardware, especially for basic operations or timing-critical applications. Those skilled in the art will recognize how to obtain, configure, and optimize suitable transmission or computational elements, material supplies, actuators, or other common structures in light of these teachings.

The foregoing detailed description has set forth various embodiments of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, or examples can be implemented, individually and/or collectively, by a wide range of hardware, software, firmware, or virtually any combination thereof. In one embodiment, several portions of the subject matter described herein can be implemented via Application Specific Integrated Circuits (ASICs), Field Programmable Gate Arrays (FPGAs), digital signal processors (DSPs), or other integrated formats. However, some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. In addition, the mechanisms of the subject matter described herein are capable of being distributed as a program product in a variety of forms, and that an illustrative embodiment of the subject matter described herein applies regardless of the particular type of signal bearing medium used to actually carry out the distribution.

In a general sense, those skilled in the art will recognize that the various embodiments described herein can be implemented, individually and/or collectively, by various types of electro-mechanical systems having a wide range of electrical components such as hardware, software, firmware, and/or virtually any combination thereof and a wide range of components that may impart mechanical force or motion such as rigid bodies, spring or torsional bodies, hydraulics, electro-magnetically actuated devices, and/or virtually any combination thereof. Consequently, as used herein “electro-mechanical system” includes, but is not limited to, electrical circuitry operably coupled with a transducer (e.g., an actuator, a motor, a piezoelectric crystal, a Micro Electro Mechanical System (MEMS), etc.), electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of memory (e.g., random access, flash, read only, etc.)), electrical circuitry forming a communications device (e.g., a modem, communications switch, optical-electrical equipment, etc.), and/or any non-electrical analog thereto, such as optical or other analogs. Those skilled in the art will also appreciate that examples of electro-mechanical systems include but are not limited to a variety of consumer electronics systems, medical devices, as well as other systems such as motorized transport systems, factory automation systems, security systems, and/or communication/computing systems. Those skilled in the art will recognize that electro-mechanical as used herein is not necessarily limited to a system that has both electrical and mechanical actuation except as context may dictate otherwise.

In a general sense, the various aspects described herein can be implemented, individually and/or collectively, by a wide range of hardware, software, firmware, and/or any combination thereof and can be viewed as being composed of various types of “electrical circuitry.” Consequently, as used herein “electrical circuitry” includes, but is not limited to, electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a microprocessor configured by a computer program which at least partially carries out processes and/or devices described herein), electrical circuitry forming a memory device (e.g., forms of memory (e.g., random access, flash, read only, etc.)), and/or electrical circuitry forming a communications device (e.g., a transmission unit, communications switch, optical-electrical equipment, etc.). The subject matter described herein can be implemented in an analog or digital fashion or some combination thereof.

Those skilled in the art will recognize that at least a portion of the systems and/or processes described herein can be integrated into a data processing system. A data processing system generally includes one or more of a system unit housing, a video display device, memory such as volatile or non-volatile memory, processors such as microprocessors or digital signal processors, computational entities such as operating systems, drivers, graphical user interfaces, and applications programs, one or more interaction devices (e.g., a touch pad, a touch screen, an antenna, etc.), and/or control systems including feedback loops and control motors (e.g., feedback for sensing position and/or velocity; control motors for moving and/or adjusting components and/or quantities). A data processing system can be implemented utilizing suitable commercially available components, such as those typically found in data computing/communication and/or network computing/communication systems.

Those skilled in the art will recognize that at least a portion of the systems and/or processes described herein can be integrated into a mote system. Those having skill in the art will recognize that a typical mote system generally includes one or more memories such as volatile or non-volatile memories, processors such as microprocessors or digital signal processors, computational entities such as operating systems, user interfaces, drivers, sensors, actuators, applications programs, one or more interaction devices (e.g., an antenna USB ports, acoustic ports, etc.), control systems including feedback loops and control motors (e.g., feedback for sensing or estimating position and/or velocity; control motors for moving and/or adjusting components and/or quantities). A mote system may be implemented utilizing suitable components, such as those found in mote computing/communication systems. Specific examples of such components entail such as Intel Corporation's and/or Crossbow Corporation's mote components and supporting hardware, software, and/or firmware.

One skilled in the art will recognize that the herein described components (e.g., operations), devices, objects, and the discussion accompanying them are used as examples for the sake of conceptual clarity and that various configuration modifications are contemplated. Consequently, as used herein, the specific exemplars set forth and the accompanying discussion are intended to be representative of their more general classes. In general, use of any specific exemplar is intended to be representative of its class, and the non-inclusion of specific components (e.g., operations), devices, and objects should not be taken limiting.

With respect to the use of substantially any plural and/or singular terms herein, those having skill in the art can translate from the plural to the singular and/or from the singular to the plural as is appropriate to the context and/or application. The various singular/plural permutations are not expressly set forth herein for sake of clarity.

The herein described subject matter sometimes illustrates different components contained within, or connected with, different other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “operably coupled to” each other such that the desired functionality is achieved, irrespective of architectures or intermedial components. Likewise, any two components so associated can also be viewed as being “operably connected,” or “operably coupled,” to each other to achieve the desired functionality, and any two components capable of being so associated can also be viewed as being “operably couplable,” to each other to achieve the desired functionality. Specific examples of operably couplable include but are not limited to physically mateable and/or physically interacting components, and/or wirelessly interactable, and/or wirelessly interacting components, and/or logically interacting, and/or logically interactable components.

In some instances, one or more components can be referred to herein as “configured to,” “configured by,” “configurable to,” “operable/operative to,” “adapted/adaptable,” “able to,” “conformable/conformed to,” etc. Those skilled in the art will recognize that such terms (e.g. “configured to”) can generally encompass active-state components and/or inactive-state components and/or standby-state components, unless context requires otherwise.

While particular aspects of the present subject matter described herein have been shown and described, it will be apparent to those skilled in the art that, based upon the teachings herein, changes and modifications can be made without departing from the subject matter described herein and its broader aspects and, therefore, the appended claims are to encompass within their scope all such changes and modifications as are within the true spirit and scope of the subject matter described herein.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that typically a disjunctive word and/or phrase presenting two or more alternative terms, whether in the description, claims, or drawings, should be understood to contemplate the possibilities of including one of the terms, either of the terms, or both terms unless context dictates otherwise. For example, the phrase “A or B” will be typically understood to include the possibilities of “A” or “B” or “A and B.”

Various non-limiting embodiments are described herein as Prophetic Examples.

### Prophetic Example 1: A Wearable Lift Garment with Load Sensors and Microcircuitry to Safely Move Patients Using a Lift Apparatus

A wearable lift garment is constructed from a strong textile material that includes lift attachment sites for connection to a mobile lift apparatus and sensors and reporters to increase safety for the patient and the caregiver. A lift garment that is worn by the patient facilitates movement of the patient whenever necessary. For example, a one piece undergarment with short sleeves and shorts may be constructed with lift attachment sites, load sensors and microcircuitry that promote safe attachment and movement with a patient lift. See FIGS. 1 and 2. A woven fabric with ultra-high molecular weight polyethylene (UHMwPE) yarn blended with nylon or polyester may be designed to have very high tensile strength and to resist tears. Fabrics woven with spun yarns of UHMwPE (e.g., Dyneema®) are available from DSM Dyneema LLC, Stanley, N.C. For example, high performance, super-strength, load bearing Dyneema® fabrics are described (see e.g., Dyneema® products online: http://www.dsm.com/products/dyneema/en GB/sports-lifestyle/dyneema-in-sports-equipment.html). The lift garment may be cut from woven fabric with containing approximately 20% UHMwPE and 80% polyester yarns, and then sewn with UHMwPE webbing reinforcing the seams. Fabric loops of UHMwPE for attachment to a lift apparatus are incorporated in the garment at strategic lift attachment sites (see, e.g., FIG. 2A). The attachment loops connect with textile load sensors that are incorporated in the garment.

The lift garment incorporates load sensors at the lift attachment sites to measure the force applied to the lift garment and the lift during movement of a patient. Information from the load sensors is processed and compared to predetermined load limits for the lift apparatus. If load limits are exceeded, signals are transmitted to alert the caregiver, lock the lift apparatus and protect the patient. Textile load sensors connect the attachment loops to the lift garment and measure the strain and stress at the lift attachment site when the patient is suspended. Textile load sensors are woven from conductive fibers and nonconductive fibers to create a strain gauge which responds to tensile load and strain, i.e., deformation, with a change in resistance. For example, a textile strain gauge can be created with a nonconducting fiber, e.g., Lycra fiber, and a conducting fiber, e.g., carbon coated polyamide fiber (see e.g., Shyr et al., Sensors 11: 1693-1705, 2011 which is incorporated herein by reference). Multiple textile load sensors may be incorporated on a load path between two lift attachment sites. Moreover, additional load sensors may be incorporated on additional load paths to monitor the forces applied to the lift apparatus. The multiple load sensors may be connected in parallel to the microcircuitry in the lift garment to yield an average signal characterizing the weight (load) on the lift garment and the lift apparatus.

The lift garment is constructed with electronic components to receive, process and transmit electronic signals from its sensors. Flexible electronics components to process signals from the garment's textile sensors can be complementary metal-oxide-semiconductor (CMOS) integrated circuits, arrays of transistors, inverters, oscillators, and amplifiers. Materials and designs for flexible electronics with linear elastic responses are described (see e.g., Kim et al., Proc. Natl. Acad. Sci., USA 105: 18675-18680, 2008 which is incorporated herein by reference). For example, integrated circuitry embedded in poly(dimethylsiloxane) (PDMS) is attached to or interwoven in the fabric of the lift garment. Conducting fibers from the load sensors connect to the integrated circuitry of the microcontroller. Interconnections between sensors, power supplies and microcircuitry can be made with conductive adhesive (see e.g., Stoppa et al., Sensors 14: 11957-11992, 2014 which is incorporated herein by reference). A power source is also incorporated in the fabric of the lift garment and connected to empower the electronic components and microcircuitry. For example, a battery that is fabricated by screen printing of silver-oxide onto textile substrates is described (see e.g., Stoppa et al., Ibid.).

The lift garment microcontroller analyzes load data obtained from the load sensors when the patient is suspended in the lift apparatus. If the patient weight exceeds safe limits for the lift apparatus and/or the caregiver, or the load force exceeds limits along any load path the reporting device sends a signal to alert the caregiver and lock the lift apparatus in order to prevent further movement of the patient. Electronic signals indicating load forces at each lift attachment site and each load path are displayed on digital displays incorporated in the lift garment at each attachment site to alert the caregiver. Moreover the digital displays report a color code: red for excessive load force and green for acceptable load force. Calculated load forces are transmitted wirelessly to the lift apparatus via a textile antenna incorporated in the lift garment. For example, a textile planar antenna approximately 50 mm×46 mm with a bandwidth of approximately 180 MHz can be incorporated in the lift garment and connected to the microcontroller. Flexible textile antennas are described (see e.g., Hertleer et al., IEEE Transactions on Antennas and Propagation, 57: 919-925, 2009 which is incorporated herein by reference). Wireless signals transmitted via the lift garment antenna are received by the lift apparatus where a controller controls the lift motor power supply. If wireless signals from the lift garment microcontroller indicate excessive load forces are present on the lift apparatus then the lift apparatus controller shuts off power to the lift motor and locks the lift apparatus in place. Smart fabric touchpad controls incorporated in the lift garment allow unlocking the lift and lowering the patient to a safe location on a bed or chair. Smart fabric touchpad controls (available from Wearable Technologies Limited, London) can be used to signal the lift apparatus and safely raise or lower the patient. A fabric touchpad that signals to microcircuitry is described (see e.g., Our Technologies Page at http://www.wearable.technology/index.php/our-technologies which is incorporated herein by reference).

The lift garment also monitors physiologic parameters of the patient and reports them to a health provider network (e.g., hospital, nursing facility or personal caregiver in the home). The lift garment incorporates textile sensors to monitor basic physiologic parameters including: heart rate, respiration, and temperature. Sensors comprised of piezoelectric fibers are incorporated in the lift garment opposite the chest and abdomen of the patient. Knitted piezoresistive fabric sensors to monitor respiration are described (see e.g., Pacelli, et al., Proceedings of the 3rd IEEE-EMBS, International Summer School and Symposium on Medical Devices and Biosensors, MIT, Boston, Mass., 2006 which is incorporated herein by reference). Also fabric electrodes constructed from stainless steel threads and a nonconducting fiber can be integrated in the lift garment in the chest and abdomen areas to detect electrocardiograhic signals (see e.g., Pacelli, et al., Ibid.). Flexible temperature sensors based on composite fibers are incorporated in the lift garment to monitor body temperature in the patient. Temperature sensor signals are sent to the microcontroller and temperature data are reported to a local or remote health provider network. For example, a temperature sensor constructed from composite fibers can be incorporated in the fabric of the lift garment and operate in the 30-42° C. temperature range (see e.g., Sibinski et al., Sensors 10: 7934-7946, 2010 which is incorporated herein by reference). Physiologic data on heart rate, respiration and temperature is analyzed by the lift garment microcontroller and the patient's physiologic parameters are compared to baseline values for the patient and to age- and condition-adjusted normal ranges for the patient. Physiologic parameters outside the normal range activate the reporting device, e.g., a transmission unit, to signal the lift apparatus to lock the lift apparatus. For example, if electrocardiograph signals become aberrant during lifting of the patient the reporting device can signal the lift apparatus to stop lifting, lock the apparatus and slowly lower the patient to their bed. Temporal variation in physiologic parameters can also activate safety protocols in the lift garment microcontroller. For example, if a patient's temperature has risen sharply during the lift procedure the lift garment can alert the caregiver using color coded digital display and send a signal to the lift apparatus to abort the lift procedure. Aborting the lift procedure can include reversing the direction of the lift apparatus (ie. lowering/raising), or locking the lift apparatus and moving the patient, or combinations of these maneuvers to return the patient to their bed or an emergency room or an ambulance.

### Prophetic Example 2: A Wearable Lift Device with Sensors and Micro Circuitry to Safely Control Patient Lift Apparatus and Report Physiological Parameters to Medical Personnel

A wearable lift device is constructed with sensors, micro circuitry, webbing, fasteners and an attachment ring to facilitate moving a patient with a lift apparatus and to safeguard the patient and their caregiver. Webbing straps comprised of polypropylene are designed to surround the patient and attach to a lift apparatus, a non-limiting example of which is shown in FIGS. 17A and 17B. The webbing straps are securely fastened around the patient with adjustable cam buckles. For example, metal cam buckles with a breaking strength of approximately 1200 lbs. can be attached to polypropylene webbing straps also rated at 1200 lbs. (metal cam buckles and polypropylene webbing are available from Strapworks.com, Eugene, Oreg.). To engage a lift apparatus the wearable lift device has an attachment site (see FIG. 17B, #1604) that includes a metal ring which accepts a load bearing cable (see FIG. 17B, #1708) from the lift apparatus. The attachment ring is connected to the webbing straps via loops sewn into the webbing. The attachment ring also accepts a tension load cell. For example, a miniature tension load cell with a capacity of 1000 lbs. and an output of 2 mV/V is incorporated to measure the load forces on the lift apparatus (see e.g., Load Cell Spec Sheet available from Omega Engineering Inc., Stamford, Conn. which is incorporated herein by reference). Output from the load cell is sent via micro circuitry to a microcontroller constructed in the wearable lift device. In addition, electronic-textile load sensors are incorporated in the polypropylene straps to detect load forces along load paths emanating from the attachment ring. A textile load sensor (i.e., strain gauge) can be created with a nonconducting fiber, e.g., Lycra fiber, and a conducting fiber, e.g., carbon coated polyamide fiber (see e.g., Shyr et al., Ibid.). Load forces detected on individual load paths are sent to the microcontroller and compared to look for unsafe weight distribution as well as safe limits of total weight. A display with integrated light emitting diodes (LEDs) is integrated into a portion of the webbing to indicate the load forces detected by the load sensors. Displays are positioned adjacent to the load sensors they report and provide information on the distribution of load forces as well as the total weight detected. The flexible displays are addressable and can display numerical values for load forces and color indicators for safe or unsafe loads. Fabric displays comprised of LEDs and connected with fabric fiber conductors are described (see e.g., U.S. Pat. No. 7,144,830 issued to Hill et al. on Dec. 5, 2006 which is incorporated herein by reference).

Microcircuitry and microprocessors incorporated in the webbing of the wearable lift device analyze the load forces detected by the miniature load cell and the textile load sensors. Microcircuitry and microprocessors integrated into textiles have been described. For example, electronic circuitry and electronic components can be created on textiles by printing conductive materials (e.g., silver inks or conductive polymers) onto a fabric composed of polyethylene and nylon fibers (see e.g., Karaguzel et al., Journal of The Textile Institute 100: 1-9, 2009 and U.S. Pat. No. 8,752,285 issued to Son et al. on Jun. 17, 2014 which are incorporated herein by reference). Flexible fabric electronic circuitry displays conductivity comparable to copper foil traces used on printed circuit boards. Silver ink traces printed on polyester/nylon fabric show approximately 42 times higher resistance relative to equal-sized traces of copper foil on a standard printed circuit board (see e.g., Karaguzel et al., Ibid.), and are suitable for electronic textiles.

The wearable lift device is constructed with physiological sensors to monitor the patient's well-being before, during and after the lifting procedure. Physiological sensors are integrated in the wearable lift device and signal to a microcontroller which transmits the health data to a health provider network and the patient's caregiver. Physiological sensors to monitor blood oxygenation (SpO₂), electrocardiography (ECG), and respiration are incorporated in the lift garment. An oxygenation sensor is embedded in the wearable lift device to overlay the sternum. The SpO₂ sensor includes red and infrared LEDs that emit light to the sternum, and an array of optical fibers that detect reflected light. Optical fibers incorporated in the webbing of the wearable lift device transmit reflected light to a photodetector and microcontroller for analysis. Fabric-embedded blood oxygen sensors are described (see e.g., Coyle et al., IEEE Trans. Inf. Technol. Biomed. 14: 364-370, 2010 which is incorporated herein by reference). Data on blood oxygenation, i.e., percent SpO₂, is transmitted to a health provider network (e.g., hospital information system) and to the patient's caregiver (e.g., cell phone or tablet). Also if SpO₂ values are outside the normal range indicator lights on the garment's display (see above) alert the caregiver to stop the lift procedure.

The wearable lift device also has textile electrodes which are incorporated in the wearable lift device to monitor ECG signals. Textile electrodes composed of stainless steel yarns and a nonconducting fiber are located on the webbing of the wearable lift device over the chest and abdomen of the patient. Microcircuitry in the wearable lift device transmits ECG signals to the microcontroller where heart rate and ECG morphology are analyzed. Textile sensors of ECG signals are described (see e.g., Coyle et al., Ibid.). ECG data is transmitted to a health porvider network (i.e., hospital, clinic) and to the patient's caregiver. If ECG parameters fall outside of a safe, physiological range the microcontroller indicates an unsafe condition on the lift garment display. For example, a heart rate below 40 beats per minute is indicated by red LEDs in the fabric display. Red lights indicate the lift procedure is to be stopped or reversed. Moreover, the microcontroller signals the lift apparatus to lock the lift, and subsequently slowly reverse the lift process. Sensors comprised of piezoelectric fibers are also incorporated in the wearable lift device opposite the chest and abdomen of the patient. Knitted piezoresistive fabric sensors to monitor respiration are described (see e.g., Pacelli, et al., Ibid.). Respiration data is processed and transmitted from the microcontroller to the health provider network and the patient's caregiver. Rapid breathing or abnormal breathing is recognized by the microcontroller and indicated on the garment display. Additionally the microcontroller can lock the lift or reverse the direction of movement based on abnormal respiration data.

Physiological data collected before, during and after the lift procedure is transmitted to the health provider network and the patient's caregiver. The associated records (e.g., electronic files) document the patient's wellbeing and any accidents or injuries that may occur. More importantly, the wearable lift device monitors physiological data and responds instantaneously to an unsafe condition by locking and/or reversing the lift apparatus.

All of the above U.S. patents, U.S. patent application publications, U.S. patent applications, foreign patents, foreign patent applications and non-patent publications referred to in this specification and/or listed in any Application Data Sheet, are incorporated herein by reference, to the extent not inconsistent herewith.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A lift garment, comprising:
a fabric-like material shaped to substantially completely encircle a torso and at least a portion of arms and legs of a subject, the fabric-like material including one or more lift attachment sites;
at least one lift attachment element associated with the fabric-like material at at least one of the one or more lift attachment sites, the at least one lift attachment element configured to attach the lift garment to a lift apparatus;
one or more physiological sensors configured to measure at least one physiological parameter of the subject;
a microcontroller including circuitry configured to receive and process information regarding the measured at least one physiological parameter of the subject; and
a reporting device operably coupled to the microcontroller and configured to transmit one or more signals indicative of the processed information regarding the measured at least one physiological parameter of the subject.

2. The lift garment of claim 1, wherein the microcontroller includes
a stored range of acceptable physiological parameter values; and
circuitry configured to receive and process the information regarding the measured at least one physiological parameter of the subject and
circuitry configured to compare the measured at least one physiological parameter of the subject and the stored range of acceptable physiological parameter values; and
wherein the reporting device is configured transmit a control signal to the lift apparatus to control operation of the lift apparatus in response to the comparison of the measured at least one physiological parameter of the subject and the stored range of acceptable physiological parameter values.

3. (canceled)

4. The lift garment of claim 1, wherein the reporting device is configured to transmit at least one of a locking signal, an unlocking signal, an on/off signal, an up/down signal, an acceleration signal, or a speed signal to the lift apparatus.

5.-10. (canceled)

11. The lift garment of claim 1, wherein the one or more lift attachment sites are associated with a front portion of the fabric-like material.

12. The lift garment of claim 1, wherein the at least one lift attachment element includes at least one of a hook, a loop of material, or a magnet.

13.-14. (canceled)

15. The lift garment of claim 1, wherein the one or more physiological sensors include at least one of a heart rate sensor, a blood pressure sensor, a temperature sensor, a respiration sensor, or a biochemical sensor.

16.-20. (canceled)

21. The lift garment of claim 1, wherein the one or more physiological sensors include at least one blood oxygenation sensor.

22. The lift garment of claim 21, wherein the at least one blood oxygenation sensor is associated with at least one load path between the one or more lift attachment sites.

23. The lift garment of claim 21, wherein the at least one blood oxygenation sensor includes a near infrared optical blood oxygenation sensor.

24. The lift garment of claim 21, wherein the at least one blood oxygenation sensor is associated with a surface of the fabric-like material configured for placement in contact with an external surface of the subject.

25. (canceled)

26. The lift garment of claim 1, wherein the reporting device includes one or more color-coded lights.

27. The lift garment of claim 1, wherein the reporting device includes an audio reporting device including at least one speaker.

28. (canceled)

29. The lift garment of claim 1, wherein the reporting device includes a display.

30. The lift garment of claim 1, wherein the reporting device includes a transmission unit including an antenna.

31. The lift garment of claim 1, wherein the reporting device is configured to communicate with an external device.

32. (canceled)

33. The lift garment of claim 31, wherein the reporting device is configured to communicate with the lift apparatus.

34. The lift garment of claim 31, wherein the reporting device is configured to communicate with a mobile communication device.

35.-36. (canceled)

37. The lift garment of claim 1, wherein the reporting device is configured to communicate with a health provider network.

38.-41. (canceled)

42. The lift garment of claim 1, further including a reinforcing material attached to or incorporated into at least one load path between the one or more lift attachment sites, wherein the reinforcing material extends along at least a portion of the at least one load path.

43.-46. (canceled)

47. The lift garment of claim 1, further including a structural platform is attached to a surface of the fabric-like material.

48. The lift garment of claim 1, further including a structural platform spanning a space between two or more edges of the fabric-like material.

49. The lift garment of claim 1, further including a structural platform including one or more of the one or more physiological sensors, the microcontroller with the circuitry, or the reporting device.

50. (canceled)

51. The lift garment of claim 1, further including at least one load sensor configured to measure a load, the at least one load sensor associated with at least one of the one or more lift attachment sites or at least one load path between the one or more lift attachment sites;
wherein the microcontroller includes circuitry configured to receive and process information regarding the measured load; and
wherein the reporting device is configured to transmit one or more signals indicative of the processed information regarding the measured load.

52.-57. (canceled)

58. A method implemented with a lift garment, comprising:
measuring at least one physiological parameter of a subject with one or more physiological sensors incorporated into the lift garment worn by a subject, the lift garment including
a fabric-like material shaped to substantially completely encircle a torso and at least a portion of arms and legs of the subject;
the one or more physiological sensors;
at least one lift attachment element associated with the fabric-like material at at least one of one or more lift attachment sites;
a microcontroller including circuitry and a stored range of acceptable physiological parameter values; and
a reporting device operably coupled to the microcontroller;
receiving and processing information regarding the measured at least one physiological parameter of the subject with the circuitry of the microcontroller; and
transmitting one or more control signals from the reporting device to a lift apparatus based on the processed information regarding the measured at least one physiological parameter of the subject.

59. The method of claim 58, further including:
determining whether the measured at least one physiological parameter of the subject falls within the stored range of acceptable physiological parameter values; and
transmitting one or more control signals from the reporting device to the lift apparatus to control operation of the lift apparatus if the measured at least one physiological parameter of the subject fails to fall within the stored range of acceptable physiological parameter values.

60. The method of claim 58, further including:
measuring at least one physiological parameter of the subject predictive of hypoxia with the one or more physiological sensors;
receiving and processing information associated with the measured at least one physiological parameter of the subject predictive of hypoxia; and
transmitting one or more control signals from the reporting device to the lift apparatus to control operation of the lift apparatus if the processed information associated with the measured at least one physiological parameter of the subject predictive of hypoxia indicates that the subject is experiencing hypoxia.

61.-69. (canceled)

70. The method of claim 58, wherein measuring the at least one physiological parameter of the subject with the one or more physiological sensors incorporated into the lift garment includes: measuring at least one of heart rate, blood pressure, temperature, respiration, or biochemistry of the subject with at least one of a heart rate sensor, a blood pressure sensor, a respiration sensor, a temperature sensor, or a biochemical sensor incorporated into the lift garment.

71. The method of claim 58, wherein measuring the at least one physiological parameter of the subject with the one or more physiological sensors incorporated into the lift garment includes:
measuring oxygen saturation of the subject with one or more blood oxygenation sensors incorporated into the lift garment.

72. The method of claim 58, further including:
receiving and processing information regarding measurement of the at least one physiological parameter of the subject at a first time point;
receiving and processing information regarding measurement of the at least one physiological parameter of the subject at a second time point;
determining if a rate of change between the measurement at the first time point and the measurement at the second time point falls within a range of acceptable rate changes; and
transmitting a control signal from the reporting device to the lift apparatus to control operation of the lift apparatus if the rate of change between the measurement at the first time point and the measurement at the second time point fails to fall within the range of acceptable rate changes.

73. (canceled)

74. The method of claim 58, wherein
transmitting the one or more control signals from the reporting device to the lift apparatus includes:
transmitting at least one of a locking signal, an unlocking signal, an on/off signal, an up/down signal, an acceleration signal, or a speed signal from the reporting device to the lift apparatus based on the processed information regarding the measured at least one physiological parameter of the subject.

75.-76. (canceled)

77. The method of claim 58, further including:
measuring a load value with a load sensor associated with the lift garment at at least one of the one or more lift attachment sites or at at least one load path between the one or more lift attachment sites; and
transmitting one or more control signals from the reporting device to the lift apparatus based on the measured load value.

78.-80. (canceled)

81. A system, comprising:
a lift garment including
a fabric-like material shaped to substantially completely encircle a torso and at least a portion of arms and legs of a subject, the fabric-like material including one or more lift attachment sites;
at least one lift attachment element associated with the fabric-like material at at least one of the one or more lift attachment sites, the at least one lift attachment element configured to attach the lift garment to a lift apparatus;
one or more physiological sensors configured to measure at least one physiological parameter of the subject;
a microcontroller including a stored range of acceptable physiological parameter values and circuitry configured to receive and process information regarding the measured at least one physiological parameter of the subject; and
a reporting device operably coupled to the microcontroller and configured to transmit one or more signals indicative of the processed information regarding the measured at least one physiological parameter of the subject; and
a lift control mechanism including
a receiver configured to receive the one or more signals from the reporting device indicative of the processed information regarding the measured at least one physiological parameter of the subject; and
circuitry configured to control a function of the lift apparatus in response to the one or more signals received from the reporting device.

82. The system of claim 81, wherein the microcontroller of the lift garment includes
circuitry configured to receive and process the information regarding the measured at least one physiological parameter of the subject, and
circuitry configured to determine whether the measured at least one physiological parameter of the subject falls within the stored range of acceptable physiological parameter values; and
wherein the reporting device operably coupled to the microcontroller is configured to transmit one or more control signals to the lift control mechanism to change operation of the lift apparatus if the measured at least one physiological parameter fails to fall within the stored range of acceptable physiological parameter values.

83. (canceled)

84. The system of claim 81, wherein the one or more physiological sensors includes at least one blood oxygenation sensor associated with at least one load path between the one or more lift attachment sites, the at least one blood oxygenation sensor configured to measure oxygen saturation levels of the subject;
wherein the microcontroller of the lift garment includes a stored range of acceptable oxygen saturation levels and circuitry configured to receive and process information regarding the measured oxygen saturation level of the subject; and
wherein the reporting device operably coupled to the microcontroller is configured to transmit one or more signals indicative of the processed information regarding the measured oxygen saturation level of the subject.

85. The system of claim 84, wherein the microcontroller of the lift garment includes circuitry configured to determine whether the measured oxygen saturation level of the subject falls within the stored range of acceptable oxygen saturation levels; and wherein the reporting device operably coupled to the microcontroller is configured to transmit one or more control signals to the lift control mechanism to change operation of the lift apparatus if the measured oxygen saturation level of the subject fails to fall within the stored range of acceptable oxygen saturation levels.

86.-87. (canceled)

88. The system of claim 81, wherein the lift control mechanism is associated with the lift garment and configured to wirelessly communicate with the lift apparatus.

89. The system of claim 81, wherein the lift control mechanism is associated with the lift apparatus and configured to wirelessly communicate with the lift garment.

90. The system of claim 81, wherein the reporting device operably coupled to the microcontroller is configured to transmit at least one of an on/off signal, an up/down signal, a speed signal, or an acceleration signal to the lift control mechanism based on the processed information regarding the measured at least one physiological parameter of the subject and wherein the lift control mechanism is configured to control at least one of an on/off function, an up/down function, a speed function, or an acceleration function of the lift apparatus.

91.-105. (canceled)

106. The system of claim 84, wherein the at least one blood oxygenation sensor includes a near infrared optical blood oxygenation sensor.

107.-108. (canceled)

109. The system of claim 81, wherein the reporting device operably coupled to the microcontroller includes a transmission unit including an antenna.

110.-113. (canceled)

114. The system of claim 81, wherein the lift garment includes a load sensor configured to measure a load, the load sensor associated with at least one of the one or more lift attachment sites or at least one load path between the one or more lift attachment sites;
wherein the microcontroller includes circuitry configured to receive and process information regarding the measured load; and
wherein the reporting device is configured to transmit one or more signals indicative of the processed information regarding the measured load.

115.-117. (canceled)

118. The lift garment of claim 1, where the fabric-like material is an article of clothing shaped to encircle the torso and at least a portion of the arms and the legs of a human subject, the article of clothing including
at least one lift attachment element associated with the article of clothing and configured to attach the article of clothing to the lift apparatus;
one or more physiological sensors configured to measure at least one physiological parameter of the human subject;
a microcontroller including circuitry configured to receive and process the information regarding the measured at least one physiological parameter of the human subject; and
a reporting device operably coupled to the microcontroller and configured to transmit one or more control signals to the lift apparatus based on the processed information regarding the measured at least one physiological parameter of the human subject.

119. The method of claim 58, further comprising:
measuring the oxygen saturation level of the subject with one or more blood oxygenation sensors incorporated into the lift garment worn by the subject, the lift garment including
the fabric-like material shaped to substantially completely encircle the torso and at least a portion of the arms and the legs of the subject;
the one or more blood oxygenation sensors;
the at least one lift attachment element associated with the fabric-like material at the at least one of one or more lift attachment sites;
the microcontroller including circuitry and a stored range of acceptable oxygen saturation levels; and
the reporting device operably coupled to the microcontroller;
receiving and processing information regarding the measured oxygen saturation level of the subject with the circuitry of the microcontroller; and
transmitting one or more control signals from the reporting device to a lift apparatus based on the processed information regarding the measured oxygen saturation level of the subject.
